(19) United States
(12) Patent Application Publication
(10) Pub. No.: US 2016/0334404 A1
(43) Pub. Date: Nov. 17, 2016

(54) VALUE-ASSIGNED SOLUTIONS OF LIPOPROTEIN-ASSOCIATED PHOSPHOLIPASE A2 HAVING A LONG SHELF-LIFE

(71) Applicants: Thomas D. SCHAAL, San Francisco, CA (US); Shaoqiu ZHUO, Moraga, CA (US)

(72) Inventors: Thomas D. SCHAAL, San Francisco, CA (US); Shaoqiu ZHUO, Moraga, CA (US)

(21) Appl. No.: 15/225,418

(22) Filed: Aug. 1, 2016

Related U.S. Application Data
(63) Continuation of application No. 14/279,106, filed on May 15, 2014, now abandoned.
(60) Provisional application No. 61/881,881, filed on Sep. 24, 2013.

(51) Int. Cl. G01N 33/573; C12N 9/18

(52) U.S. Cl. CPC G01N 33/573; C12Y 301/01004; C12N 9/18; G01N 2333/918

(57) ABSTRACT

Value-assigned solutions having predetermined concentrations of recombinant Lp-PLA2 are described herein. In particular, described herein are solutions of rLp-PLA2 that are stable for an extended period of time. Kits and assays include these calibration solutions, as well as methods of making and using them are described.

### CROSS REFERENCE TO RELATED APPLICATIONS

This patent application is a continuation of U.S. patent application Ser. No. 14/279,106, filed May 15, 2014, and titled “VALUE-ASSIGNED SOLUTIONS OF LIPOPROTEIN-ASSOCIATED PHOSPHOLIPASE A2 HAVING A LONG SHELF-LIFE”, Publication No. US-2015-0086998-A1, which claims priority U.S. Provisional Patent Application No. 61/881,881, filed on Sep. 24, 2013, and titled “CALIBRATION STANDARDS FOR THE DETECTION OF LIPOPROTEIN-ASSOCIATED PHOSPHOLIPASE A2”. This application is herein incorporated by reference in its entirety.

### SEQUENCE LISTING

The instant application contains a Sequence Listing which has been submitted electronically in ASCII format and is hereby incorporated by reference in its entirety. Said ASCII copy, created on Jun. 13, 2014, is named 12248-702.200_SL.txt and is 12,023 bytes in size.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are compositions, kits, assays and methods of making and using them including calibration solutions having a long shelf-life that are stably maintain a predetermined level of functional, properly-folded lipoprotein-associated phospholipase A2 (Lp-PLA₂) over an extended period of time, and specifically the use of such calibration solutions as calibration standards in assays, e.g. ELISA (mass) assays, activity assays, or the like, for detection of Lp-PLA₂.

### BACKGROUND

Lipoprotein-associated phospholipase A2 (Lp-PLA2 or LP-PLA2) is an enzymatically active 50 kD protein that has been associated with Coronary vascular disease (CVD) including coronary heart disease (CHD) and stroke. Lp-PLA2 has been previously identified and characterized in the literature by Tew et al. (1996) Arterioscler. Thromb. Vasc. Biol. 16:591-599, Tjoelker, et al. (1995) Nature 374(6522):549-53), and Caslake et al. (2000) Atherosclerosis 150(2): 413-9. In addition, the protein, assays and methods of use have been described in the patent literature WO 95/00649-A1: U.S. Pat. Nos. 5,981,252, 5,968,818, 6,177,257, 7,052,862, 7,045,329, 7,217,535, 7,416,853; WO 00/24910-A1: U.S. Pat. No. 5,532,152; 5,605,801; 5,641,669; 5,656,431; 5,698,403; 5,977,308; and 5,847,088; WO 04/089184; WO 05/001416: U.S. Pat. No. 7,531,316; WO 05/074604; WO 05/113797; the contents of which are hereby incorporated by reference in their entirety. Lp-PLA2 is expressed by macrophages, with increased expression in atherosclerotic lesions (Hakkinen (1999) Arterioscler Thromb Vase Biol 19(12): 2909-17). Lp-PLA2 circulates in the blood bound mainly to LDL, co-purifies with LDL, and is responsible for >95% of the phospholipase activity associated with LDL (Caslake 2000).

There are a handful of tests, both “mass” (e.g., ELISA-type) assays and activity (e.g., enzymatic activity) assays that have been described. For example, the United States Food and Drug Administration (FDA) has granted clearance for the PLAC® Test (diaDexus, South San Francisco, Calif.) for the quantitative determination of Lp-PLA2 in human plasma or serum, to be used in conjunction with clinical evaluation and patient risk assessment as an aid in predicting risk for coronary heart disease, and ischemic stroke associated with atherosclerosis. Although various assays for detecting Lp-PLA2 protein have been described, such assays typically describe using only freshly isolated or produced (e.g., within a few minutes, hours or days) Lp-PLA2 to form calibration standards.

To provide meaningful results, quantitative Lp-PLA2 assays need to be calibrated and quality controlled with value assigned reagents. Further the assay needs to measure controls within a predetermined quantitative range. The controls and calibrators (standards) can be provided within a reagent kit, as a separate kit or be acquired as individual value assigned reagents. Alternatively Lp-PLA2 reagent kit can be calibrated during manufacturing and calibration values or curves are provided. In this case no physical reagent is used by the laboratory running the assay, rather the kit manufacturer uses in-house reagents to generate calibration curves, values or equivalent for their Lp-PLA2 assay. For example, Lp-PLA2 assays include: immunoassays (Caslake, 2000), activity assays (PAF Acetylhydrolase Assay Kit, Cat #760901 product brochure, Cayman Chemical, Ann Arbor, Mich., 12/18/97; Azwell/Alfresa Auto PAF-AH kit available from the Nesco Company, Alfresa, 2-24-3 Sho, Ibaraki, Osaka, Japan or Karlan Chemicals, Cottonwood, Ariz., see also Kosaka (2000)), spectrophotometric assays for serum platelet activating factor acetylhydrolase activity (Clin Chem Acta 296: 151-161, WO 00/32808 (to Azwell)), and other published methods to detect Lp-PLA2 include WO 00/032808, WO 03/048172, WO 2005/001416, WO 05/074604, WO 05/113797, U.S. Pat. Nos. 5,981,252 and 5,880,273 and U.S. publication No. US 2012-0276569 A1.

As described in greater detail herein, one significant problem, previously not well characterized, with such assays is that the calibrators, standards and controls have a relatively short “shelf-life” once made, as the Lp-PLA2 within even buffered calibration standards loses activity and antigenicity after a few months (e.g., beyond 4-6 months) to a substantial degree. This effect may be particularly true when using recombinant Lp-PLA2. This loss of activity may result in less accurate or even erroneous results when attempting to calibrate or quality control an Lp-PLA2 assay. Thus, it would be beneficial to provide calibration standards and controls, kits including calibration standards and controls, assays including calibration standards and controls, and methods of making and using them, that include the use of recombinant Lp-PLA2 that have long shelf-life and retain stability and activity for more than 4 months (e.g., for more than 5 months, more than 6 months, more than 12 months, more than 18 months, etc.).

### SUMMARY OF THE DISCLOSURE

Calibration solutions, standards, or assay controls having predetermined concentrations of recombinant Lp-PLA2 are described herein, as well as methods and kits using them, including methods of calibrating, re-calibrating or confirming results. In particular, described herein are calibration and control solutions of recombinant Lp-PLA2 (rLp-PLA2) that are stable for an extended period of time (e.g., greater than 4 months, 5 months, 6 months, 7 months, 8 months, 9 months, 10 months, 11 months, 12 months, 18 months, etc.). The terms calibration standards, calibrator and standard may be used interchangeably in this document. In general, the solutions described herein are adapted so that the recombinant Lp-PLA2 may have an exceptionally long shelf-life compared to previously described solutions, and these solution may be directly used in an assay for determining, calibrating or confirming Lp-PLA2 activity and/or concentration. Surprisingly, as described and illustrated below, stability of rLp-PLA2 may be enhanced (even in low-salt solutions) by including a sufficiently high concentration of a detergent, and particularly a cholate detergent, that it forms micelles. This is surprising in part because common wisdom when describing storage and use of protein samples and standards as part of an assay (such as an ELISA-type assay) is to use detergent at relatively low concentrations (and certainly below the critical micelle concentration) so as to avoid potentially deleterious effects of the detergent on the binding. See, e.g., “inhibition of Protein-Protein Interactions: Non-Cellular Assay Formats” in the Assay Guidance Manual, Arkin, et al. (2012) (“Low concentrations of detergents tend to stabilize proteins, reduce nonspecific binding of proteins to assay plates, and break up compound aggregates . . . in general, detergents should be used at concentrations below their critical micelle concentration (CMC).”). The dilution of concentrated recombinant enzyme, known as lipoprotein-associated phospholipase-A2 (Lp-PLA₂), is an integral step in the process of creating calibration standards for an in vitro diagnostic assay used to detect the analyte in a clinical setting. Accurate and stable calibration standards are essential for the requisite traceability necessary when performing clinical in vitro diagnostic assays for the analyte, such as a PLAC (Lp-PLA2 immunoassay) test. Specific individual detergents, formulated at concentrations at, or above, their adjusted critical micelle concentration are required for stabilization and maintenance of accurate and stable analyte values for the analyte, recombinant Lp-PLA₂ protein, in these buffered calibration standards. Proper and reliable functionality of the calibration standards allow for the accurate detection of clinical analyte values for Lp-PLA₂ in bodily fluids, such as blood samples, plasma samples or serum samples. Here, the utility of using specific detergents at appropriate concentrations at or above their individual salt-adjusted critical micelle concentrations (CMC's) to stabilize recombinant Lp-PLA₂ in the context of a calibration standard is demonstrated. Upon dilution, these detergents must be utilized at or above critical micelle concentration in order to protect the enzyme from inactivation, suggesting that detergent micelles stabilizing Lp-PLA₂ protein, not individual detergent molecules (monomers). After the enzyme has been diluted and allowed to become inactivated, the subsequent addition of detergents cannot recover the enzymatic activity of recombinant Lp-PLA₂.

The dilution of recombinant Lp-PLA₂ enzyme in the absence of certain detergents results in inactivation of the enzyme via a bifurcated pathway. Mechanistically, the first route for loss of enzymatic activity in the absence (or, alternatively, at sub-CMC concentrations) of detergent is simply an irreversible denaturation of the recombinant protein due to unfolding. The second route for the loss of enzymatic activity in the absence (or, alternatively, at sub-CMC concentrations) of detergent is an irreversible self-association of Lp-PLA₂ by the formation of dimers and/or higher order oligomers. Importantly, the monomeric Lp-PLA₂ protein has a propensity to dimers and higher order oligomers in the absence of certain detergents, certain polar lipids and/or certain lipoproteins (e.g., binding to HDL or LDL, per its physiological context). The presence of specific detergents at or above their salt-adjusted CMC value is essential for preventing both of these irreversible routes of enzyme inactivation. In particular, the structurally-related members of the cholate family of detergents, including CHAPS, CHAPSO and sodium (deoxy)cholate have been demonstrated to provide excellent stability in preventing the inactivation of the Lp-PLA₂ protein by denaturation and/or formation of higher order oligomers.

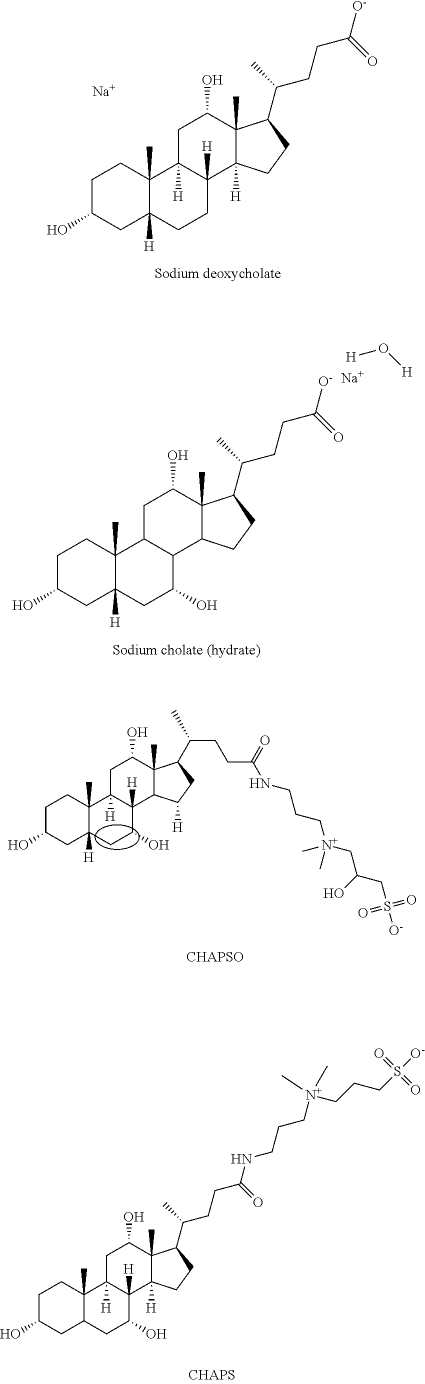

Association of Lp-PLA₂ with Detergent Micelles.

Size exclusion chromatography was performed to estimate the molecular size of the rLp-PLA₂ the presence and absence of 10 mM CHAPS (CMC=˜6 mM). The results indicated that the same enzyme was eluted very differently under the various conditions. The expected molecular weight of Lp-PLA₂, not including the glycosylation oligosaccharide chains, is about 48 kD. To further understand the retention time shift, we resolved the enzyme by the same procedure with different detergents. The results showed that the column retained rLp-PLA₂ differently with different detergents. Detergents with larger micelle molecular weight eluted rLp-PLA₂ earlier from the column. This indicates the association of rLp-PLA₂ with the micelles of the detergents. However, the molecular size of the rLp-PLA₂ in the absence of the detergents seems even larger than that of the complex containing the enzyme:detergent micelle. This suggests that the enzyme forms oligomeric structures or aggregates in the absence of detergents. In addition, the recovery yield based on the enzymatic activity assay was much lower when rLp-PLA₂ was fractionated in the absence of detergents. In the absence of detergents, only about 23% of rLp-PLA₂ activities were recovered compared to 60-146% recovery in the presence of detergents. Thus, dilution of rLp-PLA₂ in the absence of detergents results in irreversible inactivation of the enzyme.

To investigate the lost rLp-PLA₂ in the absence of detergents, purified rLp-PLA₂ with a His-tag at the C-terminal was subjected to fractionation and the fractions were assayed by both the CAM assay and the His-ELISA using rabbit anti-Lp-PLA₂ polyclonal antibody. When rLp-PLA₂ was fractionated in the absence of detergents, the results indicated that two mass peaks (fraction 16-18 and 21-23) were shown by the His-ELISA but only one activity peak (fraction 16-18) was seen by the CAM assay. That is, the lower molecular weight mass peak (fraction 21-23) contained no enzymatic activity. However, when the enzyme was fractionated in the presence of 10 mM CHAPS in the same buffer, no mass or enzymatic activity at fraction 16-18 was seen but both mass and enzymatic activity were detected at the fraction 21-23. This suggests that the lower molecular weight peak (fraction 21-23), which probably comes from the higher molecular weight peak (fraction 16-18), losses its activity irreversibly in the absence of detergents. In the presence of detergents, rLp-PLA₂ is probably does not form oligomers, and, furthermore, it is stabilized by the formation of the complexes with detergent micelles.

Dilution Results in Inactivation of rLp-PLA₂ in the Absence of Detergents.

Freshly prepared rLp-PLA₂ diluted in the presence or absence of detergents had no difference in specific activity when assayed with CAM (results not shown). However, when the enzyme is stored in the absence of detergents at 4° C. it lost its activity faster, especially at low analyte concentrations (results not shown). To further investigate the decrease of rLp-PLA₂ specific activity in the absence of detergents, the enzyme was subjected to dilution to the final concentration between 1-3 μg/ml in PBS, pH 7.2, and the changes of the enzymatic activity and immuno-reactive mass were followed. The immuno-reactive mass of Lp-PLA₂ was quantified by using the PLAC kits that only recognized the non-denatured form of the enzyme (conformational). The enzyme gradually lost its activity and immuno-reactive mass in two phases. Upon dilution, the enzymatic activity and the immuno-reactive mass had a sharp decline phase (about 1-2 days of incubation at 4° C.) and then the inactivation rate decreased and transferred to a slower phase. The final normalized losses in both activity and immuno-reactive mass were in the range of 50-75% at the fifteenth day of incubation. Actually, for each reaction, the inactivation rates and final losses of the enzymatic activity and immuno-reactive mass varied with different experimental conditions depending on the final diluted enzyme concentration (see the following experiments), the storage conditions of the enzyme, the dilution buffer components and incubation temperature, etc.

Detergents have Differential Effects on rLp-PLA₂ Activity.

The effects of detergents on the dilution inactivation of rLp-PLA₂ were investigated. When 10 mM CHAPS was included in the dilution buffer, no inactivation was observed for the diluted rLp-PLA₂ at 1 μg/ml. However, the addition of 10 mM CHAPS into the inactivated enzymes only recovered a very small portion of the lost activity but it did prevent the enzyme from further inactivation during the extended incubation. In addition to CHAPS, several other non-ionic detergents, such as Tween-20, Triton X-100 and digitonin, were also found protective in the dilution inactivation of rLp-PLA₂ (data not shown). Detergents with high CMC were less effective than those with lower CMC. In an experiment of dilution inactivation for rLp-PLA₂, the diluted enzyme was incubated in buffers containing variable detergent concentrations from 0.15 mM to 10 mM. The rate of enzyme inactivation was found to be concentration dependent for CHAPS (CMC=6 mM) and deoxycholate (CMC=1.5 mM) but not for Triton X-100 (CMC=0.3 mM), Digitonin (CMC=0.09 mM) and Tween-20 (CMC=0.06 mM). This suggests that detergent micelles, possibly instead of or in addition to monomeric detergent, are the stabilizer of rLp-PLA₂ molecule.

The Effects of the Protein Concentration on the Activity of rLp-PLA₂.

At high concentrations (>0.5 mg/ml), rLp-PLA₂ is fairly stable even in the absence of detergents (observation not shown). In the dilution inactivation of the recombinant Lp-PLA₂, the inactivation rates are dependent on the final diluted concentration of the enzyme. The concentration effect on the rLp-PLA₂ dilution inactivation is illustrated in. The rate and final loss of the rLp-PLA₂ inactivation upon dilution varied in the enzyme concentration range of 0.6-5 μg/ml. The inactivation rates became relatively independent of final enzyme concentrations at both ends of the above concentration range. This can be better demonstrated by plotting the residual residue percentage of the rLp-PLA₂ activity after the enzyme was diluted and incubated at 4° C. for ten days against the protein concentrations. In the logistic scale of concentration, it can be fitted into a sigmoidal curve. There is a sensitive range between 1 and 5 The saturation at both concentration ends may indicate that there is a dynamic equilibrium between the stable and unstable forms of rLp-PLA₂, which shifts depending on the concentration of the enzyme. Since the inactivation is due to structural disruption by solvent and irreversible, it should be a reaction of first order kinetics, that is, concentration independent. When the enzyme concentration decreases to a certain level, the equilibrium is shifted to the unstable form and then the irreversible inactivation rate becomes concentration independent. When the concentration of rLp-PLA₂ increases, the rate of inactivation is reduced due to the equilibrium shifting to the stable form of the enzyme. Most likely, the stable and unstable forms of Lp-PLA₂ should represent the oligomerized and the dissociated enzyme respectively since the dilution usually causes dissociation and vice versa. At the concentration at 2.5 μg/ml or 53 nM, roughly half of rLp-PLA₂ is in monomer and half forms the aggregate or oligomer as estimated.

Protection of rLp-PLA₂ Activity by Lipoproteins.

Lp-PLA₂ protein has been shown to associate with LDL and HDL in human plasma (9). Experiments were designed to reveal if LDL and HDL would prevent rLp-PLA₂ from the inactivation during the dilution into non-detergent containing buffers. Purified rLp-PLA₂ was diluted in 50 mM sodium phosphate buffer, pH 7.2, containing 150 mM sodium chloride and 2 mM EDTA at the final concentration of 0.5 μg/ml enzyme and incubated at 4° C. for 2 days. The experiments were carried out in the presence of various concentrations of fractionated LDL and HDL (devoid of endogenous Lp-PLA₂ activity). It was indeed found that the dilution inactivation of rLp-PLA₂ could be averted in the presence of either LDL or HDL particles. Human LDL or HDL at concentrations as low as 1.4 and 0.14 mg/dL of triglyceride respectively fully protected the rLp-PLA₂ activity during the dilution in the phosphate buffer. No significant activity losses were observed after the two day period of incubation at 4° C. in the LDL or HDL containing buffer while more than 90% of the original activity vanished in the control buffer. However, unexpectedly higher concentrations of LDL or HDL reduced the protection capability possibly due to the proteolysis of the recombinant enzyme.

The Effects of Chaotropic Agents on the Activity of rLp-PLA₂.

According to the gel permeation experiments, detergents could reduce the molecular weight of rLp-PLA₂ and stabilize its activity. To investigate the connection between the deoligomerization and stabilization effects of detergents, rLp-PLA₂ was diluted and incubated at 4° C. in the presence of 1 M sodium salts of fluoride, bromide, chloride, iodide, nitrate, sulfate (0.5 M) and thiocyanate. While detergents were found to stabilize rLp-PLA₂, anions destabilizing protein-protein interactions, such as SCN⁻¹ or I⁻¹ (22), were found to promote the inactivation of the enzyme. The inactivation of the diluted rLp-PLA₂ during the incubation at 4° C. was significantly accelerated by including 1 M of NaSCN or Nal in the incubation buffer. This is not due to the added sodium salt concentration because no other salts had effects on the stability of the enzyme. None of the above chemicals (up to 1 M) was found inhibitory to the enzymatic activity of rLp-PLA₂ either (results not shown). The experiment suggests that the protein-protein interaction breaker such as SCN⁻¹ or I⁻¹ actually destabilizes rLp-PLA₂. It may be inferred that rLp-PLA₂ tenders to form a dimer or oligomers during the incubation but, if the self-interaction is prevented or interrupted by chaotropic agents, the monomeric enzyme will be denatured, possibly due to exposure of the hydrophobic substrate binding site to aqueous solvents.

Chemical Cross-Linking of rLp-PLA₂.

To further confirm the formation of the oligomeric rLp-PLA₂ during dilution, the highly purified enzyme was diluted into buffers containing a chemical cross-linker, ethylene glycol bis[succinimidylsuccinate] (EGS), with and without detergents. In a cross-linking experiment, when rLp-PLA₂ was diluted to the final concentration of 1 μg/ml in the absence of detergents, only oligomers with molecular weight >98 kD were detected on the Western Blot by rabbit anti-Lp-PLA₂ antibody. No monomeric (48 kD) and only a low amount of dimeric (98 kD) rLp-PLA₂ were seen. Second, the extent of rLp-PLA₂ oligomerization observed was different when stored at different conditions. Enzyme stored in buffer containing 5 mM CHAPS had a lower oligomerized molecular weight than enzyme stored in the detergent-free condition although both were diluted into the same cross-linking buffer at the same final concentration. Third, in the presence of 10 mM CHAPS (or 1% Tween-20, data not shown), the majority of rLp-PLA₂ stayed monomeric after cross-linked by EGS. Again, the enzyme stored in the presence of 5 mM CHAPS was almost free of oligomeric bands when cross-linked in buffer containing detergents while the detergent-free enzyme still had significant amounts of high molecular weight species when cross-linked in the same buffer. These results prove that rLp-PLA₂ does quickly self-associate and form polymers upon dilution in the absence of lipid substrates or detergents. The detergents do not reduce the reactivity of EGS in the cross-linking of rLp-PLA₂ because the control experiments to internally cross-link IgG by EGS were not altered by the presence of the same detergents (data not shown). Thus, the purified recombinant lipoprotein-associated phospholipase A2 (rLp-PLA₂) expressed in HEK293 cells has a propensity to form oligomers in the absence of detergents or lipids by chemical cross-linking.

A Detergent Comparison study was a component swapping experiment in which selected membrane detergents/CHAPS analogues were screened in a short-term real-time stability study. The eleven different detergent variants in this study included each of those included in the Dojindo “First Choice” detergent screening kit (CHAPS, n-Dodecyl-β-D-maltoside, n-Octyl-β-D-glucoside, sodium cholate and MEGA-8), various CHAPS analogues (Dojindo detergents CHAPSO, BIGCHAP, deoxy-BIGCHAP), and various grades/lot numbers of Sigma CHAPS (including two lots from the current grade of CHAPS used in Manufacturing). All the detergents were substituted into the standard calibrator diluent formulation at four concentrations each in a linear titration series. The concentration range surveyed for each detergent was based on each individual detergent's published critical micelle concentration (CMC). Most detergents were also tested at one concentration above the CMC and two concentrations below the CMC, with the single exception being the MEGA-8 detergent. The MEGA-8 detergent presented a technical challenge with respect to testing above its published CMC (58 mM). A key aspect of this study is the detergent concentrations chosen were normalized based on their respective critical micelle concentrations (CMC's), a value specific to each detergent. With respect to maintaining Lp-PLA₂ stability, the general trend for the set of detergents was stabilized was maximal when the detergent concentration was at CMC (or higher) with a sharp drop off in stability at concentrations lower than CMC. This result strongly suggests that micelle formation is important for maintaining Lp-PLA₂ stability across the entire panel of detergents surveyed. When CHAPS was studied to the exclusion of the other detergents, the lots of CHAPS analyzed here actually showed slightly better stability at sub-CMC concentrations than the other detergents. The 0.595× concentration of CHAPS (corresponding to the standard [4.76 mM] CHAPS concentration in the calibrator matrix) showed comparable stability to the 1×CMC concentration, but the stability profile showed ˜10% drop-off at the 0.354× concentration (corresponding to the 2.83 mM CHAPS concentration). These results from this thirty-day stability study suggested that a concentration of CHAPS used in a calibrator diluent formulation (e.g., 4.76 mM) may be close to a “cliff” in CHAPS concentration with respect to stability performance.

The Detergent Comparison study also demonstrated that there is differential calibrator stability observed when using different lots of CHAPS detergent. In a comparison of four different lots of CHAPS from two vendors, statistically significant differences in stability were obtained using a Student's t-test even within the timeframe a 30-day short-term stability study. Notably, the difference in stability between the Dojindo lot of CHAPS (lot number CT717) and the Sigma lot #3 (BioXtra, lot number 18K530041V) yielded a statistically significant difference at every CHAPS concentration tested. Given that standard concentrations of the other raw materials were used in this study, these results suggest the possibility that differences in stability as a function of detergent concentration can be observed even in a relatively short timeframe. It should be noted, though, that the differential in percent stability observed with some of these lots of Sigma CHAPS (namely, lots 018K53003 and 040M5319V) is of a greater magnitude than that observed in subsequent stability studies with the same two lots of Sigma CHAPS in the Mix-and-Match Study. On the other hand, the single lot of Dojindo CHAPS tested demonstrated good stability at the standard CHAPS concentration and higher when tested using the same pre-formulated master-mixes of the remaining raw materials common to each formulation.

A variety of other detergents were screened in the Detergent Comparison study to assess the feasibility of using alternate detergents to stabilize Lp-PLA₂. The two CHAPS analogues, CHAPSO and sodium cholate, showed promising short-term stability results. The n-octyl-b-glucoside showed some promise with its performance in this initial screen; this detergent was used in the determination of the structure of Lp-PLA₂ by x-ray crystallography (Samanta 2008). The n-octyl-b-maltoside showed less promising short term stability indicated by a sharp drop-off in percent stability between the Day 14 time point and the Day 0 time point. The MEGA-8 may require a relatively high detergent concentration (˜30 mM) for effective protein stabilization.

In general, described herein are calibration solutions having a very long shelf-life. As used herein, shelf-life refers to the time during which the solution stably maintain a predetermined level of functional, properly-folded lipoprotein-associated phospholipase A2 (Lp-PLA₂). Thus the shelf-life may refer to the length of time during which a predetermined amount (e.g., more than 95%, more than 90%, more than 85%, more than 80%, etc.) of the concentration and/or activity of the Lp-PLA2 within the solution is retained.

As described in greater detail below for the first time, a calibration solution of recombinant Lp-PLA2 having a long shelf-life may include a predetermined amount of Lp-PLA2 (e.g., predetermined dilution) in a buffer solution that includes sufficient micelles to stabilize the recombinant Lp-PLA2. The micelles may be made of a cholat detergent (e.g., CHAPS, CHAPSO, sodium (deoxy)cholate, etc.) at a concentration above the critical micelle concentration (CMC), as well as a preservative, salt (e.g., non-chaotropic salt), pH buffer and protein buffered matrix.

For example, described herein are lipoprotein-associated phospholipase A2 (Lp-PLA2) calibrator kits for use with an Lp-PLA2 assay, the kit having a shelf-life of greater than 4 months (e.g., greater than 5 months, greater than 6 months, greater than 7 months, greater than 8 months, greater than 9 months, greater than 10 months, greater than 11 months, greater than 12 months, greater than 13 months, greater than 14 months, greater than 15 months, greater than 16 months, greater than 17 months, etc.). A kit may include: a first calibration solution comprising a first concentration of a recombinant Lp-PLA2 in a first buffer solution, wherein the first buffer solution comprises a plurality of micelles of a first detergent stabilizing the recombinant Lp-PLA2; and a second calibration solution comprising a, second concentration of the recombinant Lp-PLA2 in a second buffer solution, wherein the second buffer solution comprises a plurality of micelles of a second detergent stabilizing the recombinant Lp-PLA2.

Any of calibrator kits (which may be separate from or included as part of an assay for identifying Lp-PLA2), may include a plurality of calibration solutions, where each solution has a predetermined amount of recombinant Lp-PLA2. For example, a kit may include at least three calibration solutions each having a different but known concentration of Lp-PLA2 in a buffer solution, wherein the buffer solution comprises micelles of a detergent; the micelles act to stabilize the recombinant Lp-PLA2.

In general, the primary detergent (forming the micelles in the buffer) may be any appropriate detergent, including (but not limited to) members of the cholate family of detergents. The concentration of primary detergent is generally above the CMC. Although different buffer solutions for the different calibration concentrations may be used, in general the same calibration buffer compositions may be used, with the exception of the differing concentrations of recombinant Lp-PLA2. For example, the first and second primary detergent may comprise CHAPS (e.g., at a concentration that is above the CMC for the amount of salt in the buffer solution).

In any of the variations described herein, the calibration buffer solutions including the recombinant Lp-PLA2 may be “low salt” (e.g., less than 1 M salt concentration) buffer solutions. As described in greater detail below, such low-salt solutions may include a second detergent (e.g., a surfactant such as TWEEN 80) to prevent aggregation of the recombinant Lp-PLA2, in addition to the detergent forming the micelles. Any of the buffer solutions described herein may include a salt that is a non-chaotropic salt. For example, the buffer solution may include comprises one or more of: NaCl and an acetate salt. Any of the calibration solutions described herein may also include a preservative (e.g., sodium azide).

The buffer solutions described herein may also typically include a protein buffered matrix, such as a bovine serum albumin (BSA). Any of the calibration buffer solutions described herein may also include a pH buffer (e.g., Tris).

For example, a lipoprotein-associated phospholipase A2 (Lp-PLA2) calibrator kit for use with an Lp-PLA2 assay, having a shelf-life of greater than 4 months, may include: a first calibration solution comprising a first concentration of a recombinant Lp-PLA2 in a first buffer solution, wherein the first buffer solution comprises a plurality of micelles of a cholate detergent stabilizing the recombinant Lp-PLA2, a protein buffered matrix, a pH buffer and a preservative; and a second calibration solution comprising a second concentration of the recombinant Lp-PLA2 in a second buffer solution, wherein the second buffer solution comprises a plurality of micelles of the cholate detergent stabilizing the recombinant Lp-PLA2.

Also described herein are lipoprotein-associated phospholipase A2 (Lp-PLA2) assays having recombinant calibrators, the assay comprising: a plurality of calibrator solutions each comprising a predetermined concentration of a recombinant Lp-PLA2 in a buffer solution, wherein the buffer solution comprises a plurality of micelles of a cholate detergent stabilizing the recombinant Lp-PLA2; a wash buffer; a solid phase support configured to bind Lp-PLA2; and a report antibody specific to Lp-PLA2. The calibrator solutions may include any of the calibrator buffer solutions described herein, including in particular a cholate detergent is above a critical micelle concentration (CMC) for the detergent. Although this example describes an immunoassay kit (e.g., a mass kit) other Lp-PLA2 assays may be based on activity (enzymatic activity) and may include a substrate and detection means (e.g., colorimetric, radioactive, etc.) along with the calibration standards.

A lipoprotein-associated phospholipase A2 (Lp-PLA2) assay having recombinant value-assigned solutions having a shelf-life of more than 4 months, the assay may comprise: a plurality of value-assigned solutions each comprising a predetermined concentration of a recombinant Lp-PLA2 in a low-salt buffer solution having a salt concentration below about 1 M, wherein the buffer solution comprises a cholate detergent forming a plurality of micelles that stabilizes the recombinant Lp-PLA2; a solution comprising an agent that interacts with Lp-PLA2 to produce a detectable signal; and a wash buffer.

Also describe are lipoprotein-associated phospholipase A2 (Lp-PLA2) assay utilizing a value-assigned solution having a long shelf-life for use as a standard, control, calibrator or re-calibrator, may include: a value-assigned solution comprising a predetermined concentration of a recombinant Lp-PLA2 in a buffer solution, wherein the buffer solution comprises a cholate detergent forming a plurality of micelles that stabilize the recombinant Lp-PLA2; a wash buffer; a solid phase support configured to bind Lp-PLA2; and a report antibody specific to Lp-PLA2.

As mentioned, low-salt calibration solutions may be used. In general, a low salt calibration solution includes less than 1 M salt in addition to micelles that help stabilize the recombinant Lp-PLA2, and may also include a secondary detergent (e.g., a surfactant such as Tween-20).

For example, a lipoprotein-associated phospholipase A2 (Lp-PLA2) calibrator kit for use with an Lp-PLA2 assay having a shelf-life of greater than 4 months may include: a first calibration solution comprising a first concentration of a recombinant Lp-PLA2 in a first low-salt buffer solution having a salt concentration below about 1 M, wherein the first buffer solution comprises a plurality of micelles of a first detergent stabilizing the recombinant Lp-PLA2 and a first secondary detergent to prevent aggregation of the recombinant Lp-PLA2; and a second calibration solution comprising a second concentration of the recombinant Lp-PLA2 in a second low-salt buffer solution having a salt concentration below about 1 M, wherein the second low-salt buffer solution comprises a plurality of micelles of a second detergent stabilizing the recombinant Lp-PLA2 and a second secondary detergent to prevent aggregation of the recombinant Lp-PLA2.

For example, a lipoprotein-associated phospholipase A2 (Lp-PLA2) calibrator kit for use with an Lp-PLA2 assay, having a shelf-life of greater than 4 months, may include: a first calibration solution comprising a first concentration of a recombinant Lp-PLA2 in a first low-salt buffer solution having a salt concentration below about 1 M, wherein the first buffer solution comprises a plurality of micelles of a cholate detergent stabilizing the recombinant Lp-PLA2, a first secondary detergent to prevent aggregation of the recombinant Lp-PLA2, a protein buffered matrix, a pH buffer and a preservative; and a second calibration solution comprising a second concentration of the recombinant Lp-PLA2 in a second low-salt buffer solution having a salt concentration below about 1 M, wherein the second buffer solution comprises a plurality of micelles of the cholate detergent stabilizing the recombinant Lp-PLA2 and a second secondary detergent to prevent aggregation of the recombinant Lp-PLA2.

Also described herein are methods of calibrating and methods of recalibrating an assay for Lp-PLA2. For example, described herein are methods of recalibrating a calibration curve for detection of lipoprotein-associated phospholipase A2 (Lp-PLA2) from a biological sample using a value-assigned solution of recombinant Lp-PLA2 having a long shelf life. The method for recalibration may include: detecting a first signal from a value-assigned solution having a first predetermined concentration of a recombinant Lp-PLA2 in a buffer solution, wherein the buffer solution comprises a detergent forming a plurality of micelles that stabilize the recombinant Lp-PLA2; and transforming the calibration curve using the first signal.

In general, a method of recalibrating may be used to adjust a predetermined calibration curve. The predetermined calibration curve may be factory or lot defined and may be provided with a kit or collection of reagent used to quantify the level and/or activity of Lp-PLA2. Recalibration generally involves taking one or more measurements (signals) from value-assigned solutions of rLp-PLA2. A value-assigned solution is one in which the amount of rLp-PLA2 is known or set to a predetermined level. Thus, a value-assigned solution may be a standard, a calibration solution, etc.

Any of the recalibration methods described herein may include a step of transforming (e.g., adjusting) a preexisting calibration curve based on the signal(s) collected from one or more value-assigned solutions. Transforming may include shifting, scaling or shifting and scaling the calibration curve based on the first signal. The step of transforming is typically performed by a machine such as a computer (processor) or the like, which may be configured (specifically configured by including software, hardware or firmware) that adjusts an initial calibration curve based on the detected signal(s) from the value-assigned (‘recalibration’) solutions. The known value (e.g., concentration value, activity, etc.) of the value-assigned solution may be correlated with the detected signal. In some variations the calibration curve may be fit (e.g., suing the machine) to the signals measured for the value-assigned solution(s).

Any appropriate calibration curve may be used. For example, a calibration curve may show a relationship between signal (e.g., measured as optical signal, etc.) and concentration of Lp-PLA2 and/or activity of Lp-PLA2. For example, in some variations the calibration curve relates signal intensity to concentration of Lp-PLA2.

Any number of value-assigned solutions may be used to provide calibration/re-calibration signals. For example, the method may include detecting a second (or more) signal from a second value-assigned solution having a second predetermined concentration of a recombinant Lp-PLA2 in the buffer solution, wherein the buffer solution comprises the detergent forming a plurality of micelles that stabilize the recombinant Lp-PLA2, and wherein transforming the calibration curve comprises using the first and second signals.

In general, the methods of calibration and recalibration (as well as methods for normalizing or performing a control) of Lp-PLA2 assays described herein may include combining the value-assigned solution(s) with an agent that interacts with Lp-PLA2 to produce a detectable signal before detecting the first signal. Any appropriate agent may be used, including an agent that interacts with Lp-PLA2 to form a detectable complex, such as an antibody directed against Lp-PLA2 or a substrate for Lp-PLA2. Alternatively, the agent may be or may include a substrate on which the Lp-PLA2 acts. For example, the agent that interacts with Lp-PLA2 may comprise a labeled antibody directed against Lp-PLA2 or a labeled substrate for Lp-PLA2.

Detecting signal may include detecting a complex of Lp-PLA2 and an antibody or detecting enzymatic activity Lp-PLA2.

As described in greater detail herein, value-assigned solutions of rLp-PLA2 that are of particular interest and utility include those having a plurality of micelles of a detergent. Thus, the detergent (a first detergent) may be above the critical micelle concentration (CMC) for the detergent in the context of the value-assigned solution. The detergent may be, in particular a cholate detergent (e.g., CHAPS, etc.). In addition to the micelles of detergent that stabilize the rLp-PLA2, the solutions described herein may also include additional detergent (the same detergent or a different detergent) that prevents aggregation of the Lp-PLA2. Any of the buffer solutions in which the rLp-PLA2 are present may be configured as low-salt solutions (e.g., having less than 1 M total salt). For example, detecting signal from the value-assigned solutions may include detecting the first signal from the value-assigned solution having the first predetermined concentration of a recombinant Lp-PLA2 in the buffer solution, wherein the buffer solution is a low-salt buffer solution having a salt concentration below about 1 M and comprising a detergent forming the plurality of micelles and a second detergent to prevent aggregation of the recombinant Lp-PLA2, further wherein the detergent forming the plurality of micelles is different from the second detergent.

Detecting signal may include detecting the first signal from the value-assigned solution having the first predetermined concentration of a recombinant Lp-PLA2 in the buffer solution, wherein the buffer solution further comprises a protein buffered matrix (e.g., PBS). As described herein, the type and concentration of the protein buffered matrix (or any of the other components of the solution) may be chosen to optimize the shelf-life.

For example, described herein are methods of recalibrating a calibration curve for detection of lipoprotein-associated phospholipase A2 (Lp-PLA2) from a biological sample using a value-assigned solution of recombinant Lp-PLA2 having a long shelf life, the method comprising: combining a value-assigned solution comprising a first predetermined concentration of a recombinant Lp-PLA2 in a buffer solution with an agent that interacts with Lp-PLA2 to produce a detectable first signal, wherein the buffer solution comprises a detergent forming a plurality of micelles that stabilize the recombinant Lp-PLA2; detecting the first signal; and transforming a calibration curve by shifting, scaling or shifting and scaling the calibration curve based on the first signal.

In addition to method of re-calibrating a calibration cure, also described herein are methods of producing calibration curves (for detection of lipoprotein-associated phospholipase A2 (Lp-PLA2) from a biological sample) using the value-assigned solutions of recombinant Lp-PLA2 that have a long shelf life described herein. For example, a method of generating a calibration curve may include: combining an agent that interacts with Lp-PLA2 to produce a detectable signal with a plurality of value-assigned solutions, wherein each value-assigned solution has a predetermined concentration of the recombinant Lp-PLA2 in a buffer solution, the buffer solution comprising a detergent forming a plurality of micelles that stabilize the recombinant Lp-PLA2; detecting Lp-PLA2 signals from the value-assigned solutions; and creating a calibration curved based on the relationship between the detected signals and the predetermined concentrations of the recombinant Lp-PLA2.

In general, detecting Lp-PLA2 signals from the value-assigned solutions may include detecting Lp-PLA2 signals from at least four value-assigned solutions having different predetermined concentrations of the recombinant Lp-PLA2 (e.g., more than four, more than five, more than six, more than seven, more than eight, more than nine, more than ten, etc.). For example, detecting Lp-PLA2 signals may comprise detecting Lp-PLA2 signals from between about four to 10 value-assigned solutions having different predetermined concentrations of the recombinant Lp-PLA2.

Since the methods of detecting Lp-PLA2 activity/amount, method of re-calibrating a calibration curve and methods of generating a calibration curve described herein are specifically for use with the improved solutions (e.g., value-assigned solutions having a long shelf-life) described, the outcome of such methods may be significantly different from other methods that do not use these solutions. For example, although prior art methods for detecting concentration and/or activity of Lp-PLA2 are performed in a low-detergent buffer (e.g., below the CMC), surprisingly the inventors have herein found that assaying activity and/or binding of Lp-PLA2 in the presence of micelles, as well as storing rLp-PLA2 in the presence of micelles, has a beneficial effect. Thus, any of the detection steps for detecting activity and/or binding of Lp-PLA2 may be performed in the presence of micelles of detergent, and/or in a low-salt buffer.

In addition any of the methods described herein may be performed with a value-assigned solution that has been stored for more than four months (e.g., more than five months, more than six months, etc.).

As mentioned, a calibration curve may relate a signal intensity of the signals to the predetermined concentrations of the recombination Lp-PLA2.

In any of the methods described herein, signal may be detected by combining the rLp-PLA2 (or for determining an unknown, a sample from a patient including Lp-PLA2) with an agent generates a detectable signal. The signal may be directly or indirectly detected. For example, the agent may be an antibody that binds or complexes with the rLp-PLA2 (or Lp-PLA2) such as an antibody directed against Lp-PLA2 or a substrate for Lp-PLA2. The agent that interacts with Lp-PLA2 may be, for example, a labeled antibody directed against Lp-PLA2 or a labeled substrate for Lp-PLA2. The label may be optically detectable (e.g., florescent, HRP, etc.) or it may be radio detectable, or the like. In some variations the signal is indirectly detectable as, for example, when the enzymatic activity of the rLp-PLA2 (or endogenous Lp-PLA2) is detectable by detecting a product resulting from the enzymatic activity of the Lp-PLA2.

Detecting LpPLA2 signals may comprise detecting a complex of Lp-PLA2 and an antibody or detecting enzymatic activity Lp-PLA2 on a substrate after combining the solution including rLp-PLA2 with an agent to generate a detectable signal. Combining may comprise combining the agent with each of the plurality of value-assigned solutions, wherein the buffer solution of the value-assigned solutions comprises a plurality of micelles of CHAPS that stabilize the recombinant Lp-PLA2.

The step of combining may comprise combining the agent with each of the plurality of value-assigned solutions, wherein the buffer solution of the value-assigned solutions comprises a low-salt buffer solution having a salt concentration below about 1 M and a second detergent to prevent aggregation of the recombinant Lp-PLA2, further wherein the detergent forming the plurality of micelles that stabilize the recombinant Lp-PLA2 is different from the second detergent.

In some variations, the step of combining may comprise combining the agent with each of the plurality of value-assigned solutions, wherein the buffer solution of the value-assigned solutions comprises a protein buffered matrix.

In general, creating a calibration curve may include arranging the signals from the value-assigned solutions versus the predetermined concentrations of the recombinant Lp-PLA2 in the value-assigned solutions. A curve may be fit to the resulting arrangement. The curve may be first order, second order, third order, etc. A mathematical expression for the curve may be provided (e.g., by the apparatus, e.g., software, firmware, hardware), and this mathematical expression may be used to determine an estimate of the value of Lp-PLA2 concentration and/or activity from a biological sample.

For example, a method of producing a calibration curve for detection of lipoprotein-associated phospholipase A2 (Lp-PLA2) from a biological sample by using value-assigned solutions of recombinant Lp-PLA2 that have a long shelf life may include: combining an agent that interacts with Lp-PLA2 to produce a detectable signal with a plurality of value-assigned solutions, wherein each value-assigned solution has a different predetermined concentration of the recombinant Lp-PLA2 in a buffer solution, the buffer solution comprising a detergent forming a plurality of micelles that stabilize the recombinant Lp-PLA2, a pH buffer, a protein buffered matrix and a non-chaotropic salt; detecting Lp-PLA2 signals from the value-assigned solutions; and creating a calibration curved based on the relationship between the detected signals and the predetermined concentrations of the recombinant Lp-PLA2.

As mentioned above, kits are also described herein. Any of the solutions (including value-assigned solutions of rLp-PLA2) may be included as part of a kit or set. In general a kit may be pre-assembled so that a user is provided with all of the component parts (e.g., in a single container, or connected container) or it may be assembled by the user from different or separately provided components. In general, the kit includes multiple different items that may be used as described herein.

For example, a lipoprotein-associated phospholipase A2 (Lp-PLA2) kit for use with an Lp-PLA2 assay, the kit having a shelf-life of greater than 4 months, may include: a first value-assigned solution comprising a first predetermined concentration of a recombinant Lp-PLA2 in a first buffer solution, wherein the first buffer solution comprises a first detergent forming a plurality of micelles that stabilize the recombinant Lp-PLA2; and a second value-assigned solution comprising a second predetermined concentration of the recombinant Lp-PLA2 in a second buffer solution, wherein the second buffer solution comprises a second detergent forming plurality of micelles that stabilize the recombinant Lp-PLA2. The kit may also include a third (or fourth, fifth, sixth, etc.) value-assigned solution comprising a third predetermined concentration of the recombinant Lp-PLA2 in a third buffer solution, wherein the third buffer solution comprises a third detergent forming a plurality of micelles that stabilize the recombinant Lp-PLA2. The first and second detergents (e.g., the detergents forming the micelles) may comprise a cholate detergent, such as CHAPS, at greater than the CMC for the buffer. In general, the first and second buffer solutions may be the same solution. In particular, the first buffer solution and the second buffer solution may be a low-salt buffer solution (e.g., having a total salt concentration of less than 1 M). The first buffer solution and the second buffer solution comprises a low-salt buffer solution comprising a non-chaotropic salt. The first buffer solution and the second buffer solution may comprise a low-salt buffer solution comprising one or more of: NaCl and an acetate salt. The first buffer solution and the second buffer solution may comprise a protein buffered matrix, e.g., bovine serum albumin (BSA). The first buffer solution and the second buffer solution may include Tris as a pH buffer.

In some variations the kit includes a ‘blank’ that includes the buffer without any rLp-PLA2. For example, the second predetermined concentration of the recombinant Lp-PLA2 of the kit may be zero.

A lipoprotein-associated phospholipase A2 (Lp-PLA2) kit for use with an Lp-PLA2 assay, the kit having a shelf-life of greater than 4 months, may include: a first value-assigned solution comprising a first predetermined concentration of a recombinant Lp-PLA2 in a first buffer solution, wherein the first buffer solution comprises a cholate detergent forming a plurality of micelles that stabilize the recombinant Lp-PLA2, a protein buffered matrix (e.g., BSA), a pH buffer and a preservative; and a second value-assigned solution comprising a second predetermined concentration of the recombinant Lp-PLA2 in a second buffer solution, wherein the second buffer solution comprises a cholate detergent forming a plurality of micelles that stabilize the recombinant Lp-PLA2. For example, the cholate detergent of the first and second buffer solution may comprise CHAPS. The preservative of the first and second buffer solution may comprise sodium azide.

Also described are methods of estimating the amount, activity or amount and activity of lipoprotein-associated phospholipase A2 (Lp-PLA2) from a patient sample, the method comprising: combining at least a value-assigned solution comprising a first predetermined concentration of a recombinant Lp-PLA2 in a buffer solution with an agent that interacts with Lp-PLA2 to produce a detectable first signal, wherein the buffer solution comprises a detergent forming a plurality of micelles that stabilize the recombinant Lp-PLA2; detecting the first signal; combining at least a portion of the patient sample with the agent that interacts with Lp-PLA2 to produce a detectable second signal; detecting the second signal; and assigning a value for activity, concentration or activity and concentration of Lp-PLA2 from the patient sample using the second signal. Assigning the value for an activity, concentration or activity and concentration of Lp-PLA2 from the patient sample may include calibrating the second signal based on the first signal. These methods may also include determining the validity of the assigned value by comparing the value of the first signal to a predetermined value or a predetermined range of values.

In some variations, the method may include combining a second value-assigned solution comprising a second predetermined concentration of a recombinant Lp-PLA2 in a second buffer solution with the agent that interacts with Lp-PLA2 to produce a detectable third signal, wherein the second buffer solution comprises a plurality of micelles of a detergent stabilizing the recombinant Lp-PLA2; and detecting the third signal.

In general, combining the value-assigned solution with the solution comprising the agent that interacts with Lp-PLA2 may include using a value-assigned solution that has a shelf-life of greater than 4 months. As mentioned above, the agent may be an antibody that binds to Lp-PLA2 (including a labeled antibody, e.g., conjugated to an indicator). Combining the value-assigned solution with the solution comprising the agent that interacts with Lp-PLA2 may comprise combining the value-assigned solution with the solution comprising a substrate to Lp-PLA2.

Any of the value-assigned solutions (e.g., calibrators, standards, controls, etc.) having rLp-PLA2 described herein may be specifically configured as a low-salt solution that has a long shelf-life. Such solutions may include a first detergent forming a plurality of micelles stabilizing the rLp-PLA2 (e.g., where the detergent has a concentration that is above the CMC), and a second detergent that prevents aggregation of the rLp-PLA2. The first and second detergents may be different (e.g., a cholate detergent and a polysorbate detergent) or, in some variations they may be the same detergent. For example, the detergent forming the micelles may be sufficient (e.g., at a sufficient concentration) to both form micelles and to separately prevent aggregation of the rLp-PLA2.

For example, a value-assigned solution of lipoprotein-associated phospholipase A2 (Lp-PLA2) for use with an Lp-PLA2 assay as a control, standard, calibrator or re-calibrator, the value-assigned solution having a shelf-life of greater than 4 months, may include: a first predetermined concentration of a recombinant Lp-PLA2 in a low-salt buffer solution having a salt concentration below about 1 M, wherein the low-salt buffer solution comprises a detergent (e.g., a cholate detergent such as CHAPS) forming a plurality of micelles that stabilize the recombinant Lp-PLA2. The value-assigned solution may include a second detergent to prevent aggregation of the recombinant Lp-PLA2. The second detergent comprises a non-ionic detergent, such as a polysorbate detergent (e.g., Tween 80, Tween-20, etc.). The salt in the low-salt buffer solution typically comprises a non-chaotropic salt (e.g., NaCl and an acetate salt).

For example, a value-assigned solution of lipoprotein-associated phospholipase A2 (Lp-PLA2) for use with an Lp-PLA2 assay as a control, standard, calibrator or re-calibrator, the value-assigned solution having a shelf-life of greater than 4 months, the value-assigned solution comprising: a first predetermined concentration of a recombinant Lp-PLA2 in a low-salt buffer solution having a salt concentration below about 1 M, wherein the low-salt buffer solution comprises a detergent forming a plurality of micelles that stabilize the recombinant Lp-PLA2 and a second detergent to prevent aggregation of the recombinant Lp-PLA2.

Also described herein are lipoprotein-associated phospholipase A2 (Lp-PLA2) kits for use with an Lp-PLA2 assay, the kit having a shelf-life of greater than 4 months, the kit comprising: a first value-assigned solution comprising a first predetermined concentration of a recombinant Lp-PLA2 in a first low-salt buffer solution having a salt concentration below about 1 M, wherein the first buffer solution comprises a cholate detergent forming a plurality of micelles that stabilizes the recombinant Lp-PLA2, a protein buffered matrix, a pH buffer and a preservative; and a second value-assigned solution comprising a second low-salt buffer solution having a salt concentration below about 1 M, wherein the second buffer solution comprises a plurality of micelles of the cholate detergent (e.g., CHAPS).

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a table showing retention time and recovery yield of recombinant Lp-PLA2 (rLp-PLA2). Purified rLp-PLA2 was fractionated using a newly purchased Superose-6 column and 5-25 μl of each fraction were assayed by CAM as described in the experimental section. Peak fractions were used to represent the retention time. Yields were calculated via dividing the total activity units from all fractions by the total activity units injected. Over recovery of Triton X-100 was due to the high background of the detergent in CAM assay.

FIGS. 1A-1C shows fractionation of rLp-PLA2 with a Superose-6 column. FIG. 1A shows Fractionation of rLp-PLA2 with Superose-6 column in the presence and absence of 10 mM CHAPS. Ten uL of purified rLp-PLA2 in 50 mM Tris/HCL, pH 8.0, containing 5 mM CHAPS at the concentration of 1.1 mg/ml were fractionated by a 10 cm×300 cm Sperose-6 column equilibrated in 50 mM sodium phosphate, pH 7.4 containing 100 mM sodium chloride, 2 mM EDTA and 0.02% sodium azide. Fractions were collected at 0.6 ml/tube with 0.3 mm/min flow rate. Five uL from each fraction were assayed CAM as described. FIG. 1B shows Fractionation of C-terminal His-tag rLp-PLA2 with Superose-6 column in the absence of detergents. Eleven uL of purified rLp-PLA2 with a C-terminal His-tag at 2.96 mg/ml were fractionated in the same process as in A. Forty five uL from each collected fraction were assayed by CAM and fifty uL were assayed by HisGrap-ELISA. FIG. 1C shows fractionation of purified C-terminal HIS-tag rLp-PLA2 with Superose-6 column in the presence of 10 mM CHAPS. Fifteen uL of rLp-PLA2 with a C-terminal His-tag at 0.7 mg/ml were fractionated in the same process as in IA. Fifty uL from each collected fraction were assayed by CAM and fifty uL were assayed by HisGrap-ELISA.

FIG. 2 shows a graph of the normalized activity versus time of incubation. Purified rLp-PLA2 was diluted into PBS, pH 7.54, at the final concentration of 2.9 ug/ml and the solution was incubated at 4° C. Five uL of the solution was drawn at the indicated time for the activity assay by CAM. One uL was further diluted 50 fold and 20 uL of the diluted solution were assayed for mass by PLAC. All mass and activity values were normalized against the initial value.

FIGS. 3A and 3B show the effects of detergents on the dilution in activation and activity of rLp-PLA2. FIG. 3A is a graph showing protection of rLp-PLA2 from dilution inactivation by 10 mM CHAPS. Purified rLp-PLA2 in 50 mM Tris/HCl, pH 8.0 with 10 mM CHAPS was diluted 1000 fold into PBS, pH 7.5, with (▾) and without (▪) 10 mM CHAPS at the fin al concentration of 1.3 ug/ml. Samples were incubated at 4° C. At the indicated time point, 5 uL of each sample were used to assay for activity by CAM as described. At the 15th day of the incubation, 100 uL of the enzyme mixture without detergent were withdrawn and mixed with 2 uL of 0.5 M CHAPS to obtain the final detergent concentration of 10 mM (▴). Enzymatic activities were monitored for another 10 days using the same method. FIG. 3B is a graph showing a dose dependence of detergents in the protection of the rLp-PLA2 from dilution in activation. Purified rLp-PLA2 in 50 mM Tris/HCl, pH 8.0 with 10 mM CHAPS was diluted 1340 fold at the final concentration of 1 ug/ml into 50 mM sodium phosphate, pH 7.0, containing 100 mM sodium chloride and various concentration of detergents: CHAPS (▪), sodium deoxycholate (▴), triton X-100 (▾), Digitonin (♦) and Tween-20 (). The mixtures were incubated at room temperature and the activities of the enzyme were assayed by CAM as described in the experimental section. The initial inactivation rates were obtained by linear regression analyses of the rLp-PLA2 inactivation within the incubation time from 0 to 500 minutes and plotted against the logistic values of detergent concentrations, are presented as a Lineweaver-Burke plot.

FIGS. 4A and 4B graphically show activity of the rLp-PLA2 over time of incubation and residual activity per pLp-PLA2 concentration, showing concentration effects on the dilution inactivation of rLp-PLA2. In FIG. 4A, the rLp-PLA2 in 50 mM Tris/HCL, pH 8.0, with 10 mM CHAPS was diluted into PBS, pH 7.2 at the indicated final concentrations. Activities were followed by CAM assay at the indicated time. Volumes used for assays were adjusted based on the concentration of the enzymes so that the determined activities were in the linear range. Activities were normalized to the initial values. Assay conditions were as described in the experimental section. FIG. 4B shows normalized activities on Day 10, plotted against the final concentrations of rLp-PLA2.

FIG. 5 graphically illustrates protection of rLp-PLA2 from inactivation during dilution by HDL and LDL. Purified rLp-PLA2 was diluted in 50 mM sodium phosphate buffer, pH 7.2, containing 150 mM sodium chloride and 2 mM EDTA at the final concentration of 0.5 ug/ml enzyme and incubated at 4° C. for 2 days. The experiments were carried out in the presence of various concentrations of fractionated LDL and HDL (devoid of endogenous Lp-PLA2 activity). Only the selected data are presented and the lipoprotein concentrations are indicates as that of triglyceride.

FIG. 6 illustrates the effects of chaotropic agents on the stability and activity of rLp-PLA2. The effects of different anions on the dilution inactivation of rLp-PLA2 are shown. Purified rLp-PLA2 in 50 mM Tris, pH 8.0, with 10 mM CHAPS was diluted 1000 fold to the final concentration of 1.3 ug/ml in 12.5 mM sodium phosphate, pH 7.6, containing 1 M of the indicated salt. The enzyme mixtures were incubated at 4° C. and the activities were followed by CAM assay as described, using 5 uL of the enzyme mixture. Data points were fitted with Boltzmann sigmoidal curves.

FIG. 7 shows cross-linking of rLp-PLA2. rLp-PLA2 was stored and cross-linked under different conditions as indicated. The cross-linked proteins were then resolved by SDS-PAGE and signals were detected by Western analyses.

FIGS. 8A-8D illustrate a detergent Comparison Study: Calibrator Stability, Full Panel of Detergents. The full panel of detergents used for formulate the test calibrators is shown on the Variability Chart (8A) with percent stability shown as a function of detergent identity, day of study (time point, in days), and detergent concentration relative to CMC (i.e., magnitude of the variation due to the different formulations) using the JMP 9.0.2 statistical software package. There are forty-four detergent combinations used in the formulated calibrators used in this study that vary by identity and concentrations. In addition, four lots of the standard CHAPS detergent were sourced from two different vendors (Sigma and Dojindo) and of two different grades (Sigma standard grade and BioXtra grade) were surveyed. The detergents and their published CMC values (Dojindo) are as follows: BIGCHAP (CMC: 2.9 mM), CHAPS (CMC: 8 mM), CHAPSO (CMC: 8 mM), deoxy-BIGCHAP (CMC: 1.4 mM), MEGA-8 (CMC: 58 mM), n-Dodecyl-β-D-maltoside (CMC: 0.17 mM), n-octyl-β-glucoside (CMC: 25 mM), sodium cholate (CMC: 14 mM). Except for the MEGA-8 detergent (for technical reasons related to its extremely high CMC value), the 1.00 concentration represents the detergent-specific CMC for all the other detergents surveyed. For the MEGA-8 detergent, the 1.00 concentration represents a final concentration of 34.51 mM, and the 1.68 concentration represents a final concentration of 50.00 mM. Percent stability of formulated calibrators is calculated relative to refrigerated kit calibrators. As shown in the lower right box, the green hatched line indicates target of 100% stability, and red dashed lines indicate the provisional 97%-103% calibrator stability specification used throughout these studies. Group means are shown according the legend on the right. Gauge analysis trending for percent stability is shown for detergent identity (8B), day of study (8C), and detergent concentration relative to CMC (8D). Trend line indicates the mean response by each main effect in the model. The provisional calibrator stability specification of 100%+/−3% is as described in FIG. 8A.

FIGS. 9A-9D. Detergent Comparison Study: Calibrator Precision, Full Panel of Detergents. (9A) The full panel of detergents used to formulate the test calibrators is shown on the Variability Chart with precision shown as a function of detergent identity, day of study (time point, in days), and detergent concentration relative to CMC. The general format of the Variability Chart format is as described in FIG. 8A. The grand mean of the coefficient of variation (% CV) for all samples in this study was 1.81% and is shown as a dashed line. A gray hatched line shows the target % CV of 0.00%. A lower % CV value is superior to a higher one. Group means are shown according the legend on the right. Gauge analysis trending for precision is shown for detergent identity (9B), day of study (9C), and detergent concentration relative to CMC (9D). The general format of the gauge analysis trending and trend line are as described in FIG. 8B-8D. Target % CV is as described in FIG. 9A.

FIGS. 10A-10D. Detergent Comparison Study: Calibrator Stability, CHAPS Detergent Subset. (10A) The subset of the calibrators formulated with the various CHAPS detergents (a subset of the sixteen CHAPS-formulated calibrators) is shown on the Variability Chart with percent stability as a function of detergent identity, day of study (time point, in days), and detergent concentration relative to CMC. Analysis, layout and specifications are as indicated in FIG. 8A. Gauge analysis trending for percent stability is shown for detergent identity (10B), day of study (10C), and detergent concentration relative to CMC (10D). Gauge analysis formatting is as described in FIG. 8B-1D.

FIGS. 11A-11D. Detergent Comparison Study: Student's T-test, Calibrator Stability, CHAPS Detergent Subset. Individual pairwise comparisons of means were computed using Student's T-tests using JMP 9.0.2 statistical software. Groups that are different from the selected group appear as thick gray circles. Groups that are not different from the selected group appear as thin circles. The selected group appears as thick circle. The four CHAPS concentrations tested are 2.83 mM, 4.76 mM (standard calibrator concentration), 8.00 mM (CHAPS CMC), and 13.44 mM are shown in (11A), (11B), (11C) and (11D), respectively, and shown in purple typeset. The Means Comparison report for each pair of comparisons is shown below each chart. A statistically significant difference of the mean for any given comparison is p<0.05.

FIG. 12 shows a table illustrating descriptive statistics for a detergent comparison experiment

FIG. 13 shows a Material Variation Study: Overview of the Experimental Design. The composition of the two collections of raw materials is shown as the “Red Team” and the “Blue Team”. The “Red Team” represents a standard grade of reagents, with the exception of the use of USP, Ph. Eur. (GMP) grade of water in formulating each raw material. The “Blue Team” represents a test grade of reagents, with the exception of the use of the standard (HPLC) grade of water in formulating each raw material. Individually, the indicated lots/grades of each raw material from one collection of raw materials were systematically tested in the context of the other collection of raw materials.

FIG. 14. Material Variation Study: Calibrator Stability, Each Material Substitution Tested. The full panel of raw material substitutions used for formulate the test calibrators is shown on the Variability Chart with percent stability shown as a function of each formulation condition and day of study (time point, in days). Formulation Condition #1 is the standard “Red Team” formulation which uses CHAPS lot #1 and BSA lot “A” and is boxed in red. Formulation Condition #35 standard “Blue Team” formulation which uses CHAPS lot #7 and BSA lot “F” and is boxed in light blue. The survey of individual substitution of the indicated lot of CHAPS and BSA into the context of the “Red Team” standard grade of raw materials is indicated by Conditions #4-9 and #16-20, respectively, with the appropriate comparison being Condition #1. The survey of individual substitutions of the indicated lot of CHAPS and BSA into the context of the “Blue Team” test grade of raw materials is indicated by Conditions #10-15 and #21-25, respectively, with the appropriate comparison being Condition #35. Individual substitution of indicated lot of Tris, DTT, sodium chloride, water grade, glycerol and the absence of ProClin-300 are indicated by Conditions #2-3, #26-27, #28-29, #30-31, #32-33 and #34/36, respectively. Percent stability of formulated calibrators is calculated relative to frozen kit calibrators. Red Team raw material substitutions are boxed in grey, and Blue Team raw material substitutions are boxed in light grey. CHAPS and BSA lots surveyed that are not raw materials found in the Red or Blue Team collections of raw materials are boxed separately. Conditions in which Proclin-300 are boxed as well. Arrows show representative comparisons of raw material substitution conditions to the Red and Blue team collection of raw materials, respectively. The provisional 97%-103% calibrator stability specification is indicated as in FIG. 8A.

FIGS. 15A-15B. Material Variation Study: Calibrator Stability Trending, Part 1. Gauge analysis trending for percent stability is shown for material variation formulation condition (15A) and day of study (15B). The provisional 97%-103% calibrator stability specification is indicated as in FIG. 8A.

FIGS. 16A-16C. Material Variation Study: Calibrator Stability Trending, Part 2. Gauge analysis trending for percent stability is shown for CHAPS lot number designate (16A), BSA lot number designate (16B), and the interaction of CHAPS lot number designate and BSA lot number designate (16C). The provisional 97%-103% calibrator stability specification is indicated as in FIG. 8A.

FIG. 17. Material Variation Study: Calibrator Stability, Parsed by Lots of CHAPS, BSA and Water Grade. The percent stability was plotted on the y-axis as a function of time on the x-axis. The vertical panels show material variation in the CHAPS lot usage, and the horizontal panels show material variation of the BSA lot usage. Material variation by water vendor usage is shown in traces per the legend to the right.

FIGS. 18A-18F. Material Variation Study: Calibrator Stability Trending, Part 3. Gauge analysis trending for percent stability is shown for water vendor (18A), Tris vendor (18B), DTT vendor (18C), sodium chloride vendor (18D), glycerol vendor (18E), and absence/presence of ProClin-300 (18F). The provisional 97%-103% calibrator stability specification is indicated as in FIG. 8A.

FIG. 19. Material Variation Study: Calibrator Stability, Parsed by Lots of CHAPS, BSA and Glycerol Grade. The percent stability was plotted on the y-axis as a function of time on the x-axis. The vertical panels show material variation in the CHAPS lot usage, and the horizontal panels show material variation of the BSA lot usage. Material variation by glycerol vendor usage is shown in traces per the legend to the right.

FIG. 20. Material Variation Study: Calibrator Precision, Each Material Substitution Tested. The full panel of raw material substitutions used for formulate the test calibrators is shown on the Variability Chart with precision (% CV) shown as a function of each formulation condition and day of study (time point, in days). The presentation of the results is as described in FIG. 14. The grand mean of the coefficient of variation (% CV) for all samples in this study was 1.89% (see also FIG. 22A) and is shown as a dashed line. A gray hatched line shows the target % CV of 0.00%.

FIGS. 21A-21E. Material Variation Study: Calibrator Precision, Trending. Gauge analysis trending for precision (% CV) is shown for material variation formulation condition (21A), day of study (21B), CHAPS lot number designate (21C), BSA lot number designate (21D). The presentation of the results is as described in FIG. 20. The interaction of the CHAPS lot and BSA lot is shown by the effect on standard deviation of the twelve mean % CV measurements across all timepoints (21E); see also FIG. 22B. For the standard deviation plots (E), the darker lines connect the square root of the mean weighted variance for each effect. The ovals indicate the spread in the standard deviations of the BSA CHAPS lots with the BSA lot A or F, respectively. The numbers indicate formulation conditions with reciprocal effects on the standard deviations of the mean % CV depending on BSA and CHAPS raw material combinations used.

FIGS. 22A and 22B are tables showing descriptive statistics for the material variation study described herein.

FIG. 23. Response Surface Design: The Experimental Design. A conceptual illustration of a central composite design (CCD) for three hypothetical factors is shown as three-dimensional cube (x, y, and z) enclosed by a sphere. The imbedded factorial design with center point is shown are indicated by the vertices of a cube with a center point. The center point is located in the exact center of both the cube and the sphere. The group of “star points” (also known as axial points [see inset] are indicated by “a” and “A”) reside on the surface of the sphere. The star points are at some distance from the center based on the properties desired for the design and the number of factors in the design. The star points establish new extremes for the low and high settings for all factors and are surveyed in conjunction with the midpoint concentrations of the other effectors. For this specific, rotatable CCD design with four effectors, the axial concentrations are twice the distance from the low/high factorial level to the midpoint. The five effector concentrations for each of the four effectors in this experimental design are shown in the lower right corner. The low axial, low factorial, center point, high factorial and high axial concentration are coded by a, −, 0, +, and A, respectively. Thus, the midpoint concentration for all four effectors would be represented by “0000”. The effector concentrations circled are the standard concentrations currently used in the calibrator matrix (Tris, DTT, CHAPS) or the lower/upper specification limits for pH, as described in the MP-21090 document.

FIG. 24 is a table showing the response surface design for effector concentration as described below.

FIG. 25. Response Surface Design: Calibrator Stability as a Function of Raw Material Concentration. The percent stability the twenty six calibrators formulated in this study are shown as a function of CHAPS concentration, DTT concentration, buffer pH, buffer concentration and time (in days). Percent stability is shown as a percentage of the OD on the indicated time point of the OD on Day 0 of the study. The nine time points shown were taken on Day 3, 8, 14, 30, 60, 90, 120, 150, and 180. The Day 0 time point, by definition, is set to 100%. The conditions are numbered as in Table 3. Note that the midpoint formulation is intentionally duplicated (conditions #13/#14) as part of the designed experiment.

FIG. 26. Response Surface Design: Absolute Value of Calibrator Stability Differential Relative to Target. The absolute value of the percent stability differential relative to 100% stability is shown as a function of formulation condition number. Formulation condition number is as described in FIG. 24. The gray hatched line indicates the 100% target (that is, 0% differential from target) and the red hatched line indicates the absolute value of the provisional +/−3% specification. The stability data points shown in blue are from the formulation condition with the low axial concentration (0.90 mM) of CHAPS surveyed. The stability data points shown in blue are from the formulation condition with the low axial concentration (0.05 mM) of DTT surveyed. The stability data points shown in orange are from the formulation condition with the low axial concentration of protons (pH 8.18).

FIGS. 27A-27E. Response Surface Design: Calibrator Stability, Trending, as a Function of Raw Material Concentration. Gauge analysis trending for percent stability is shown for CHAPS concentration (27A), DTT concentration (27B), buffer pH (27C), buffer concentration (27D), and time in days (27E). The provisional 97%-103% calibrator stability specification is indicated as in FIG. 8A.

FIGS. 28A-28C. Response Surface Design Study: Refined Model for Calibrator “Maximum Stability”. The analytics of the refined modeling of percent stability using a response surface model, including time, using JMP 9.0.2 is shown in (28A). The parameter estimates including the p values are shown in (28B). Statistical significance is p<0.05. The “Prediction Profiler” was set to maximize stability for each of the twenty-six formulations in the model in (28C). Accordingly, the “Response Limit” for “Stability” was set to “Maximize” and the Prediction Profiler was set to “Maximize Desirability”. Condition #12 (the axial low CHAPS concentration) was excluded from the model at each time point. The effector concentrations shown are the predicted optimal effector concentrations calculated to achieve a maximal stability response. Within each individual plot, the line within the plots (i.e., the prediction trace) show how the predicted value changes as a function of the value of an individual X variable. The 95% confidence interval for the predicted values is shown by a dotted curve surrounding the prediction trace (for continuous variables, e.g., pH). The bottom row has a plot for each factor, showing its desirability trace. The profiler also contains a Desirability column, which graphs desirability on a scale from 0 to 1 and has an adjustable desirability function for each y variable. The overall desirability measure is on the left of the desirability traces.

FIG. 29. Response Surface Design Study: Raw Materials Concentration-Dependent Effects on Stability. The percent stability was plotted on the y-axis as a function of time on the x-axis. The vertical panels show increasing CHAPS concentration from left to right, and the horizontal panels show increasing DTT concentration from top to bottom. The five pH's tested are shown as colored traces per the legend located to the right.

FIGS. 30A and 30B show a table of the descriptive statistics of calibrator analyte values/precision for a response surface design, as discussed herein.

FIGS. 31A-31B. Response Surface Design Study: Calibrator Precision, as a Function of Raw Material Concentration. The precision of the twenty-six calibrators formulated in this study are shown as a function of CHAPS concentration, DTT concentration, buffer pH, buffer concentration and time (in days). The general format of the Variability Chart format is as described in FIG. 25. The grand mean of the coefficient of variation (% CV) for all samples in this study was 2.22% (See FIG. 30A) and is shown as a dashed line. A gray hatched line shows the target % CV of 0.00%.

FIGS. 32A-32H. Response Surface Design Study: Calibrator Precision, Trending, by Raw Material Concentration. The precision results were trended by CHAPS concentration (32A), DTT concentration (32B), buffer pH (32C) and buffer concentration (32D). The grand mean % CV and target % CV are as described in FIG. 20. The mean standard deviations of the % CV's are trended for CHAPS concentration (32E), DTT concentration (32F), buffer pH (32G) and buffer concentration (32H). The analysis is analogous to that described in FIG. 21E.

FIG. 33 is a table of a buffer/BSA survey as discussed herein.

FIG. 34 is a table showing descriptive statistics of calibrator stability at two storage temperatures as discussed herein.

FIG. 35. Buffer/BSA Survey: Calibrator Stability, Stored Frozen at −70 Celsius. The stability results for the twenty-six different permutations of the buffer composition/process and BSA survey are shown as a variability chart for the samples stored frozen at −70° Celsius. Percent Stability is shown as a function of buffer composition, pre-pH process, final pH process, buffer concentration, BSA lot number and time on stability (in days). Percent stability is shown as a percentage of the OD on the indicated time point of the OD on Day 0 of the study. The eight timepoints were taken on Days 1, 4, 7, 30, 45, 60; 90, and 120. The Day 0 time point, by definition, is set to 100%. The conditions are numbered as in FIG. 34.

FIG. 36. Buffer/BSA Survey: Buffer/BSA Survey: Calibrator Stability, Stored at Refrigeration Temperature. The stability results for the twenty-six different permutations of the buffer composition/process and BSA survey are shown as a variability chart for the samples stored at 4-8° Celsius. Layout and analysis is as described in FIG. 35.

FIGS. 37A-37G. Buffer/BSA Survey: Calibrator Stability, Trending, Stored Frozen at −70 Celsius. The trending of the percent stability results using gauge analysis for the twenty-six different permutations of the buffer composition/process and BSA survey is shown for the samples stored frozen at −70° Celsius. Gauge analysis is shown as a function of condition number (37A), time (37B), buffer composition (37C), pre-pH process/pH (37D), final pH (37E), buffer concentration (37F), and BSA lot number (37G). Layout is as described in FIG. 35.

FIGS. 38A-38G. Buffer/BSA Survey: Calibrator Stability, Trending, Stored at Refrigeration Temperature. The trending of the percent stability results using gauge analysis for the twenty-six different permutations of the buffer composition/process and BSA survey is shown for the samples stored refrigerated at 4-8° Celsius. Gauge analysis is shown as a function of condition number (38A), time (38B), buffer composition (38C), pre-pH process/pH (38D), final pH (38E), buffer concentration (38F), and BSA lot number (38G). Layout is as described in FIG. 35.

FIG. 39 is a table showing descriptive statistics of OD and imprecision at two storage temperatures, as described herein.

FIG. 40. Buffer/BSA Survey: Calibrator Precision, Stored Frozen at −70° Celsius. The precision results for the twenty-six different permutations of the buffer composition/process and BSA survey are shown as a variability chart for the samples stored frozen at −70° Celsius. Precision is determined using all nine timepoints, including Day 0. Layout is as described in FIG. 35.

FIGS. 41A-41G. Buffer/BSA Survey: Calibrator Precision, Trending, Stored Frozen at −70 Celsius. The trending of the precision results using gauge analysis for the twenty-six different permutations of the buffer composition/process and BSA survey is shown for the samples stored frozen at −70° Celsius. Gauge analysis is shown as a function of condition number (41A), time (41B), buffer composition (41C), pre-pH process/pH (41D), final pH (41E), buffer concentration (41F), and BSA lot number (41G). Layout is as described in FIG. 35.

FIG. 42. Buffer/BSA Survey: Calibrator Precision, Stored at Refrigeration Temperature. The precision results for the twenty-six different permutations of the buffer composition/process and BSA survey are shown as a variability chart for the samples stored refrigerated at 4-8° Celsius. Analysis is as described in FIG. 40, and layout is as described in FIG. 35.

FIGS. 43A-43G. Buffer/BSA Survey: Calibrator Precision, Trending, Stored at Refrigeration Temperature. The trending of the precision results using gauge analysis for the twenty-six different permutations of the buffer composition/process and BSA survey is shown for the samples stored refrigerated at 4-8° Celsius. Gauge analysis is shown as a function of condition number (43A), time (43B), buffer composition (43C), pre-pH process/pH (43D), final pH (43E), buffer concentration (43F), and BSA lot number (43G). Layout is as described in FIG. 35.

FIGS. 44A and 44B. Buffer/BSA Survey: Calibrator Stability/Precision, Trending, at Two Storage Temperatures. The percent stability (44A) and precision (44B) of the set of twenty-six calibrators is compared for the frozen (−70 Celsius) and refrigerated (+4 Celsius) storage temperatures.

FIGS. 45A and 45B. Robust Design: Transmission of Variation from Inputs to Outputs. A textbook example of Robust Design Principles is shown in (45A). The non-linear relationship between the x inputs and the y outputs is shown by the line and indicated by a green arrow and typeset. The variation of the inputs on the x-axis is indicated by red arrows and typeset. The variation of the outputs on the y-axis is indicated by blue arrows and typeset. The non-linear relationship between the percent stability and CHAPS concentration is shown in (45B).

### DETAILED DESCRIPTION

In general, described herein are compositions, kits, assays, including recombinant Lp-PLA2 calibrations solutions and methods of making an using them. In particular, described herein are calibration solutions having a predetermined amount of recombinant Lp-PLA2 (rLp-PLA2) that is stabilized by a plurality of micelles formed of a detergent, so that the recombinant Lp-PLA2 retains activity and antigenicity (e.g., to an antibody to a native Lp-PLA2) for an extended period of time (e.g., greater than 4 months, greater than 5 months, greater than 6 months, greater than 7 months, greater than 8 months, greater than 9 months, greater than 10 months, greater than 11 months, greater than 12 month, greater than 13 months, greater than 14 months, greater than 15 months, greater than 16 months, greater than 17 months, greater than 18 months, etc.). In some variations, described herein are calibration buffers, methods of making them, and kits and assays including them that are low-salt calibrations buffers (e.g., having less than 1 M salt). Such low-salt calibration buffers may include a second detergent that prevents aggregation of the rLp-PLA2.

The calibration solutions described herein are an advantage over existing calibration solutions for Lp-PLA2 and particularly rLp-PLA2, which typically have a short (e.g., less than 4 months) shelf life before activity of the rLp-PLA2 in the calibration solution deteriorates. Dilution of rLp-PLA2 in the absence of detergents results in irreversible gradual inactivation of the enzyme. Even in the presence of a detergent, deterioration occurs over a comparable time scale (e.g., between 4-6 months). The monomeric rLp-PLA2 may expose its hydrophobic interfacial binding region or substrate binding compartment to water and cause structural collapsing of the enzyme. Further, once activity of the rLp-PLA2 is lost, it cannot typically be recovered.

As described herein, certain detergents, if used to form micelles, can fully protect the enzyme from the inactivation, but cannot recover the activity of the inactivated enzyme. Further, purified recombinant lipoprotein-associated phospholipase A2 (rLp-PLA2) expressed in HEK293 cells has a propensity to form oligomers in the absence of detergents or lipids by chemical cross-linking. These observations suggest that the Lp-PLA2 may form non-covalent oligomers in the absence of lipids or detergents which serve to block access for the aqueous solvent to the hydrophobic substrate binding site and therefore prevents structural collapsing. Further, dilution inactivation of the enzyme can be prevented in the presence of LDL or HDL suggesting that Lp-PLA2 association with lipoprotein particles (LDL and HDL) is necessary for Lp-PLA2 to maintain its enzymatic activity in human plasma.

Abbreviations used herein include: BSA (bovine serum albumin); CHAPS, (3-[(3-Cholamidopropyl) dimethylammonio]-1-propanesulfonate); CMC (critical micelle concentration); DMPC (1,2-dimyristoyl-sn-glycerol-3-phosphocholine); DTT (Dithiothreitol; EDTA, ethylenediamine tetraacetic acid); EGS (Ethylene glycol bis[succinimidyl]succinate); ELISA (Enzyme-Linked Immuno Sorbent Assay); FBS (fetal bovine serum); HDL (high density lipoprotein); HEPES (4-(2-hydroxyethyl)-1-piperazineethanesulfonic acid); LDL (low density lipoprotein); MES (4-Morpholineethanesulfonic acid); PBS (phosphate buffered saline); PCR (polymerase chain reaction); SDS-PAGE (sodium dodecyl sulfate-polyacrylamide gel electrophoresis); SNS (sodium 1-nonane sulfonate); TBS (Tris buffered saline); TCEP (tris(2-carboxyethyl)phosphine hydrochloride); TMB (3.3′, 5.5′-tetramethylbenzidine); Tris (tris(hydroxymethyl)aminomethane).

Lipoprotein-associated phospholipase A₂ (Lp-PLA₂) is a Ca²⁺ independent plasma group VII lipase (Lp-PLA₂G7) bearing the structure similarity with other members of the phospholipase superfamily. The pathological roles of Lp-PLA₂ in cardiovascular diseases (CVD) are presumably attributed to the generation of inflammatory hydrolysis products, lysophosphatidyl cholines and oxidized free fatty acids. The majority of the circulating human Lp-PLA₂ in blood is synthesized by macrophages and the matured enzyme is a 45-50 kD glycosylated protein. Normally, the secreted enzyme in the plasma has been shown to associate with high density lipoproteins (HDL) and low density lipoproteins (LDL) in the ratio of about 1:2. Clinical studies have suggested that the pathogenicity of Lp-PLA₂ may be affected by the pattern of lipoprotein affiliation (10) and that the ratio of Lp-PLA₂ in lipoproteins may affect the enzymatic activity and determine its physio-pathological functions in humans. Recent publication has been shown that the composition of the cell membrane or lipid vehicles affects the association of Lp-PLA₂ and its activity. Further, it has been reported that Lp-PLA₂ can migrate between lipoproteins and it has been hypothesized that HDL may act as a transport system distributing Lp-PLA₂ between LDL particles. Therefore understanding the complex interactions between Lp-PLA₂ and lipids will be important in the design of diagnostic devices and enzyme-modulating therapeutics.

The amino acid residues of Lp-PLA₂ that are involved in the interaction with lipoproteins have been mapped out by mutagenesis and peptide amide hydrogen-deuterium exchange mass spectrometry (DXMS). Interestingly, the mapped residues are shown to compose parts of the interfacial binding region of the enzyme identified by x-ray diffraction studies of the crystal structure. The majority of the amino acid residues consisting of the interfacial binding region are very hydrophobic. It can be expected that the exposition of this interfacial binding region to aqueous phase will cause high energy potential and, therefore, induce instability of the protein. Thus, it is likely that the enzyme must have a mechanism to protect its hydrophobic region once released from the chaperon. recombinant Lp-PLA₂ may be expressed in HEK293 in order to study the interaction between the enzyme and lipids or detergents.

Materials.

1-myristoyl-2-(4-nitrophenylsuccinyl)-sn-glycero-3-phosphocholine (14:0 NPSPC) was purchased from Avanti Polar Lipids, Inc. (Alabaster, Ala.). The 10×300 mm Superose-6 column was manufactured by GE Healthcare Life Sciences (Piscataway, N.J.). Rabbit anti-Lp-PLA₂ polyclonal antibodies were originally obtained from GlaxoSmithKline and also purchased from Cayman Chemicals (Ann Arbor, Mich.). Apolipoproteins were acquired from Biodesign (Saco, Me.) or Lee BioSolutions (St. Louis, Mo.). Both recombinant and lipid-free human serum albumins (HSA) were obtained from Sigma-Aldrich (St. Louis, Mo.). PLAC Test and the Colorimetric Activity Method (CAM) assay kit for the quantitation of Lp-PLA₂ are the products of diaDexus Inc. Recombinant Lp-PLA₂ and C-terminal His-tag Lp-PLA₂ were also made by diaDexus Inc. as the components of PLAC test kit. Other equipment or reagents were indicated in the text.

SDS-PAGE, Western Blotting and Protein Concentration Determination.

All SDS-PAGE were performed by using 4-12% Bis-Tris gradient gels (Invitrogen, San Diego). Gels were blotted on to nitrocellular membranes in a buffer (pH 7.5, containing 25 mM Bicine, 25 mM Bis-Tris, 1 mM EDTA and 0.05 mM chlorobutanol) for 1 hr at 50 volts. Western blots were analyzed by using rabbit anti-Lp-PLA₂ polyclonal antibody or as indicated in the figures. All protein concentrations were determined by using either micro BCA or modified Bradford protein assays (Pierce Biotechnology) following the manufacturer's protocols. Both assays gave similar results for rLp-PLA₂.

HisGrap-Enzyme-Linked ImmunoSorbent Assay (HisGrap-ELISA) and PLAC Test Assay.

For HisGrap-ELISA, chromatography fractions were loaded and incubated in 96-well HisGrap nickel coated plates (Pierce Biotech, Rockford, Ill.) overnight with shaking. Plates were washed with 300 μl/well TBS, pH 7.4, containing 0.05% Tween-20 (TBS/T) for 6 times and incubated with 100 μl of primary rabbit polyclonal anti-Lp-PLA₂ antibody at 1 μg/ml each in the same TBS/T buffer containing 3% BSA and 0.1% Proclin-300 for 3 hr at the room temperature. The plates were then washed as described before with the same TBS/T buffer and further incubated with 100 μl of the secondary antibody (goat anti-rabbit, Jackson ImmunoResearch Laboratories, West Grove, Pa.,) labeled with horseradish peroxidase (HRP) diluted at 1:15,000 in the same TBS/T/BSA buffer for 1 hr. The plates were further washed 9 times with 300 μl/well of the same TBS/T buffer and incubated with 100 μl of TMB substrate for 5-20 minutes at the room temperature in dark. The reactions were stopped with 100 μl/well of 1 M HCl and concentrations were determined by reading of the plate in a SPECTRAmax M5 plate reader at 450 nm (Applied Biosystems, Foster City, Calif.).

For PLAC Test, briefly 1-40 μl (depending on the concentration) of each sample containing rLp-PLA₂ were applied onto the assay plate wells and the plate was incubated for 10 minutes at room temperature. Two hundred micro liters of the anti-rLp-PLA₂ antibody-HRP conjugate solution were added to each well and the plate was incubated at room temperature for 3 hr without sealing. The plate was then washed with TBS/T buffer for 4 times and incubated with 100 μl of TMB substrate solution for 20 minutes at the room temperature in dark. The reaction was stopped by adding 100 μl of 1 M HCl each well and concentrations were determined by reading of the plate in a SPECTRAmax M5 plate reader at 450 nm.

Enzyme Kinetic Assay and Analysis.

All of recombinant Lp-PLA₂ (rLp-PLA₂) enzyme kinetic assays in the study were carried out by using the CAM assay kit developed by diaDexus, Inc. Basically, in a 96-well plate, reactions were started by adding 110-134 μl of the reaction buffer to each well containing 1-25 μl of Lp-PLA₂ samples according to the protocol by the manufacturer. The volumes of enzyme and reaction buffer were depended on the individual experiment. The reactions were followed at OD405 nm (absorbance) in a SPECTRAmax M5 plate reader and the steady state reaction rates of the first 3 or 5 minutes depending on the experiments were averaged. The data were processed and analyzed by using Microsoft Excel and GraphPad Prism (version 4).

Chemical Cross-Linking of rLp-PLA₂.

Purified rLp-PLA2 in 50 mM Tris, pH 8.0, with and without 10 mM CHAPS was diluted 1340 or 1460 fold to the final concentration of 1.0 μg/ml in 50 mM sodium phosphate with and without 10 mM CHAPS or 1% Tween-20, pH 7.6, containing 100 mM sodium chloride and 3 mM EGS. The mixtures were incubated at room temperature for 45 min and ethanol amine was added to the final concentration of 0.5 M to stop the reactions. The mixtures were then concentrated about 10 fold through a 20-kD cutoff iCON concentrator. Thirty μl of each sample were mixed with 10 of 4-fold SDS-PAGE loading buffer containing 200 mM DTT and 20 mM TCEP and incubated at 60° C. for 15 minutes and subjected to electrophoresis.

FPLC Fractionation.

Fractionation chromatography was carried out on an Akta10 or Akta100 by using a 10 mm×300 mm Superose-6 column at room temperature with the flow rate of 0.3 ml per minute. Three different buffer systems (A: 50 mM sodium phosphate, pH 7.4, containing 100 mM NaCl, 2 mM EDTA and 0.01% sodium azide; B: PBS, pH 7.2; C: 50 mM Tris/HCl, pH 8.0) were used and no significant difference was observed. Fifty to two hundred μl of samples were injected per run depending on the Lp-PLA2 concentrations of the samples after the column was equilibrated with the running buffer. Fraction collection was started at 21 minutes (the column void volume) after the sample injection and the collection volume was 0.6 ml/tube.

Preparation of LDL and HDL Lipoproteins Devoid of Lp-PLA2 Enzymatic Activity.

Concentrated human LDL and HDL were purchased from Lee BioSolutions in St. Louis. According to the manufacturer, LDL and HDL were prepared from fresh human plasma by undisclosed precipitation methods. Both the LDL and HDL showed one major band by Helena lipoprotein cellulose acetate electrophoresis. Characterization indicated that triglyceride/cholesterol ratios were 0.86 and 0.40 for LDL and HDL respectively. The lipoproteins were stored at −40° C. and shipped on dry ice. The purchased lipoproteins were thawed and subjected to inactivation by incubation with 20 mM Pefabloc SC (Roche Applied Science, Indianapolis) in PBS, pH 7.2, at 4° C. overnight. The Pefabloc SC inactivated lipoproteins were then dialyzed extensively with a 10 kD cutoff membrane in 1000 fold volume excess of buffer containing 50 mM phosphate, pH 7.2, and 150 mM sodium chloride with 3 exchanges at 4° C. The inactivated lipoproteins were found to have less than 10% of the original endogenous Lp-PLA2 activity by the CAM assay. Both lipoproteins were further diluted to the desired concentrations before used in each experiment.

Results

Association of Lp-PLA₂ with Detergent Micelles.

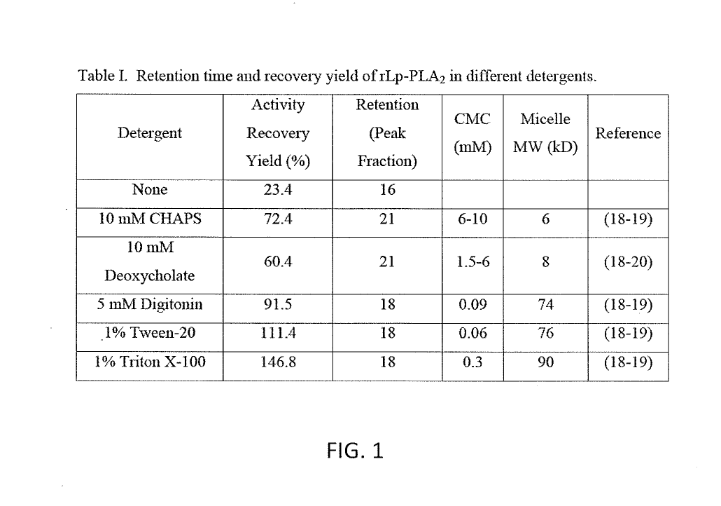

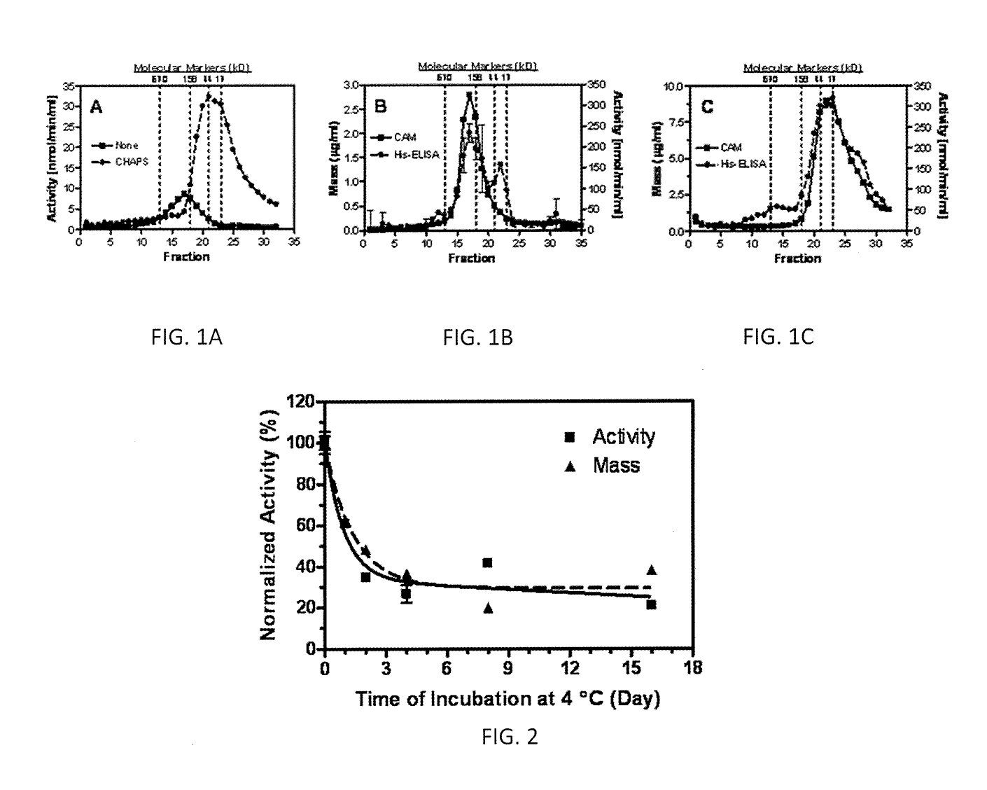

To estimate the molecular size of the rLp-PLA₂ expressed in HEK293 cells, the purified enzyme was subjected to fractionation by a 10×300 mm Superose-6 column in the presence and absence of 10 mM CHAPS. The results indicated that the same enzyme was eluted very differently under the various conditions (FIG. 1A). According to the molecular weight reference, rLp-PLA₂ was eluted between the chicken ovalbumin (44 kD) and horse myoglobin (17 kD) in the presence of 10 mM CHAPS and between the bovine thyroglobulin (670 kD) and bovine Ig-globulin (158 kD) in absence of the detergent (chromatography of molecular markers not shown). The expected molecular weight of Lp-PLA₂, not including the glycosylation oligosaccharide chains, is about 48 kD. To further understand the retention time shift, we resolved the enzyme by the same procedure with different detergents. The results showed that the column retained rLp-PLA2 differently with different detergents (FIG. 1). Detergents with larger micelle molecular weight eluted rLp-PLA2 earlier from the column. This indicates the association of rLp-PLA2 with the micelles of the detergents. However, the molecular size of the rLp-PLA2 in the absence of the detergents seems larger than that of the complex of enzyme and detergent micelles tested. This suggests a possibility that the enzyme may form oligomeric structures or aggregate in the absence of detergents. We also fractionated the unpurified rLp-PLA2 from the cell cultural supernatant of HEK293 and it gave the same results as the purified enzyme under the same conditions (results not shown). Another observation was that the recovery yield based on the CAM enzymatic assay was much lower when rLp-PLA2 was fractionated in the absence of detergents (FIG. 1). In the absence of detergents, only about 23% of rLp-PLA2 activities were recovered compared to 60-146% recovery in the presence of detergents. To investigate the lost rLp-PLA2 in the absence of detergents, purified rLp-PLA2 with a His-tag at the C-terminal was subjected to fractionation and the fractions were assayed by both the CAM assay and the His-ELISA using rabbit anti-Lp-PLA2 polyclonal antibody. When rLp-PLA2 was fractionated in the absence of detergents, the results indicated that two mass peaks (fraction 16-18 and 21-23) were shown by the His-ELISA but only one activity peak (fraction 16-18) was seen by the CAM assay (FIG. 1B). That is, the lower molecular weight mass peak (fraction 21-23) contained no enzymatic activity. However, when the enzyme was fractionated in the presence of 10 mM CHAPS in the same buffer, no mass or enzymatic activity at fraction 16-18 was seen but both mass and enzymatic activity were detected at the fraction 21-23 (FIG. 1C). This suggests that the lower molecular weight peak (fraction 21-23), which probably comes from the higher molecular weight peak (fraction 16-18), losses its activity irreversibly in the absence of detergents. In the presence of detergents, rLp-PLA2 is probably deoligomerized and stabilized by the formation of the complex with detergent micelles.

Inactivation of rLp-PLA2 by Dilution in the Absence of Detergents.

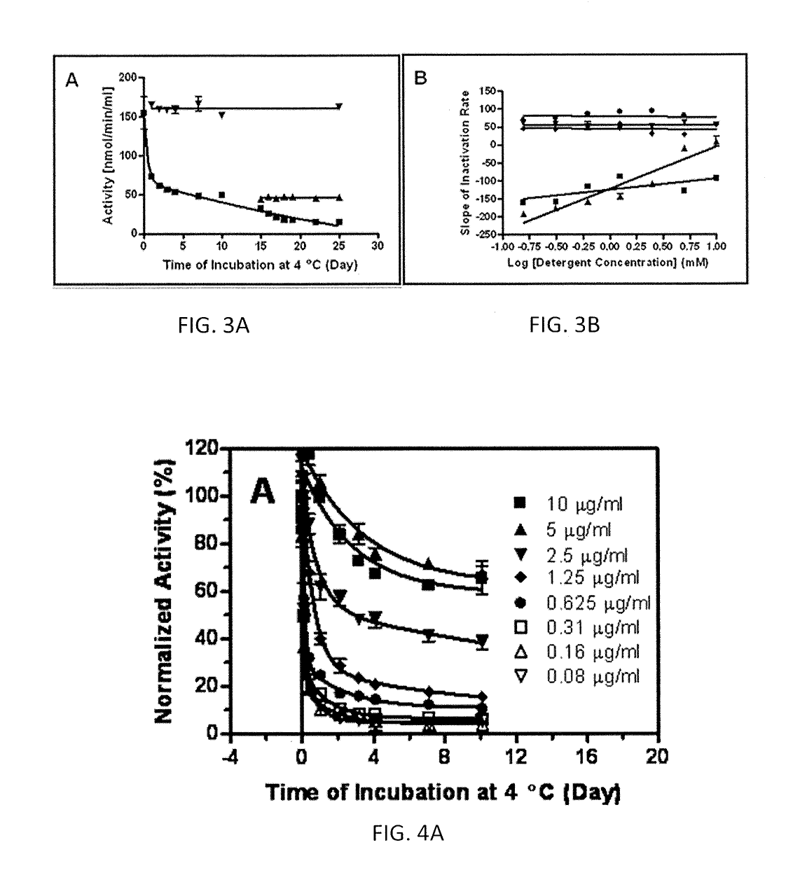

It was found that freshly prepared rLp-PLA2 stored in the presence or absence of detergents had no difference in specific activity when assayed with CAM (results not shown). However, the enzyme stored in the absence of detergents at 4° C. lost its activity faster, especially when the concentration was low (results not shown). To further investigate the decrease of rLp-PLA2 specific activity in the absence of detergents, the enzyme was subjected to dilution to the final concentration between 1-3 μg/ml in PBS, pH 7.2, and the changes of the enzymatic activity and immuno-reactive mass were followed. The immuno-reactive mass of Lp-PLA2 was quantified by using the PLAC kits that only recognized the non-denatured form of the enzyme (conformational). FIG. 3 shows that the enzyme gradually lost its activity and immuno-reactive mass in two phases. Upon dilution, the enzymatic activity and the immuno-reactive mass had a sharp decline phase (about 1-2 days of incubation at 4° C.) and then the inactivation rate decreased and transferred to a slower phase (FIG. 2). The final normalized losses in both activity and immuno-reactive mass were in the range of 50-75% at the fifteenth day of incubation. Actually, for each reaction, the inactivation rates and final losses of the enzymatic activity and immuno-reactive mass varied with different experimental conditions depending on the final diluted enzyme concentration (see the following experiments), the storage conditions of the enzyme, the dilution buffer components and incubation temperature, etc.

The Effects of Detergents on the Activity of rLp-PLA2.

The effects of detergents on the dilution inactivation of rLp-PLA2 were investigated. When 10 mM CHAPS was included in the dilution buffer, no inactivation was observed for the diluted rLp-PLA2 at 1 μg/ml (FIG. 3A). However, the addition of 10 mM CHAPS into the inactivated enzymes only recovered a very small portion of the lost activity but it did prevent the enzyme from further inactivation during the extended incubation (FIG. 3A). In addition to CHAPS, several other non-ionic detergents, such as Tween-20, Triton X-100 and digitonin, were also found protective in the dilution inactivation of rLp-PLA2 (data not shown). Detergents with high CMC were less effective than those with lower CMC. In an experiment of dilution inactivation for rLp-PLA2, the diluted enzyme was incubated in buffers containing variable detergent concentrations from 0.15 mM to 10 mM. The rate of enzyme inactivation was found to be concentration dependent for CHAPS (CMC=6 mM) and deoxycholate (CMC=1.5 mM) but not for Triton X-100 (CMC=0.3 mM), Digitonin (CMC=0.09 mM) and Tween-20 (CMC=0.06 mM) (FIG. 3B). This suggests that detergent micelles, instead of monomeric detergent, are the stabilizer of rLp-PLA2 molecule.

The Effects of the Protein Concentration on the Activity of rLp-PLA2.

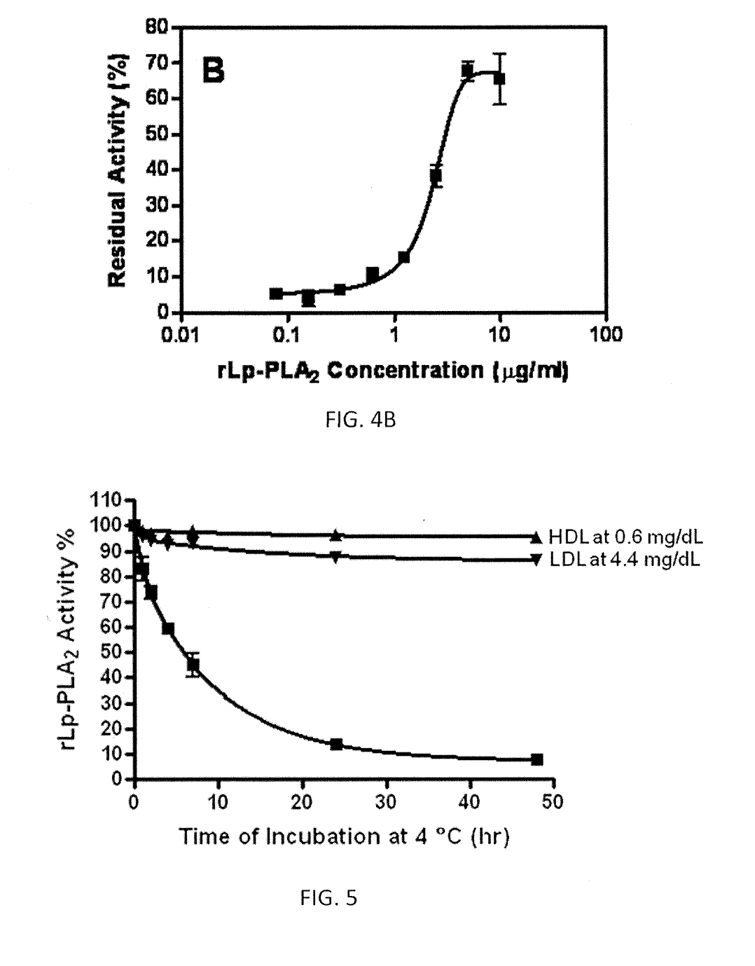

At high concentrations (>0.5 mg/ml), rLp-PLA2 is fairly stable even in the absence of detergents (observation not shown). In the dilution inactivation of the recombinant Lp-PLA2, the inactivation rates are dependent on the final diluted concentration of the enzyme. The concentration effect on the rLp-PLA2 dilution inactivation is illustrated in FIG. 4A. The rate and final loss of the rLp-PLA2 inactivation upon dilution varied in the enzyme concentration range of 0.6-5 μg/ml. The inactivation rates became relatively independent of final enzyme concentrations at both ends of the above concentration range. This can be better demonstrated by plotting the residual residue percentage of the rLp-PLA2 activity after the enzyme was diluted and incubated at 4° C. for ten days against the protein concentrations (FIG. 4B). In the logistic scale of concentration, it can be fitted into a sigmoidal curve. There is a sensitive range between 1 and 5 μg/ml. The saturation at both concentration ends may indicate that there is a dynamic equilibrium between the stable and unstable forms of rLp-PLA2, which shifts depending on the concentration of the enzyme. Since the inactivation is due to structural disruption by solvent and irreversible, it should be a reaction of first order kinetics, that is, concentration independent. When the enzyme concentration decreases to a certain level, the equilibrium is shifted to the unstable form and then the irreversible inactivation rate becomes concentration independent. When the concentration of rLp-PLA2 increases, the rate of inactivation is reduced due to the equilibrium shifting to the stable form of the enzyme. Most likely, the stable and unstable forms of Lp-PLA2 should represent the oligomerized and the dissociated enzyme respectively since the dilution usually causes dissociation and vice versa.

Protection of rLp-PLA2 Activity by Lipoproteins.

Lp-PLA2 protein has been shown to associate with LDL and HDL in human plasma (9). Experiments were designed to reveal if LDL and HDL would prevent rLp-PLA2 from the inactivation during the dilution into non-detergent containing buffers. Purified rLp-PLA2 was diluted in 50 mM sodium phosphate buffer, pH 7.2, containing 150 mM sodium chloride and 2 mM EDTA at the final concentration of 0.5 μg/ml enzyme and incubated at 4° C. for 2 days. The experiments were carried out in the presence of various concentrations of fractionated LDL and HDL (devoid of endogenous Lp-PLA2 activity). It was indeed found that the dilution inactivation of rLp-PLA2 could be averted in the presence of either LDL or HDL particles. FIG. 5 shows that human LDL or HDL at concentrations as low as 1.4 and 0.14 mg/dL of triglyceride respectively fully protected the rLp-PLA2 activity during the dilution in the phosphate buffer. No significant activity losses were observed after the two day period of incubation at 4° C. in the LDL or HDL containing buffer while more than 90% of the original activity vanished in the control buffer. However, unexpectedly higher concentrations of LDL or HDL reduced the protection capability possibly due to the proteolysis of the recombinant enzyme (data not shown).

The Effects of Chaotropic Agents on the Activity of rLp-PLA2.

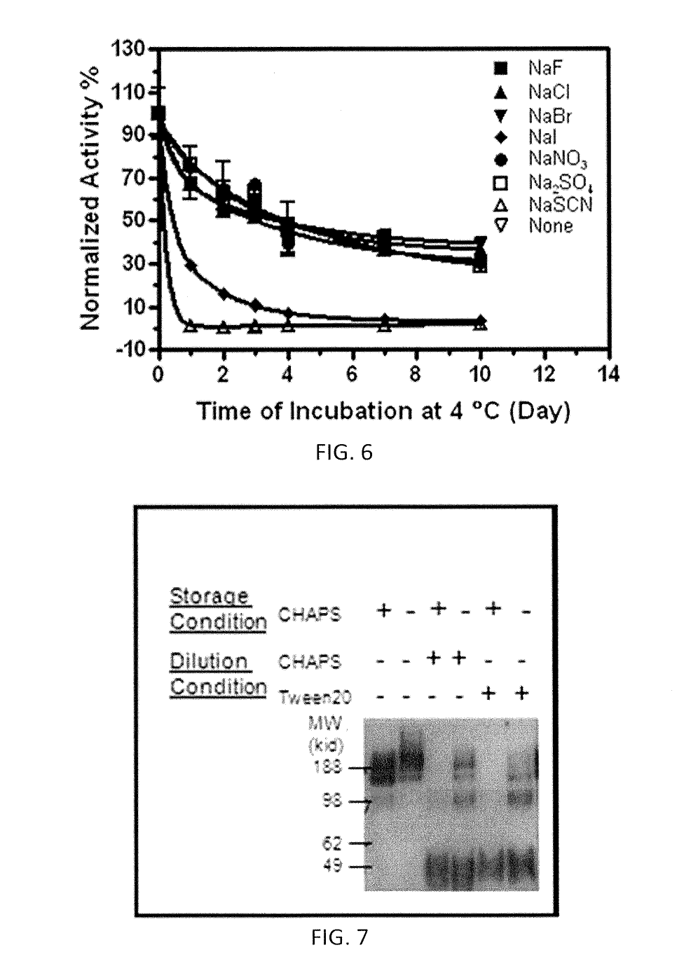

According to the gel permeation experiments, detergents could reduce the molecular weight of rLp-PLA2 and stabilize its activity. To investigate the connection between the deoligomerization and stabilization effects of detergents, rLp-PLA2 was diluted and incubated at 4° C. in the presence of 1 M sodium salts of fluoride, bromide, chloride, iodide, nitrate, sulfate (0.5 M) and thiocyanate. While detergents were found to stabilize rLp-PLA2, anions destabilizing protein-protein interactions, such as SCN⁻¹ or I⁻¹, were found to promote the inactivation of the enzyme. The inactivation of the diluted rLp-PLA2 during the incubation at 4° C. was significantly accelerated by including 1 M of NaSCN or Nal in the incubation buffer (FIG. 6). This is not due to the added sodium salt concentration because no other salts had effects on the stability of the enzyme. None of the above chemicals (up to 1 M) was found inhibitory to the enzymatic activity of rLp-PLA2 either (results not shown). The experiment suggests that the protein-protein interaction breaker such as SCN⁻¹ or I⁻¹ actually destabilizes rLp-PLA2. It can be inferred that rLp-PLA2 tenders to form a dimer or oligomers during the incubation but, if the self-interaction is prevented or interrupted by chaotropic agents, the monomeric enzyme will be denatured, possibly due to exposure of the hydrophobic substrate binding site to aqueous solvents.

Chemical Cross-Linking of rLp-PLA2.

To further confirm the formation of the oligomeric rLp-PLA₂ during dilution, the highly purified enzyme was diluted into buffers containing a chemical cross-linker, ethylene glycol bis[succinimidylsuccinate] (EGS), with and without detergents. FIG. 7 shows the results of the cross-linking experiment. First of all, when rLp-PLA2 was diluted to the final concentration of 1 μg/ml in the absence of detergents, only oligomers with molecular weight >98 kD were detected on the Western Blot by rabbit anti-Lp-PLA2 antibody. No monomeric (48 kD) and only a low amount of dimeric (98 kD) rLp-PLA2 were seen. Second, the extent of rLp-PLA2 oligomerization observed was different when stored at different conditions. Enzyme stored in buffer containing 5 mM CHAPS had a lower oligomerized molecular weight than enzyme stored in the detergent-free condition although both were diluted into the same cross-linking buffer at the same final concentration. Third, in the presence of 10 mM CHAPS (or 1% Tween-20, data not shown), the majority of rLp-PLA2 stayed monomeric after cross-linked by EGS. Again, the enzyme stored in the presence of 5 mM CHAPS was almost free of oligomeric bands when cross-linked in buffer containing detergents while the detergent-free enzyme still had significant amounts of high molecular weight species when cross-linked in the same buffer. These results prove that rLp-PLA2 does quickly self-associate and form polymers upon dilution in the absence of lipid substrates or detergents. The detergents do not reduce the reactivity of EGS in the cross-linking of rLp-PLA2 because the control experiments to internally cross-link IgG by EGS were not altered by the presence of the same detergents (data not shown).

In the characterization of rLp-PLA2 by size exclusion chromatography, it was found that the recovery yield in the absence of detergents was very low as shown by the CAM activity of the collected fractions (FIG. 1). By including 10 mM CHAPS in the chromatography buffers, not only the recovery yield was improved but also the molecular size of the rLp-PLA2 was reduced. This suggests that the enzyme may not exist as the monomeric form in the absence of detergents. Indeed, fractionation of the C-terminal His-tag rLp-PLA2 in the absence of detergents and assaying the fractions by HisGrap-ELISA using rabbit anti-Lp-PLA2 polyclonal antibodies, which detects both the native and denatured rLp-PLA2, we demonstrate that a lower molecular weight mass peak without enzymatic activity was missed by the CAM assay (FIGS. 1A-1C). It is unlikely that the mass without activity comes from the impurity that cross reacts with the polyclonal antibodies because the recombinant protein has been purified to highly homogeneous purity and subjected to SDS-PAGE and Western Blotting analyses (data not shown). The results suggest that it is the monomeric rLp-PLA2 that may not be stable in the absence of detergent and it can also be inferred that the enzyme may form oligomers in the absence of detergents in order to keep the hydrophobic sites away from aqueous solvent. When diluted, there will be more monomeric rLp-PLA2 formed because of the increase in the rate for dissociation and the decrease in the rate for oligomer formation. Thus, dilution will cause more inactivation of the enzyme in the absence of detergents. This is indeed the case observed in our study. To explain the dependence of the inactivation rate on the rLp-PLA₂ concentrations, one model is that oligomization and dissociation are reversible steps but the denaturation is an irreversible step. At high protein concentration, the rate of oligomer formation is fast and the monomeric rLp-PLA2 is less abundant and, therefore, the enzyme is stable. Dilution or breaking protein-protein interaction will increase monomeric rLp-PLA2 and, in the absence of substrate or detergents, it will result in the inactivation of the enzyme. It is possible that the monomeric rLp-PLA2 has widely open hydrophobic regions, such as the interfacial or substrate-binding site, as illustrated by the crystal structure. Access of these hydrophobic regions by aqueous solvents would result in the disruption of the rLp-PLA2 molecular structure and, thus, the enzyme would block these hydrophobic regions by self-association or oligomerization to keep the water molecules away in the absence of other hydrophobic entities. In the presence of other hydrophobic particles such as detergent or substrate micelles, or lipid particles, the rLp-PLA2 molecule would form complexes with these compounds to cover its hydrophobic regions. Unlike enzymes such as Rhizomucor miehei lipase, which has a “lid” to cover its active site in the absence of substrate, Lp-PLA2 probably has to form complex to shield aqueous solvent from the empty hydrophobic substrate binding site. If a proper complex partner is not available, the monomeric Lp-PLA2 will form a self-complex and it does not stop as a dimer but can extend to oligomers with different unit length. By associating together, Lp-PLA2 molecules reduce the hydrophobic surface area exposed to water and minimize the disruptive effect. The deuteration experiments by using DXMS method have shown that the active site residues of Lp-PLA2 barely exchange with solvent. This does indicate that the active site of Lp-PLA2 is in a closed form. Self-oligomization was previously reported for a Group VI Ca²⁺-independent cytosolic phospholipase A2 although the functional benefit for the enzyme was not discussed. Self-oligomerization is not uncommon for biological active proteins. One of the best studied examples is insulin. In most of the cases, protein self-association plays important roles in protein biosynthesis and preservation of protein functional activities. In other cases, protein self-association is to form specific structures such as apolipoprotein A (ApoA). In summary, the results shown here demonstrate that if Lp-PLA2 monomers fail to form a complex or oligomer during dilution into low concentration, in the absence of lipid substrates or detergents, the enzyme will go through irreversible denaturation possibly initiated by the disruption of hydrophobic regions in the aqueous milieu. In the presence of detergents, Lp-PLA2 will associate with detergent micelles and stabilize as a monomer. The roles of lipid particles such as LDL or HDL in human plasma may be just like detergent micelles in these experiments; the lipid particles act as the chaperones to stabilize the Lp-PLA2 in circulation possibly by binding to its hydrophobic interface. Reducing lipids by statins or fibrates was found also reduced Lp-PLA₂ mass and activity.

In addition to finding that recombinant Lp-PLA2 may be effectively stabilized by include micelles in the buffer solution to protect the rLp-PLA2, additional modifications to stabilize a calibration solution of rLp-PLA2, including salt content, additional detergent, pH, and the like have been examined to determine how to prepare stable calibration solutions with a long shelf-life.

A series of four real-time stability studies were performed to identify stability factors for an Lp-PLA2 assay, and particularly the recombinant Lp-PLA2 calibrators used for the assays. In many of these examples, the Lp-PLA2 assays are mass (e.g., immune-) assays. In these example, the Lp-PLA2 assay may be referred to as a “PLAC ELISA kit” and the associated calibration standard(s).

In the examples described below, a combination of component swapping assays and designed experiments were utilized to characterize raw materials and their possible (e.g., desired or optimal) concentrations. One goal was to utilize robust design principles to maximize stability and minimize variation of the calibrator formulation in order to provide excellent product performance and desired expiration dating of a kit. It should be understood that while these studies provide actual experimental results, other factors may also be taken into account in designing a calibration standard. For example, other factors may additionally affect the stability or other parameters of interest in a calibration standard (e.g. color, turbidity, viscosity, etc.) and a calibration standard may be contemplated that takes, on the whole, multiple factors into consideration. An individual component may (or may not) be used under its optimal performance. The result may be that a component or formulation described herein may be useful at a different concentration or in combination with other factors than what the data, on face value, may suggest. Additionally, any parameter or component referred to or described herein is recognized as one that may be contemplated for generating a calibrator formulation regardless of a specific experimental result. In particular, as some unpredictability exists with any experimental system (no matter how well designed or executed), an individual experimental result should not be taken as the only possible outcome.

Thus, the calibrator solutions and assays and kits including them are not limited to use with one particular type of assay (e.g., a “PLAC test ELISA assay). Other assays have been examined for calibrator performance as described herein, including a “Auto-CAM” enzymatic activity assay for Lp-PLA2. Thus, the same calibrators may be used in other platforms for the analyte Lp-PLA2 involving clinical analyzers, and these calibrator results may be applicable to the other platforms; calibrators may be corrected for differences in assay temperatures, ionic strengths of reagent systems, identities of the detergents used (and their corresponding critical micelle concentrations), different length assay times and intrinsic on-rates/off-rates for a given antibody:antigen equilibrium binding state (or, more likely, its non-equilibrium binding state) at any given set of assay conditions, etc.

The first stability study (Example 1, below) described is a systematic comparison of a panel of detergents in the context of an existing calibrator diluent formulation. The effect on calibrator stability of substituting a battery of detergents into an existing calibrator formulation at different concentrations was assessed. This study included substituting various CHAPS analogues as well as CHAPS supplied from various vendors/grades/lot numbers for the primary detergent into an existing calibrator formulation. The results of this study identified performance differences between CHAPS lots as well as concentration-dependent effects of CHAPS on calibrator stability. More generally, the results strongly suggest that detergent micelle formation may be an important factor for Lp-PLA2 protein stability in some formulations.

The second stability study (Example 2, below) was designed to explore the effect of calibrator diluent raw material quality on calibrator long-term stability. Thirty-six separate raw material combinations sourced from at least two different vendors or grades were compared. In this study, the raw materials tested included CHAPS, BSA, DTT, sodium chloride, water, glycerol and ProClin-300 (present/absent). The results confirmed that the CHAPS detergent is an important factor for calibrator stability in some cases. In addition, different combinations of CHAPS and BSA interacted synergistically to affect both stability and precision. This study also provided evidence that choice of glycerol (including grade of glycerol) can affect calibrator stability.

The third stability study (Example 3, below) was a response surface design experiment that explored the effects on calibrator long-term stability of experimentally manipulating Tris buffer pH, Tris buffer concentration, CHAPS concentration and DTT concentration. First, the experimental results indicated that incrementally increasing the CHAPS concentration above a standard concentration has a positive effect on calibrator stability. Conversely, decreasing the CHAPS concentration below a standard concentration has an adverse effect on calibrator stability. Second, the labile reducing agent DTT was identified as a useful effector of calibrator stability in some cases.

The fourth stability study (Example 4, below) explored the effects of differences in Tris buffer composition, Tris buffer pH and different grades and/or lots of Probumin BSA. In the context of the calibrator formulation, minor perturbations in buffer composition seemingly had no discernable effect on calibrator stability. The effects of surveying different grades of BSA on calibrator stability suggested idiosyncratic differences that vary by the lot number of BSA used rather than any systematic differences based on the grade of BSA used. Interestingly, a process change in the starting and final pH of the calibrator matrix conferred enhanced precision in this study.

Taken together, the results of these studies suggest that raw materials in calibrator diluent may include CHAPS detergent at appropriate levels, a reducing agent (DTT), and glycerol. A combination of additional incoming quality control specifications/testing (CHAPS purity, glycerol quality), manufacturing process controls (ensuring DTT integrity) and additional critical raw material validations studies (increased CHAPS concentration, pH adjustment) may be useful. In these studies, the percent stability and precision of the calibrator formulations were used as a more direct response rather than the indirect response of serum percent stability. In addition, the precision of the calibrators is much better than the precision of serum samples, particularly those serums with high Lp-PLA2 analyte levels. Utilizing the stability and precision of the calibrator formulations as direct responses should allow both more sensitivity in the measurement of the responses as well as a general reduction in the signal:noise ratio compared to assaying the serum samples as a response.

In the examples below, the following terms may be used and understood as follows:

Coefficient of Variation (% CV) may refer to a measure of the relative variation of distribution independent of the units of measurement; the standard deviation divided by the mean, expressed as a percentage.

Critical Micelle Concentration (CMC) may refer to the concentration of surfactants above which micelles form and almost all additional surfactants added to the system go to micelles

Designed Experiment (DOE) may refer to experimental methods used to quantify measurements of factors and interactions between factors statistically through observance of forced changes made methodically as directed by mathematically systematic tables.

Full Factorial Design may refer to A DOE that measures the response of every possible combination of factors. These responses are analyzed to provide information about every main effect and every interaction effect. The approach used in screening experiment to identify main effectors and to identify first-degree polynomial effects.

Gauge Analysis may refer to attribute gauge analysis that gives measures of agreement across responses in graphs summarized by one or more X grouping variables.

Response Limit may refer to the specification of one of the possible goals for a DOE response variable, such as percent stability. JMP allows one to choose from the following goals: Maximize, Match Target, Minimize, or None.

Response Surface Design may refer to a type of DOE experiment that allows the interactions between factors to be mapped and it identifies quadratic (second degree polynomial) effects. It is typically used to optimize a process and/or make it more robust.

Robust design may refer to the practice of making the response of a system insensitive (or robust) to uncontrollable variation by desensitizing the product to these potential sources of variation.

Variability Chart may refer to a variability chart that shows how a measurement varies across categories. The mean, range, and standard deviation of the data can be analyzed in each category. The analysis options assume that the primary interest is how the mean and variance change across the categories.

Variance Inflation Factor (VIF) may refer to a large VIF value indicates that the X variable is highly correlated with any number of other X variables. VIF values between 1-3 are no problem; VIF's between 4-7, are problematic and should be removed; VIF's >8, must be removed.

### Example 1

### Detergent Comparison Study

Purpose: To compare the effect of substituting various alternative detergents, different CHAPS analogues and CHAPS raw materials sourced from different vendors/grades in a short-term stability study.

Materials:

PLAC ELISA kit, P/N 90123, L/N 1001003

Antigen: P/N 26203, L/N 1010057

BSA: Roche Diagnostics, P/N 03117405001, L/N 70189921

Glycerol: EMD, P/N GX0185-5, L/N 41116133

Detergent Screening Kit (all Dojindo), P/N DS06, L/N CT717: CHAPS (CAS #75621-03-3), L/N CM607; n-Dodecyl-β-D-maltoside (CAS #69227-93-6); n-Octyl-β-D-glucoside (CAS #29836-26-8); Sodium cholate, monohydrate (CAS #73163-53-8); MEGA-8 (CAS #85316-98-9)

Modified CHAPS analogues (all Dojindo): BIGCHAP (P/N D043, L/N CT710; CAS #86303-22-2); CHAPSO (P/N C020, L/N CT711; CAS #82473-24-3); deoxy-BIGCHAP (P/N D045, L/N CT712; CAS #86303-23-3);

CHAPS from standard vendor (all Sigma): CHAPS: P/N C3023, L/N 018K53003 (lot #1); CHAPS: P/N C3023, L/N 040M5319V (lot #2); CHAPS, BioXtra: P/N C5070, L/N 18K530041V (lot #3)

Experimental Procedures:

Experimental Plan: The eleven different detergent variants in this study included each of those included in Dojindo “First Choice” detergent screening kit (CHAPS, n-Dodecyl-β-D-maltoside, n-Octyl-β-D-glucoside, sodium cholate and MEGA-8), various CHAPS analogues (Dojindo detergents CHAPSO, BIGCHAP, deoxy-BIGCHAP), and various grades/lot numbers of Sigma CHAPS (including two lots from a current grade of CHAPS). All the detergents were substituted into a standard calibrator diluent formulation at four concentrations each in a linear titration series. The concentration range surveyed for each detergent was based on each individual detergent's published critical micelle concentration (CMC). Most detergents were also tested at one concentration above the CMC and two concentrations below the CMC, with the single exception being the MEGA-8 detergent. The MEGA-8 detergent presented a technical challenge with respect to testing above its published CMC (58 mM). With this consideration in mind, the highest concentration of MEGA-8 detergent surveyed was 50 mM. In total, forty-four different calibrator diluent formulations spiked with antigen at a single analyte concentration were tested in a short-term refrigerated stability study.

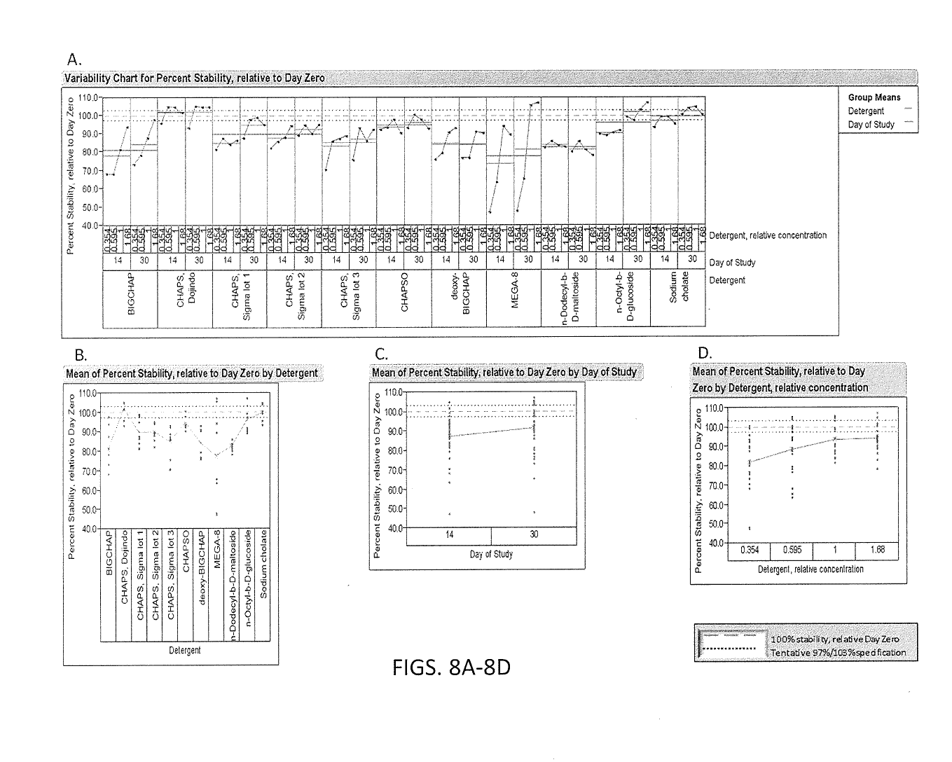

Experimental Details: The reactions were systematically assembled as two master-mixes to facilitate the highest detergent concentrations going into solution quickly into the standard calibration diluent formulation. This is due to the formulation's high ionic strength, contributed principally by the 2.857 M NaCl. Mastermix A (5×) was a pre-formulated, buffered isotonic salt solution into which BSA and Proclin-300 dissolved until a homogeneous solution is achieved. Master-mix B (1.33×) was a pre-formulated, buffered high salt solution with the reducing agent DTT dissolved until a homogeneous solution is achieved. An appropriate volume of each of the two master-mixes is added to each of the forty-four formulations with an appropriate volume of a concentrated detergent stock solution and HPLC-grade water (if necessary) to achieve the desired final detergent concentrations for the forty-four variants of the calibrator diluent formulation as indicated in the legend for FIG. 8A-8D. An appropriate amount of 100% glycerol is added so that the final concentration of all non-detergent raw materials is at their standard calibrator diluent concentrations per MP-21090. After mixing, the recombinant protein Lp-PLA2 was added to each formulation in order to achieve an intended final analyte concentration of approximately 250 ng/mL. The calibrator formulations were put on stability at refrigerated temperature. Three stability timepoints are taken at Day 0, Day 14 and Day 30 using a PLAC ELISA kit.

Results

A. Detergent Comparison Study, One Month

Eight different detergents were compared in this one month study, including four individual lots of CHAPS sourced from two different vendors, for a total of eleven variations (FIGS. 8A-8D). Each detergent/vendor/grade/lot variation was assayed for calibrator stability at four concentrations, with most of the detergents being tested using concentrations that bracket their published Critical Micelle Concentration (CMC). The one exception was the MEGA-8 detergent due to practical considerations of its extremely high CMC value (See legend to FIGS. 8A-8D). In addition to assaying at CMC, two concentrations lower than the published CMC and one concentration higher than the published CMC were assayed. A linear concentration titration with a dilution factor of 1.68-fold was tested for each detergent in order to specifically accommodate the fold-concentration difference between the standard CHAPS concentration used in the calibrator diluent formulation (e.g., 4.76 mM) and its published CMC (e.g., 8.00 mM). Published studies indicate that micelle formation by CHAPS is concentration-dependent High salt conditions, such as those found in the calibrator diluent formulation, favor micelle formation. In contrast, lowering the temperature, a situation that occurs upon initiating a real-time stability study, disfavors micelle formation. In this short-term refrigerated stability study, timepoints were taken at both 14 and 30 days and then compared on a percentage basis to the Lp-PLA2 analyte value of the initial time point.

A comparison of the percent recovery of the each of the forty-four calibrator formulations at the Day 14 and Day 30 timepoints is shown in FIG. 8A. Percent stability was calculated based on analyte values established using refrigerated finished good calibrators. The provisional 97%-103% calibrator stability specification is shown by the red hatched lines with the target stability of 100% relative to Day 0 shown as a green dashed line (FIG. 8A-8D). The mean analyte concentration in the CHAPS-formulated calibrators including all lots and all three timepoints was 418 ng/mL. The CHAPS (from Dojindo) and sodium cholate were among the best performing detergents with regard to short-term refrigerated stability when both subsequent timepoints are compared to their individual Day 0 analyte value (FIG. 8A). Across the four concentrations and two timepoints surveyed, the gauge analysis indicates that the mean percent stability for these two detergents tested even meets the provisional 97%-103% stability specification for percent recovery at individual timepoints (FIG. 8B). The detergent, n-dodecyl-β-D-maltoside, was the worst performer of the group tested with even the highest detergent concentrations not resulting in good Lp-PLA2 stability relative to Day 0 (FIG. 8A). On average, the analyte values of the forty-four calibrators trended incrementally higher on Day 30 than on Day 14 (FIG. 8C). When the percent stability data is trended as function of detergent concentration, the detergents' collectively show an interesting profile: In general, the percent stability drops off quickly once the detergent concentration drops below its individual CMC (FIG. 8D), strongly suggesting micelle formation may be necessary for optimal Lp-PLA2 stability.

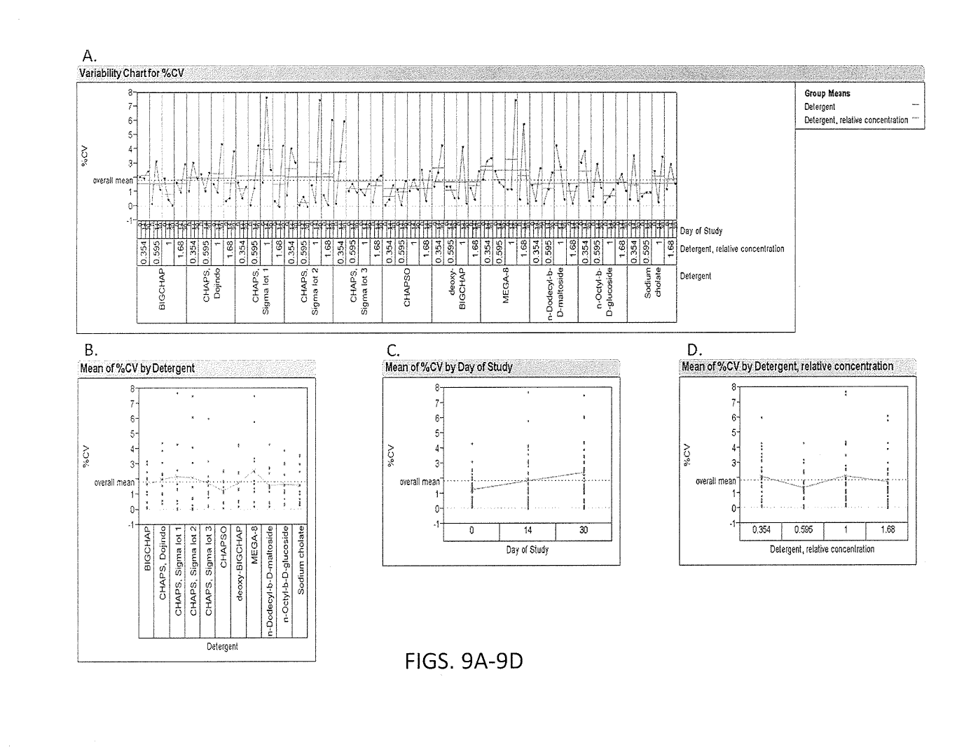

A comparison of the imprecision (e.g., by coefficient of variation [% CV], n=2 replicates per formulation condition per time point) of the each of the forty-four calibrator formulations at each of the three timepoints is shown in FIG. 9A. The overall mean % CV for the forty-four detergent conditions across all timepoints was 1.81%. Of the eleven formulation surveyed, the gauge analysis indicates that the CHAPSO detergent demonstrates the best precision whereas the MEGA-8 demonstrated the worst precision (FIG. 9B). Of the four CHAPS lots tested from two vendors, small differences in imprecision can be detected. The Sigma lot #3 (BioXtra grade; P/N C5070, L/N 18K530041V) demonstrated the best precision, and the single lot of Dojindo CHAPS demonstrated the second-best precision (FIG. 9C). The other two lots of Sigma CHAPS (P/N C3023; lot #1, L/N 018K53003; lot #2, L/N 040M5319V) were a manufacturing grade used for calibrator diluent production and both showed worse mean precision than the grand mean of 1.82% (i.e., for all detergents tested; FIG. 9B). Across all the detergents surveyed in this study, there was no obvious relationship between detergent concentration and precision (FIG. 9C). There was a discernable trend over the forty-four formulations of the precision getting gradually worse (i.e., higher % CV's) over the course this short-term stability study of only thirty days (FIG. 9D).

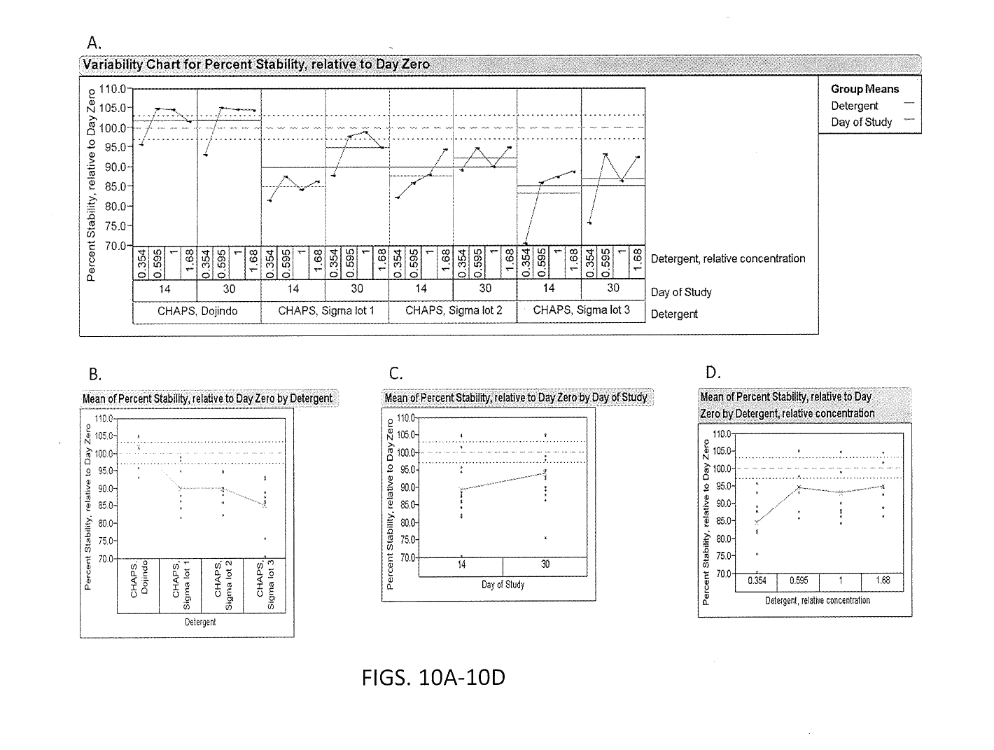

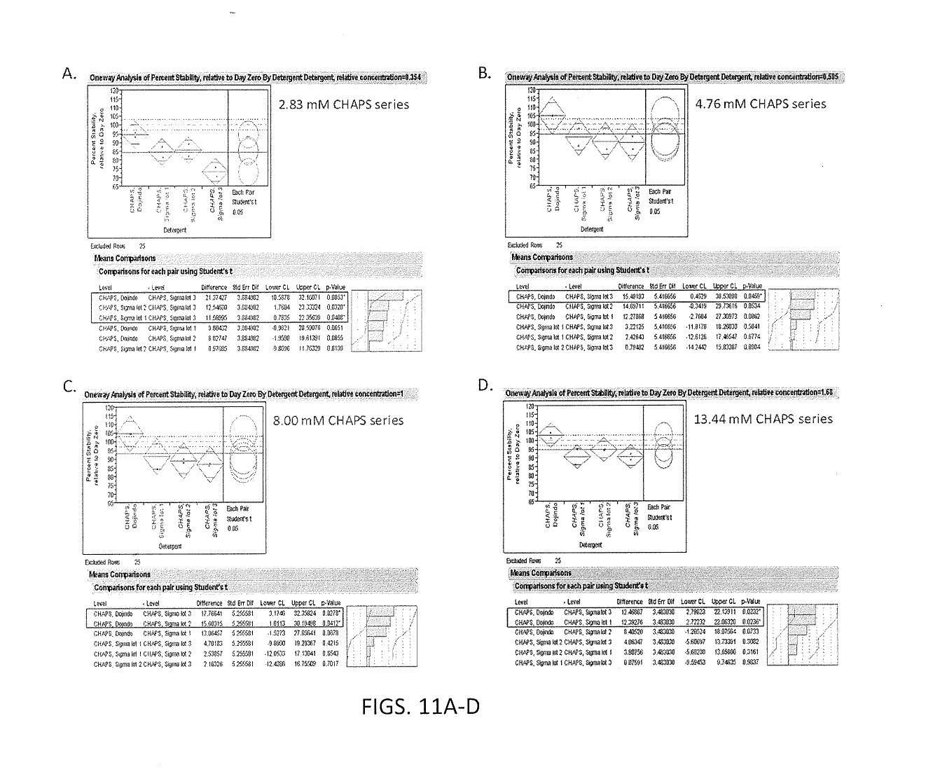

The percent stability data for the sixteen CHAPS-based calibrator formulations (four lots at four concentrations each) were parsed out from the stability data from other detergents and analyzed in more detail in FIGS. 10A-10D and FIGS. 11A-11D. In general, the lot of CHAPS from Dojindo (P/N C008, L/N CM607) showed superior stability compared to all three lots of Sigma CHAPS at both the Day 14 and Day 30 timepoints (FIG. 10A). The worst performer was the Sigma lot #3 (BioXtra; P/N C5070, L/N 18K530041V). Across the four concentrations and two timepoints surveyed, the gauge analysis indicates an overall trend that the lot of CHAPS from Dojindo showed superior stability compared to all three lots of Sigma CHAPS tested in this study (FIG. 10A). Similar to the analysis with the entire battery of detergent formulations (FIG. 10C), the percent recovery of the sixteen CHAPS-formulated calibrators trended incrementally higher on Day 30 than on Day 14 (FIG. 10C), possibly reflecting some effect related to day-to-day variability. When the percent stability data for the CHAPS-formulated calibrators alone is trended as a function of detergent concentration, the trending indicates ˜10% drop in percent stability when the CHAPS concentration drops below a concentration (i.e., 4.76 mM, indicated in FIG. 10D as the coefficient 0.595 [of CHAPS CMC value]). Above the standard concentration of CHAPS, the percent stability of the calibrators plateaus (FIG. 10D), at least in the context of this short-term stability study of 30 days duration.

A Student's t-test was performed to compare the mean percent stability of the two timepoints (i.e., the mean of the percent stability Day 14 and Day 30 timepoints at each CHAPS concentration) for statistically significant differences (p value <0.0500; FIG. 11A-11D). The Student's t-test was the comparison selected because the primary goal was to detect if there is a difference in the mean value for each of the individual Sigma lots compared to the single lot from Dojindo. A secondary goal was to the Student's t-test compare the means between individual lots of CHAPS from Sigma. The Dojindo lot of CHAPS showed statistically significant differences in the mean percent stability relative to Sigma lot #3 at detergent concentration tested (FIG. 11A-11D). In fact, the Sigma lot #3 shows statistically significant differences with all of the other lots of CHAPS at the lowest detergent concentration (FIG. 11A). The Dojindo shows statistically significant differences at some detergent concentrations with Sigma lot #1 (13.44 mM; FIG. 11D) and Sigma lot #2 (8.00 mM; FIG. 11C). At other detergent concentrations, the differences in the means in the percent stabilities just miss the criteria for statistical significance in the comparison between the Dojindo CHAPS and the Sigma lot #1 (e.g., p-values of 0.0651, 0.0862 and 0.0678 in FIGS. 11A, 11C and 11D, respectively) and the Sigma lot #2 (e.g., p-values of 0.0855, 0.0862 and 0.0678 in FIGS. 11A, 11B and 11C, respectively). Minimally, these results suggest that there are differences in calibrator stability performance between the different lots of CHAPS tested here. These differences in stability can be seen as early as fourteen days post-formulation.

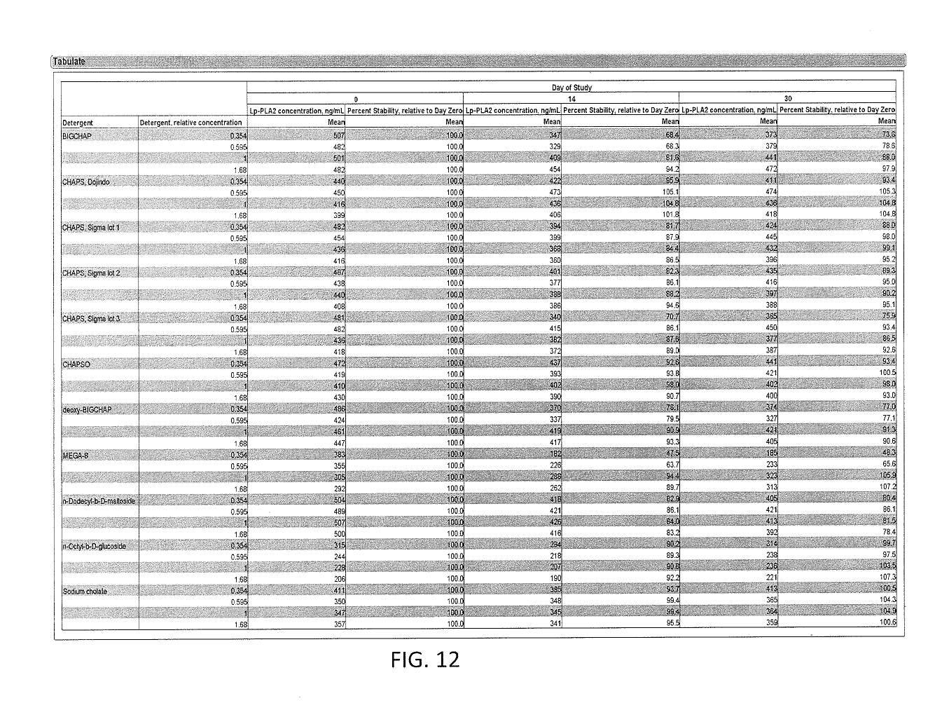

The experimental results from the Detergent Comparison study are summarized in FIG. 12. Even at the Day 0 time point, not all of the detergents surveyed give a similar range of Lp-PLA2 analyte values (in ng/mL) even within the titration series. In fact, some of the detergents give radically different analyte values across the entire titration series relative to CHAPS titration series with the biggest outlier being the detergent n-octyl-β-D-glucoside (FIG. 12). In contrast, the various lots of CHAPS-formulated calibrators yield very similar analyte values at a given detergent concentration. It should be noted, though, that Lp-PLA2 analyte values, in general, incrementally decrease as a function of increasing CHAPS concentration. Collectively, these results suggest the following two implications: (1), the final concentration of the CHAPS detergent in the calibrator diluent formulation ultimately has a subtle effect on the exact Lp-PLA2 analyte values obtained, and, (2), switching to an alternate detergent may have a profound effect on the exact Lp-PLA2 analyte values obtained.

### Example 2

### Material Variation Study

Purpose: To explore the effect on calibrator stability and precision of substituting different vendors' and/or different grades/lots of the various calibrator raw materials into the context of two different collections of the remaining raw materials.

Materials:

PLAC ELISA kit: P/N 90123, L/N 1012045;

Antigen: P/N 26203, L/N 1010057;

Tris base;

Standard Grade: Sigma P/N 1503, L/N 040M5439V: Test Grade: Research Organics P/N 3094T, L/N A82450;

CHAPS: Standard Grade: Sigma C3023; Lot #1: L/N 018K53003; Lot #2: L/N 040M5319V; Lot #3: L/N 077K530012; Lot #4: L/N 100M53082V. Test Grade: Dojindo C008; Lot #5: L/N CY783; Lot #6: L/N CY784; Lot #7: L/N CY785.

BSA: Standard Grade: Millipore “Universal”, P/N 81003; Lot A: L/N 692 (current Production lot); Lot B: L/N 693; Lot C: L/N 694. Test Grade: Millipore “Diagnostic”, P/N 82045; Lot D: L/N 452; Lot E: L/N 454; Lot F: L/N 453.

DTT: Standard Grade: Sigma P/N D0632, L/N 031M1753V. Test Grade: BioVectra P/N 1370, L/N 37383;

NaCl: Standard Grade: Sigma P/N 59888, L/N 040M02279V; Test Grade: Research Organics P/N 0926S, L/N Z80586;

Water: Standard Grade: JT Baker P/N 4218, L/N J50E00; Test Grade: Ricca P/N 91901, L/N 1103234

Glycerol: Standard Grade: EMD P/N GX0856, L/N 50242049; Test Grade: Research Organics P/N 9580G, L/N B90864

ProClin 300: Standard Grade: Supelco P/N 48914, L/N LB82798; Test Condition: Absent from formulation

Hydrochloric acid, Mallinckrodt, P/N 2062-46, L/N 149028;

Millipore Steriflip Express Plus, P/N SCGP00525, L/N MPSF006562;

Experimental Procedures:

Experimental Plan: Two calibrator formulation groups of raw materials were established: one with a standard group of manufacturing raw materials and one with a test group candidate raw materials of a different grade and/or from a different vendor. The test group consisted of raw materials sourced either from potential alternate vendors of raw materials that had showed promise in earlier experiments (e.g., Dojindo CHAPS) or from vendors of raw materials with claims of exceptionally high purity (e.g., ultra-pure grade or diagnostic grade). Individual raw materials were systematically tested in each of two contexts: the standard group of raw materials and the test group of candidate raw materials. In addition to the dry raw materials obtained as salts or powders, two grades of water were surveyed in this comparison study (HPLC grade and USP, Ph. Eur. grade). In some cases, certain candidate critical raw materials (CHAPS and the BSA) were evaluated by screening multiple lots of each grade of raw materials against both raw material groups. The one exception to the alternate sourcing of the raw materials was the Proclin-300. As there is only one vendor, the variable tested was the presence/absence of this preservative in the context of both the standard and test groups of the other raw materials. In total, thirty-six different calibrator diluent formulations reflecting a single substitution of each raw material were created. Each formulation was spiked with antigen at a single analyte concentration were tested in a long-term refrigerated stability study.

Experimental Details. Concentrated stock solutions were created for the two groups of the following raw materials: iris base (1.00 M), CHAPS (0.050 M), DTT (1.00M), sodium chloride (5.00M). All the raw materials in the standard group were formulated using the Ricca USP grade water, and all the raw materials in the test group were formulated using the JTBaker HPLC grade water. Proclin300 was added neat, as needed. For purely technical reasons, the appropriate grade/lot of BSA was added as powder to each formulation. Thirty-six reactions were systematically assembled as two separate master-mixes from the raw material stocks ((5× and 1.25×, analogous the formulation work described)). An appropriate volume of the designated grade of undiluted glycerol was added to each of the thirty-six formulations. After mixing, each formulation was filtered using a 50 mL Millipore Express Plus filtration unit followed by the addition of the recombinant protein Lp-PLA2 to a final analyte concentration of approximately 250 ng/mL, as described. The calibrator formulations were put on stability at refrigerated temperature. The FG calibrators were split into two sets, one stored at refrigeration temperature and one stored frozen at −70° Celsius. Stability timepoints were taken on Day 0, Week 1, Week 2, Week 4, Week 6, Week 8, Month 4, Month 5, Month 6, Month 7, and Month 9 using the PLAC ELISA kit. Specific activity of the DTT was calculated using a quantitative sulfhydryl assay with free cysteine as a standard and following the manufacturer's recommended instructions (Pierce kit #22582; see LNB 0555-108 to 0555-113).

Results

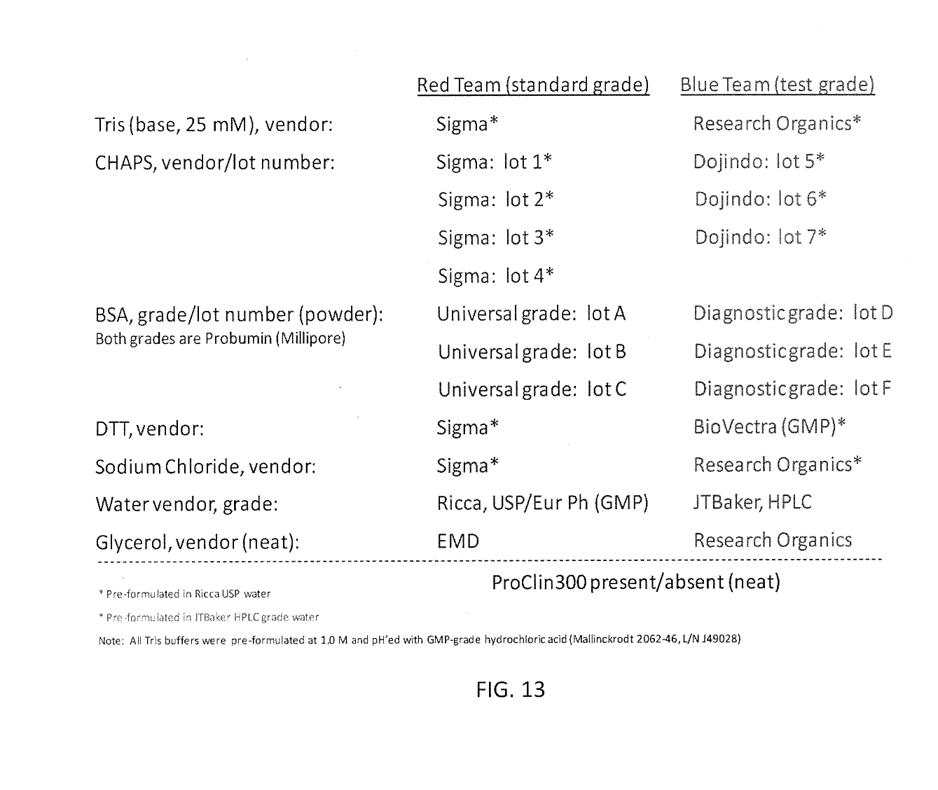

This long-term stability study is raw material component swapping study in which each raw material used in the calibrator diluent formulation is sourced from two different vendors and/or from two different reagent grades (with the exception of the Proclin-300 preservative, in which the variable is presence/absence). In many cases, the raw materials are of two different grades (FIG. 13), with one being a standard grade (Red Team) and, typically, a test grade (Blue Team) being a high-purity competing raw material. The “Red Team” of raw materials used all the standard grade of raw materials with the exception of the water. A calibrator diluent used a pharmaceutical grade of water known as USP grade. A USP/Eur. Ph.-certified, GMP grade of water was sourced from Ricca for use as a raw material in the standard grade combination. In 2011, a grade of water known as HPLC grade was sourced from JT Baker (MSS-10107) and replaced house de-ionized water used in manufacturing starting with ELISA kit lot number 1012111. This HPLC grade of water was used as part of the test grade of raw materials. In addition to the water grade comparison, the Tris buffer, CHAPS, DTT, sodium chloride and glycerol were sourced from two different vendors. The BSA was sourced from the standard vendor, but two different grades were compared, Universal grade and Diagnostic grade. In addition, analytical testing was performed on the specific activity of each grade of DTT after formulation into calibrators to show equivalence. The mean specific activity, relative to the free cysteine standard curve, for each vendor's DTT was calculated on Day One, post-formulation (Sigma DTT: mean [+/−STDEV]=0.404+/−0.108 mM; BioVectra, mean [+/−STDEV]=0.460+/−0.047 mM; n=18 formulations each vendor's DTT). In the case of the CHAPS and BSA, at least three lots of each grade were included in the study. The individual lots of CHAPS used in the standard formulation and test formulation were Sigma lot 1 (lot #018K53003) and Dojindo lot 7 (L/N CY785), respectively. The individual lots of BSA used in the standard formulation and test formulation were Universal grade lot A (lot #692, a production lot) and Diagnostic grade lot F, respectively. The experimental design consisted of systematically substituting individual raw materials from a particular grade/lot from one set of raw materials into the other collection of raw materials.

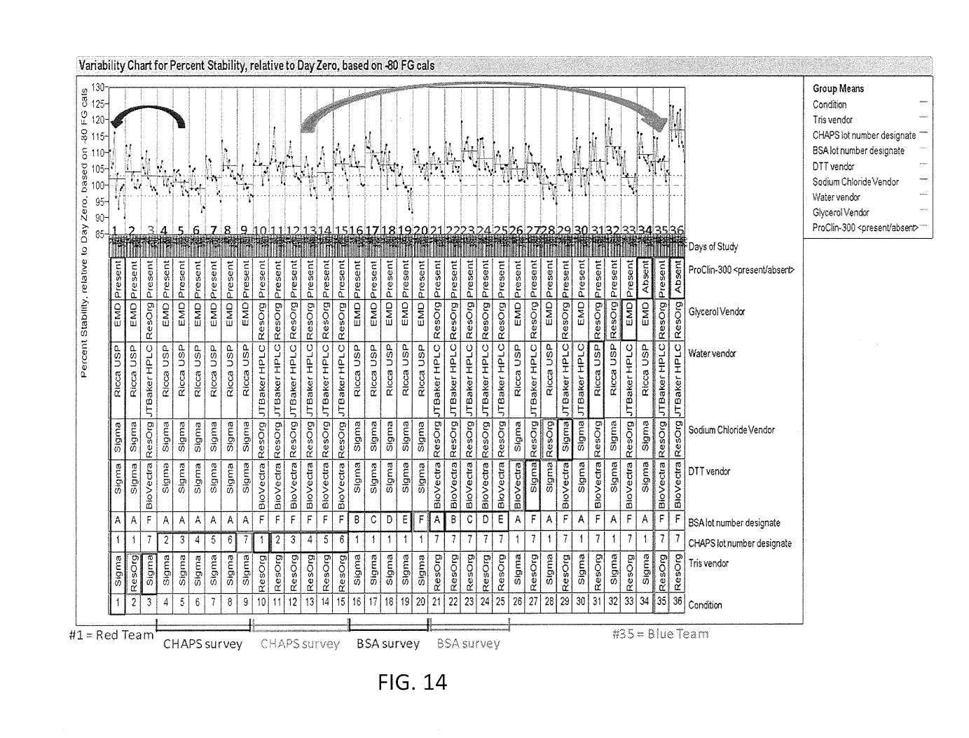

The percent stability for each of the thirty-six calibrator diluent formulations for first nine months of timepoints is shown in FIG. 14. Percent stability was calculated based on analyte values established using finished good calibrators that were frozen prior to initiating the study. The provisional 97%-103% calibrator stability specification is shown by the red hatched lines with the target stability of 100% relative to Day 0 shown as a green dashed line (FIG. 14). The “Red Team” of raw materials is Condition #1 and the Blue team of raw materials is Condition #35. Representative examples of the substitution of individual grades/lots are shown in Conditions #2 and #3: The Tris buffer from the vendor Research Organics is substituted into the context of the Red Team's remaining raw materials in Condition #2, and the Tris buffer from the vendor Sigma is substituted into the context of the Blue Team's other raw materials in Condition #3 (FIG. 14). Different lots of CHAPS raw materials are substituted into the Red Team and Blue Teams remaining raw materials in Conditions #4-9 and Conditions #10-15, respectively. Different grades/lots of BSA raw materials are substituted into the Red Team and Blue Teams remaining raw materials in Conditions #16-20 and Conditions #21-25, respectively. Similarly, substitution of different lots of DTT, sodium chloride, water, glycerol and ProClin300 (presence/absence) are shown for the remaining conditions (i.e., Conditions #26-34, #36)

The best performing formulation of these thirty-six calibrator conditions is Condition #5 with respect to achieving the provisional 97%-103% calibrator stability specification for the majority of the eleven timepoints (Compare Condition #5 to Condition #1, Condition #4, and Conditions #6-9; FIG. 14). Condition #5 is part of the battery of CHAPS lot substitutions formulated in the context of the Red Team's raw materials. Notably, the calibrator formulation Condition #5 has the substitution of CHAPS lot #3 (Sigma C3023, L/N 077K530012) for CHAPS lot #1 (Sigma C3023, L/N 018K53003) in the calibrator diluent.

Other well-performing formulations of these thirty-six calibrator conditions were Condition #4, Condition #9 and Condition #28. Condition #4 and Condition #9 are part of the series of CHAPS raw material survey, substituting CHAPS lot #2 (Sigma C3023, L/N 040M5319V) and CHAPS lot #7 (Dojindo C008, L/N CY785), respectively, for CHAPS lot #1 (i.e., Sigma C3023, L/N 018K53003 found in Condition #1; FIG. 14). These experimental results indicate that the substitution of one lot of CHAPS for another lot of CHAPS in an otherwise identical formulation can result in a dramatic difference in long-term stability performance. Taken together, these results strongly suggest that the choice of CHAPS lot is a critical raw material in the calibrator diluent formulation in some cases.

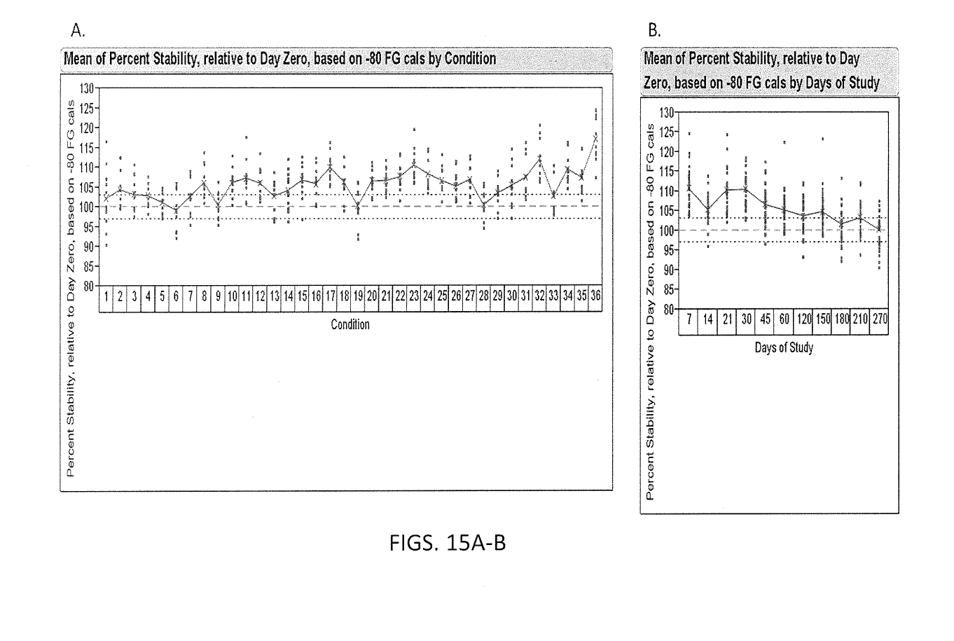

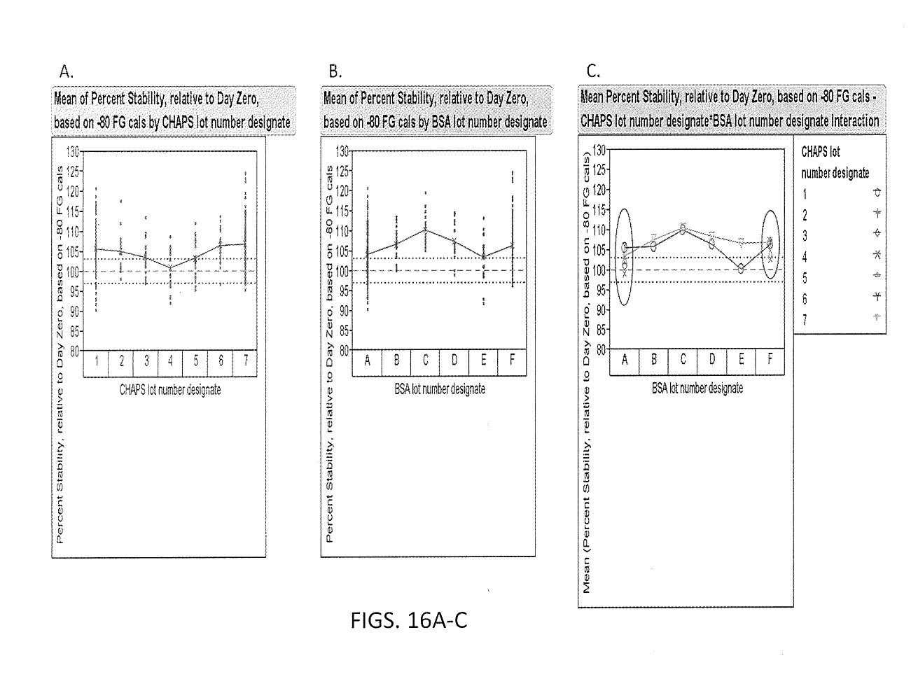

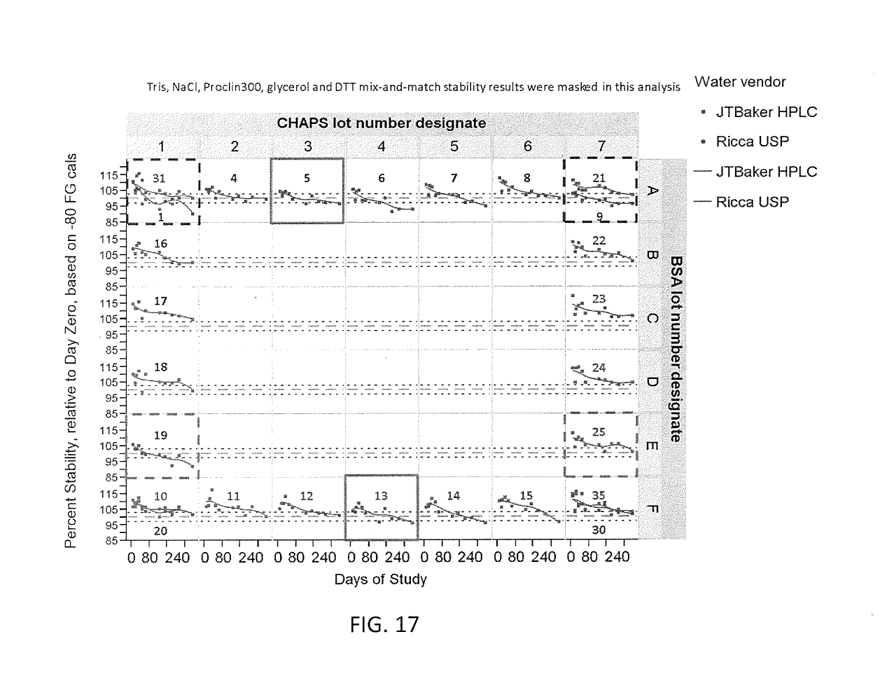

A gauge analysis shows that the overall trending of the long-term stability results as a function of calibrator condition (FIG. 15A). In addition to showing the anticipated decrease in calibrator stability over time (FIG. 15B), the gauge analysis also showed the trending of long-term stability as a function of the various raw material used (FIGS. 16A-16C and 17). Among the Red Team collection of raw materials, the previously-mentioned Conditions #4, #5, #9 and #28 are among the best performers with a majority of their stability timepoints falling within the 97%-103%% specification. Among the Blue Team collection of raw materials, Condition #13 is the best formulation condition for achieving the 97-103% specification (e.g., compare Condition #13 to Condition #35; FIG. 14 and FIG. 15A). The best performer, Condition #13, is part of the CHAPS substitution series for the Blue Team's collection of raw materials, and it substitutes CHAPS lot #4 (Sigma, C3023, L/N 100M53082V) for CHAPS lot (Dojindo C008, L/N CY785). In general, the stability follows a very similar trending for the CHAPS lots across both collections of raw materials (compare Conditions #4, #5, #6, #7 and #8 to Conditions #11, #12, #13, #14, #15, respectively; FIG. 14). The gauge analysis indicates that CHAPS lot #4 yielded the optimal results with respect to long-term stability trending across the entire experiment (FIG. 16A).

Two different grades of bovine serum albumin (BSA), the standard production “Universal Grade” and the “Diagnostic Grade”, from Millipore were also surveyed in this raw material comparison study (three lots each; compare Conditions #1, #16-20 in the context of the Red Team to Conditions #21-25 and Condition #35 in the context of the Blue Team, respectively; FIG. 14). Similar to the CHAPS trending (section 8.2.5), the stability follows a very similar trending for the BSA lots across both collections of raw materials (compare Conditions #16, #17, #18, and #19 to Conditions #22, #23, #24, and #25, respectively; FIG. 14). The gauge analysis indicates that BSA lot “A” (Universal Grade, L/N 692) and BSA lot “E” (Diagnostic Grade, L/N 454) yielded the optimal results with respect to long-term stability trending across the entire experiment (FIG. 16B).

An interaction analysis indicates that there is likely to be some interaction between the CHAPS and BSA with regard to stability performance. The CHAPS lot #1 appears to be much more sensitive to substitutions of BSA lot whereas the CHAPS lot #7 seems more robust to BSA lot substitutions (Compare the encircled data points in FIG. 16C). A similar trend can be observed when the individual lot of CHAPS #2-#6 are compared between BSA lots A and lot F (FIG. 16C). This result suggest that there could be a need for a material qualification for BSA (similar to TM-008) when switching lots of CHAPS lot to account for potential interactions between CHAPS and BSA in the calibrator diluent.

The effects of varying the lots of CHAPS and BSA relative to each other in this study are also shown in FIG. 17. Comparing formulations across and within a particular row indicate the effects of substituting lots of CHAPS while keeping the BSA lot number constant. Comparing formulations up and down a given column indicate the effect of substituting lots of BSA while keeping the CHAPS lot number constant. In general, CHAPS lots #3 give the best stability with BSA lot A, whereas CHAPS lot #4 give the best stability with BSA lot E (see solid orange boxes in FIG. 17). In general, BSA lot E seems to give the best stability with both CHAPS lots #1 and #7 (see hatched orange boxes in FIG. 17). In some comparisons (e.g., within BSA “A” comparison with CHAPS lot #1 and CHAPS lot #7; purple hatched boxes in FIG. 17), the raw materials used in the formulation of the Blue Team of calibrators gave higher percent stabilities, perhaps as a result of the different grade of water used in formulation.

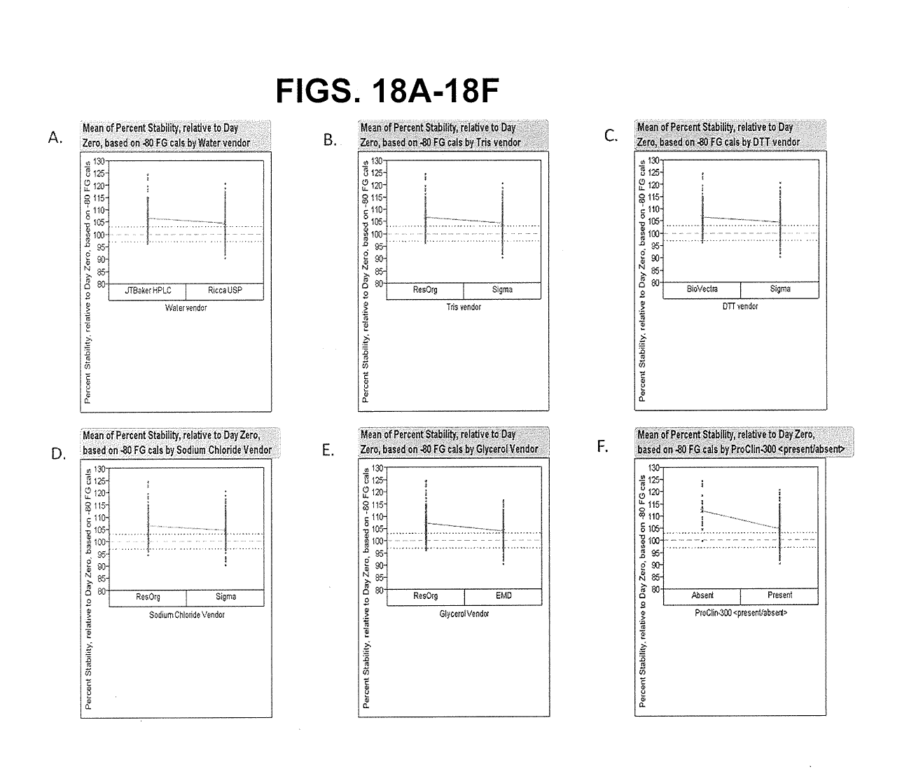

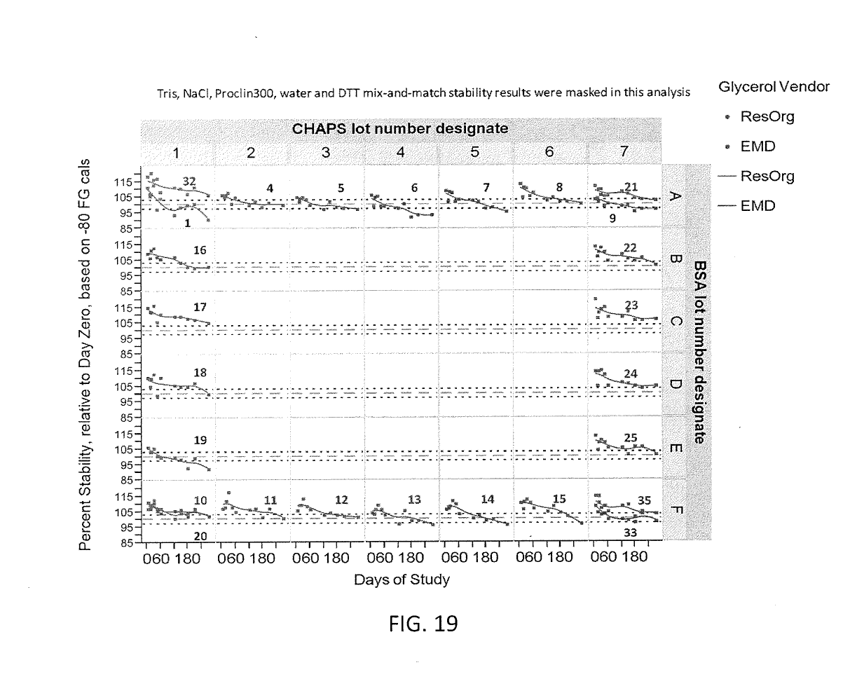

A comparison of the remaining raw material substitutions with regard to long-term stability are shown in FIG. 18A-18F. The effects of the individual raw materials water, Tris buffer, sodium chloride, DTT, water and glycerol all appear to co-vary with respect to whether they are on the Red Team or the Blue Team rather than by vendor per se (FIG. 18A-18E). JMP modeling of the data set resulted in effectors with higher than allowable Variance Inflation Factors (VIF; data not shown). As a result of the nature of the confounding variable with the water situation, other co-varying raw material effectors (e.g., the glycerol vendor; FIG. 19), that track with the Red/Blue team show similar gauge analysis responses (FIG. 18A-E). In fact, substitution of the standard EMD glycerol seems to improve the performance of the Blue Team of raw materials (compare Condition #32 to Condition #1, FIG. 14), and the reciprocal analysis of substituting the test glycerol into the context of Red Team shows worse performance (compare Condition #33 to Condition #35, FIG. 14). This may demonstrate an important role for glycerol in maintaining calibrator stability. The grade of glycerol used in the calibrators may deserve some attention. Another potentially confounding variable is the water grade due to the fact that the water was used to formulate all the stock solutions within a given set of raw materials and makes up ˜70 v/v of the total reaction volume. Experiments are currently in progress that will address more directly the effects, if any, of substituting different grades of water.

On the other hand, the presence or absence of the ProClin-300 shows a different trending pattern (FIG. 18F) relative to the other co-varying raw materials. The absence of ProClin-300 was tested in both the context of the standard raw materials for both the Red Team and the Blue Team. Condition #36 was the Blue Team of raw materials without any ProClin-300 added, and it was clearly the formulation condition with the worst short-term and long-term stability of the thirty-six calibrator formulations (FIG. 14 and FIG. 15A).

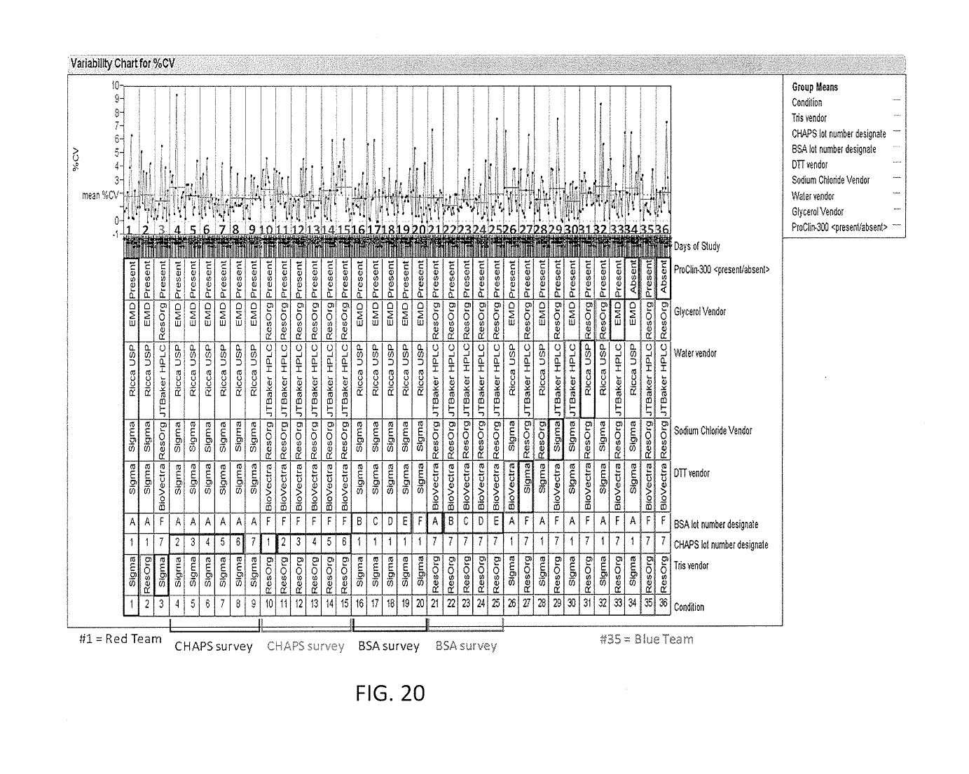

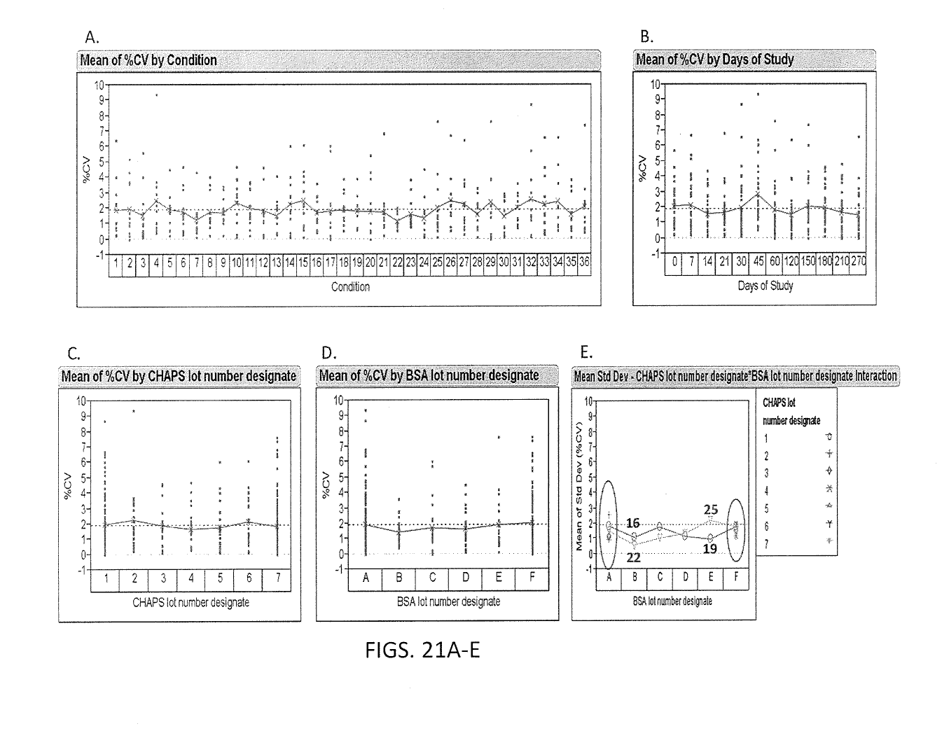

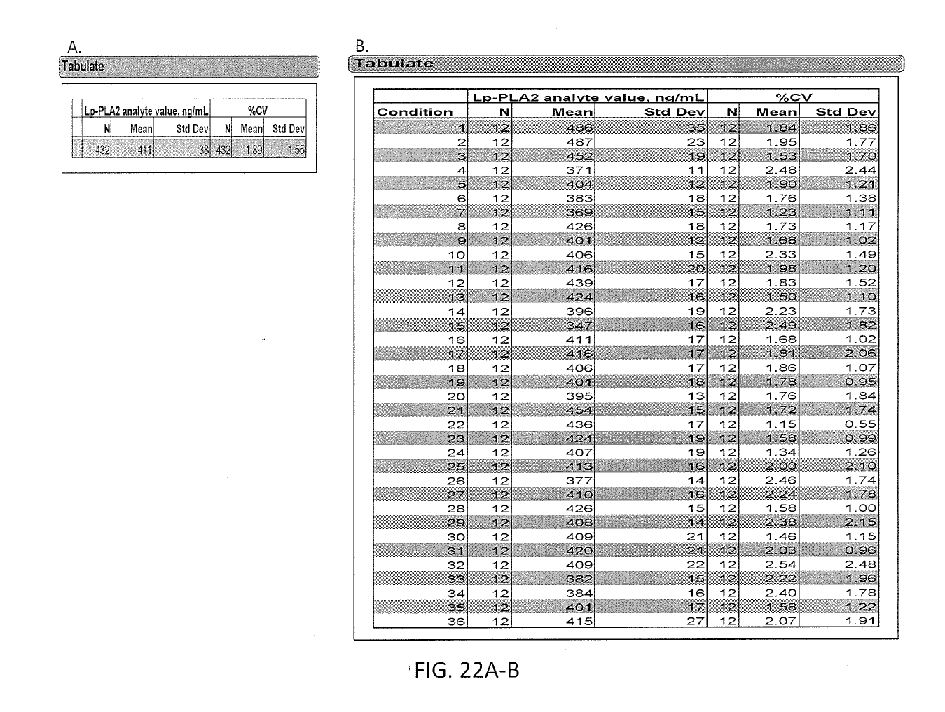

The precision of the thirty-six formulations was for each of the thirty-six calibrator diluent formulations for first nine months of timepoints, including the Day 0 time point, is shown in FIG. 20. The % CV of each of the twelve timepoints (n=2 replicates/time point) are plotted temporally and as a function of formulation condition (FIG. 20). The grand mean of all the % CV measurements for the indicated timepoints across all formulations was 1.89% (FIG. 22A). While the grand mean of all the % CV measurements was excellent, there were clear differences in the individual mean % CV's between the thirty-six formulations as well as the standard deviations of the twelve individual CV's measurements for the thirty-six formulations. The gauge analysis shown in FIG. 21A-21E shows the individual breakouts for the trending by formulation condition, day of the study, and selected effectors (CHAPS lot, BSA lot, CHAPS/BSA interactions). Conditions #7 and Condition #22 showed the best overall precision (i.e., lowest % CV's) at 1.19% and 1.20%, respectively (FIG. 21A and FIG. 22A). However, Condition #22 had the best overall standard deviation of the twelve individual CV's measurements at 0.55%, which was a full 1.00% improvement over the average for all thirty-six formulations (FIG. 22A and FIG. 22B). Condition #31 had the second best standard deviation for the twelve individual CV's measurements at 0.90%, a 0.65% improvement over the average for all thirty-six formulations (FIG. 22A and FIG. 22B). When precision was trended as a function of time, only Day 45 showed noticeably worse precision than the other timepoints (FIG. 21B)

Of the raw materials surveyed in this study, only the CHAPS and BSA lots showed differential effects on precision (FIG. 21, FIG. 21 and data not shown). CHAPS lots #3, #4 and #7 all had better % CV's than the grand mean % CV (FIG. 21C), and BSA lots B, C, and D all had better % CV's than the grand mean % CV (FIG. 21D). An interaction analysis of the [CHAPS lot*BSA lot] for the standard deviation of the twelve individual CV's measurements indicates that the optimal material variation for precision is the combination of [CHAPS lot #7*BSA lot B], a combination corresponding to Condition #22 (FIG. 21E). Swapping out the CHAPS lot (compare Condition #22 to Condition #16; FIG. 21A) or the five other BSA lots show these synergistic effects (compare to Condition #22 to Conditions 21, 23, 24, 25 and #35; FIG. 21A; for descriptive statistics, see FIG. 22B).

Taken together, these results suggest that different raw material combinations can have a positive effect on calibrator precision, and the main driver of optimal calibrator precision appears to be the combination of the particular CHAPS detergent lots and BSA lots utilized. In addition to a synergistic effect on stability (Section 8.3.7; FIG. 16C), these two effectors seemingly work in concert to have a synergistic effect on precision as well (FIG. 21E).

### Example 3

### Response Surface Design DOE Study

Purpose: To assess in detail the effects of varying the concentrations of selected raw materials on calibrator performance in the context of a designed experiment.

Materials:

PLAC ELISA kit, P/N 90123, L/N 1102163;

Antigen: P/N 26203, L/N 1010057;

Tris Base: Sigma P/N T1503, L/N 031M5413V;

CHAPS: Sigma P/N C3023, L/N 018K53003;

BSA: Millipore P/N 81003, L/N 692 (current Production lot);

DTT: Sigma P/N D0632, L/N 031M1753V;

NaCl: Sigma P/N 59888, L/N 040M0225V;

Water: JT Baker P/N 4218, L/N J45E01;

ProClin 300: Supelco P/N 48914-U, L/N LB82798;

EDTA: Fluka P/N 003777, L/N BCBD4995V;

Millipore Steriflip Express Plus, P/N SCGP00525, L/N MPSF006562;

Experimental Procedures:

Experimental Plan. An earlier development report, DR-00133, described the results of a two-level full-factorial design for four potential raw material effectors of calibrator stability in a short-term refrigerated stability study. The four effectors were the pH of Tris-HCl buffer, [7.40, 8.00]; NaCl, [0.154 M, 2.857 M]; CHAPS concentration, [0 mM, 4.76 mM]; and, EDTA concentration, [0 mM, 0.5 mM]. In addition, a fifth, categorical effector was also surveyed: Reducing Agent Identity [None, DTT 0.95 mM, TCEP 0.95 mM]. The standard calibrator diluent concentrations are underlined, and the optimal conditions trended towards the underlined (i.e., standard) concentrations. In addition to the high salt concentration, the inclusion of both the standard concentrations of both DTT and CHAPS was particularly important for calibrator stability. The role of pH in short-term calibrator stability was less clear and seemingly context-dependent.

In this response surface experimental design, the sodium chloride and reducing agent identity were kept constant, and the concentrations of the protons (i.e., pH), buffer concentration, DTT and CHAPS were surveyed using an optimization technique known as a response surface design (specifically, the RSD is a rotatable central composite design). Because central composite designs contain design points from a two-level factorial design (augmented by center points and numerous axial points), they are useful for sequential experimentation. With these considerations in mind, the midpoint and factorial points represented standard raw material concentrations, and the axial points represented opportunities to screen for raw material concentrations that result in either stability improvements or “test-to-failure” outcomes.

The DTT and CHAPS concentrations were explored both above and below their standard concentrations, 0.95 mM and 4.76 mM, respectively. The higher concentrations surveyed were performed with the possibility in mind of enhancing calibrator long-term stability. Conversely, the lower concentrations of each raw material were surveyed in an attempt to “test to failure”.

The pH of the calibrator diluent reagent was surveyed within a relatively narrow titration window [7.80, 7.87, 7.95, 8.05, and 8.18] as part of a targeted optimization effort focused on improving long-term stability.

A survey of higher Tris buffer concentrations was performed to screen for potentially beneficial effects on long-term stability.

Experimental Details. Concentrated stock solutions were created for the two groups of the following raw materials: CHAPS (0.100 M), DTT (1.00M), sodium chloride (5.00M) and Proclin-300 (10% v/v). The Tris base concentrated stock solutions (1.00 M) were created at five different pH's (8.45, 8.25, 8.15, 8.07 and 8.00) to mimic a process in which the starting pH is intentionally set 0.20 pH units more alkaline than the desired final pH prior to the addition of the BSA. All the raw materials were formulated using the JTBaker HPLC grade water. For purely technical reasons, the Millipore BSA (lot 692) was added as powder to each formulation. Briefly, nine separate Buffer A mastermixes, representing the nine buffer concentration/pH combinations, were equilibrated with the common BSA/ProClin-300 components in an isotonic solution. Separately, twenty-six individual Buffer B master-mixes were formulated with the DTT/CHAPS components in a high-salt, buffered solution. A pH adjustment was performed with hydrochloric acid/sodium hydroxide to achieve the intended final pH for each of the fifty-two conditions. After mixing, each formulation was filtered using a 50 mL Millipore Express Plus filtration unit followed by the addition of the recombinant protein Lp-PLA2 to a final analyte concentration of approximately 250 ng/mL, as described in the section above. The calibrator formulations were put on stability at refrigerated temperature. Stability timepoints were taken on Day 0, Day 3, Day 8, Week 2, Week 4, Week 8, Month 3, Month 4, Month 5, and Month 6 using the PLAC ELISA kit.

Results

Response Surface Design DOE Study, Six Months

Previously, a full factorial design DOE was described in which extreme levels of selected raw materials concentrations in the calibrator were screened, including buffer pH, sodium chloride concentration, and the inclusion/exclusion of CHAPS detergent and choice of reducing agent (or none at all). This Response Surface Design DOE study was a follow up study with the goal of optimizing the concentrations of selected standard raw materials in the calibrator diluent, including CHAPS, the reducing agent DTT, Tris buffer concentration and Tris buffer pH.

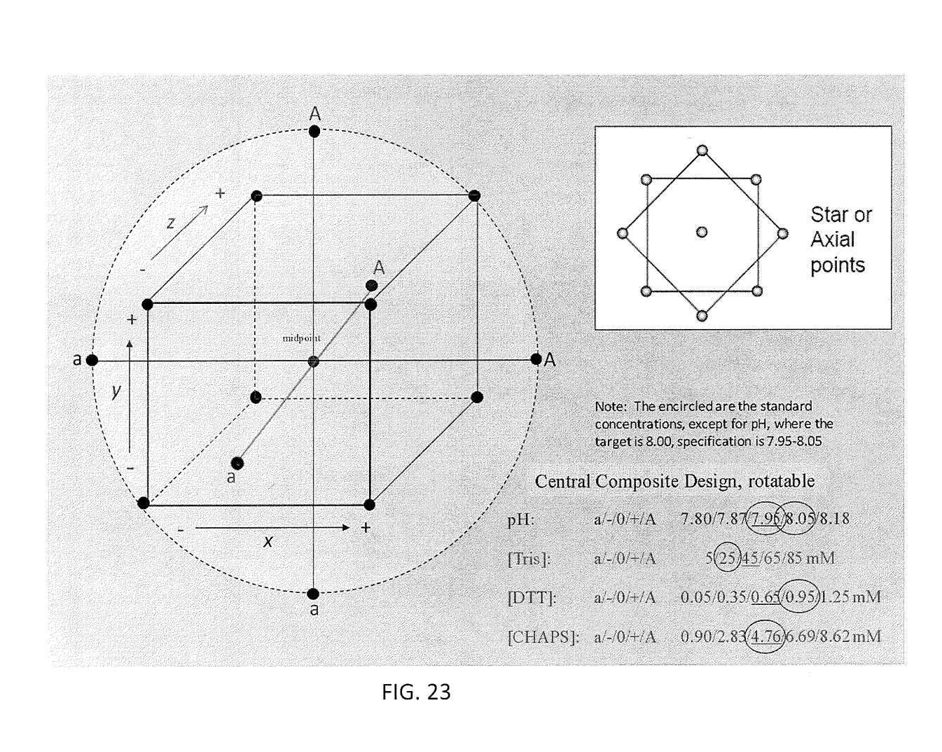

A response surface design is a type of designed experiment that uses a second-degree polynomial model to obtain an optimal response. A central composite design is a particular type of response surface design that contains an imbedded factorial design with center points that is augmented with a group of “star points” (also known as axial points) that allow an estimation of curvature (FIG. 23). The star points are at some distance from the center based on the properties desired for the design and the number of factors in the design. The star points establish new extremes for the low and high settings for all factors and are surveyed in conjunction with the midpoint concentrations of the other effectors. The presence of these axial, or “star points”, is one of the characteristics that distinguishes the central composite design from other types of response surface designs, such as a “Box-Bhenken” design. A full description of the response surface design can be found in the legend to FIG. 23.

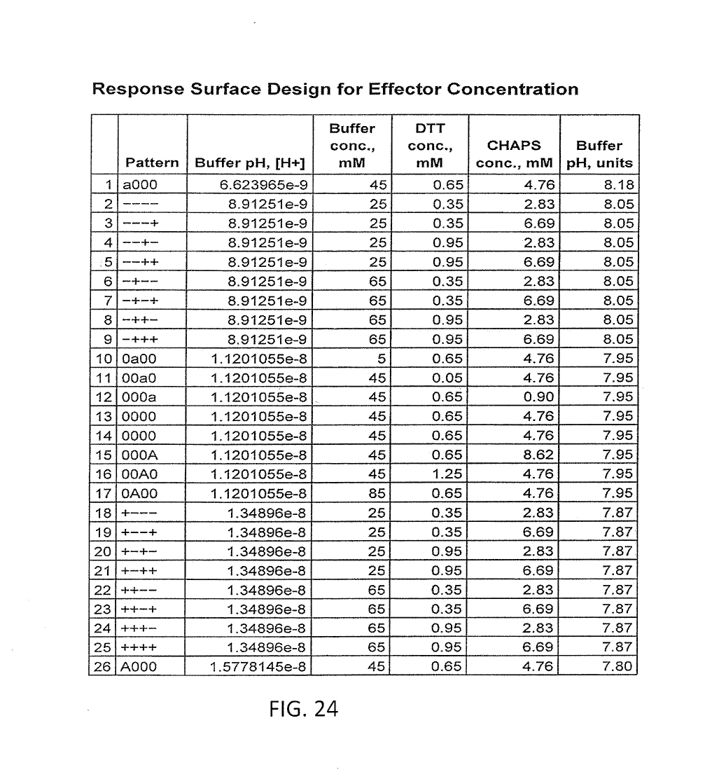

A total of twenty-six formulation conditions were surveyed in this experimental design including a duplication of the center point formulation. Sixteen formulation conditions are contributed by the requirements of the full factorial design (24), which are represented by the sixteen vertices of the factorial design (i.e., in four dimensions). The remaining eight conditions are represented by the two axial positions of each of the four raw materials. The net result is five concentrations are tested for each raw material in various contexts of this type of design (FIG. 23). A full description of the individual formulation conditions is described in FIG. 24.

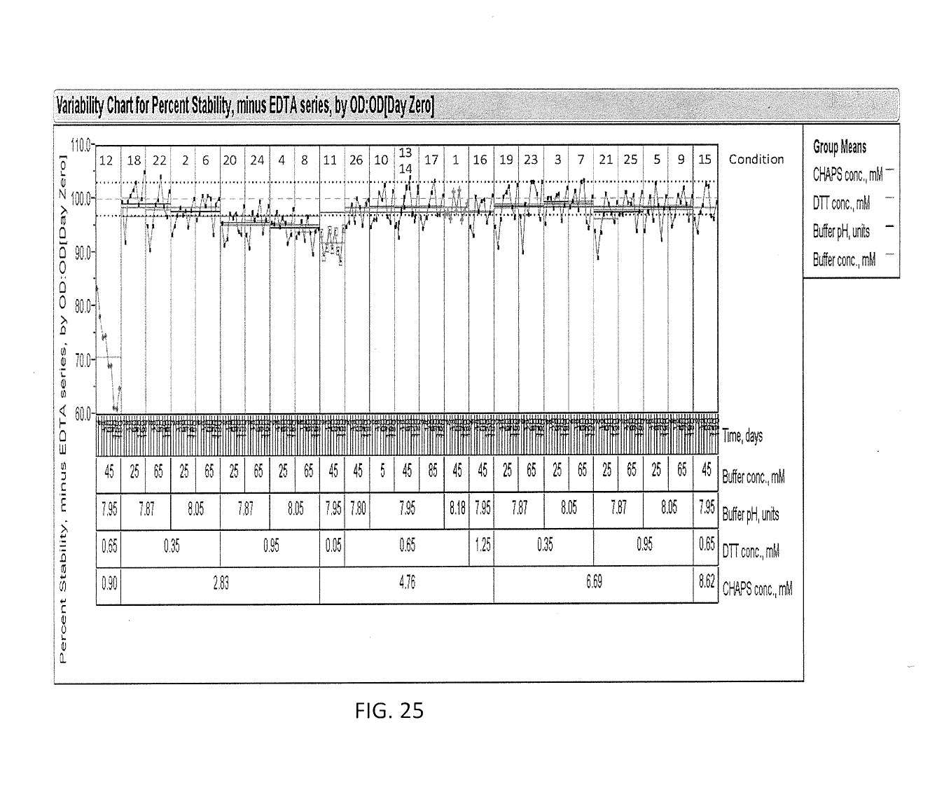

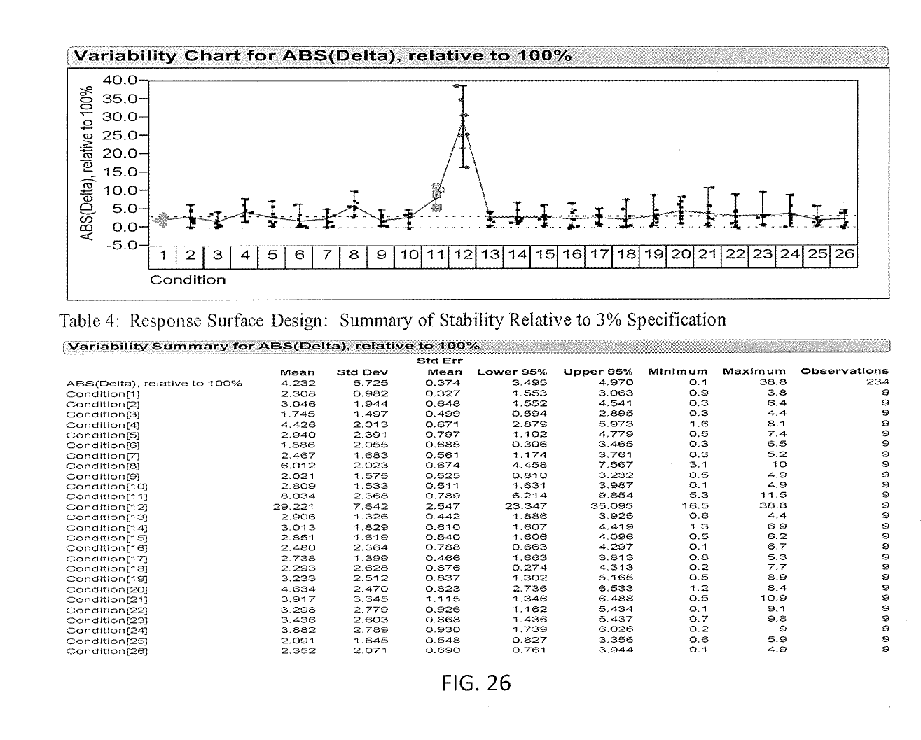

The long-term stability results are shown for multiple timepoints, taken over the course of six months duration (FIG. 25). Stability was calculated based on optical density measurements relative to the same measurement on Day 0 of the study. Several of the conditions showed excellent stability depending on the metric used for stability. Condition #3 had the lowest mean difference in percent stability for the nine timepoints relative to achieving the 100%+/−3% specification at 1.745% (FIG. 26, FIG. 24). This is expressed as the mean of the absolute value of the percent stability difference for each of the individual timepoints relative to 100% stability in FIG. 26. Condition #1 had a smallest standard deviation (0.982%) and the lowest upper 95% confidence interval (+/−3.063%) for the nine timepoints' percent stabilities. This formulation had the highest pH surveyed (i.e., pH 8.18) and encoded by an axial point in the design (FIG. 26, FIG. 24). Condition #25 was the only formulation to have eight out of the nine timepoints meet the +/−3.0% specification.

The three formulation conditions that demonstrated the worst stability were Conditions #8, #11 and #12. These three conditions did not score even a single data point from any of the nine timepoints within the +/−3.0% specification (FIG. 26). Clearly, the worst performer was Condition #12, the axial concentration with lowest CHAPS detergent concentration surveyed (namely, 0.90 mM CHAPS, or about 19% of the standard concentration) with the lowest mean stability of the twenty-six conditions (FIG. 24) and the stability got worse over time (FIG. 25). The second worse formulation for long-term stability was condition #11, the axial concentration with the lowest DTT concentration surveyed (namely, 0.05 mM DTT, or about 5% of the standard concentration). Condition #11 had the second lowest mean stability (FIG. 24) of the twenty-six conditions. Interestingly, the stability of the Condition #11 formulation did not appear to worsen over time (FIG. 25), as there was just an apparent 10% drop-off in stability relative in the first ten days of the study to the initial (day 0) time point.

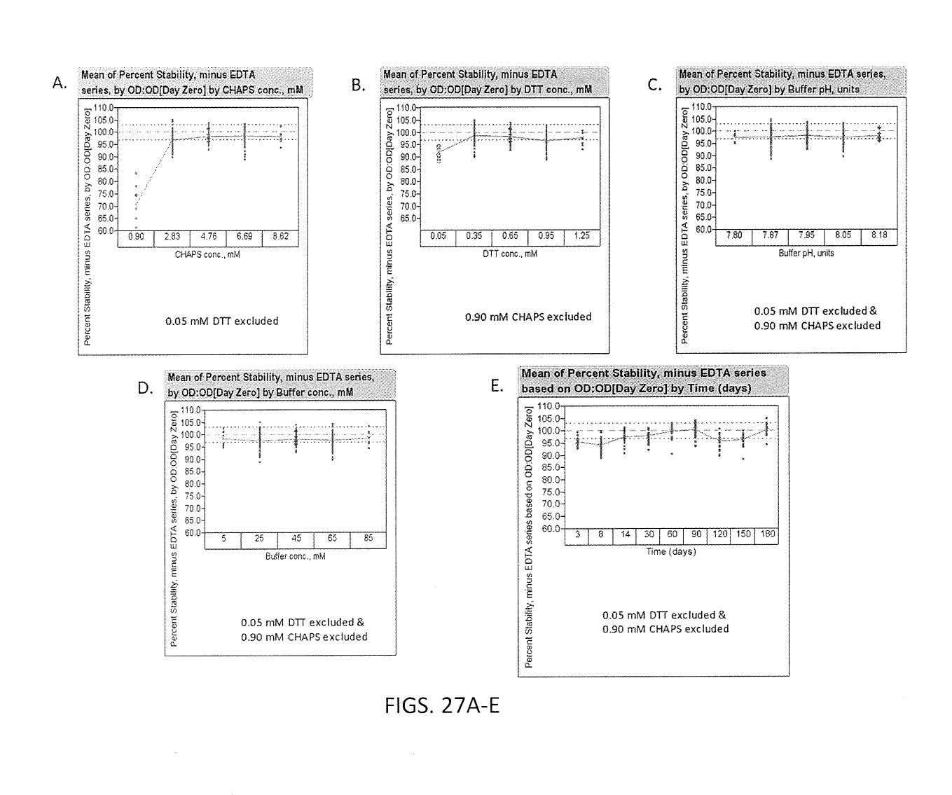

The trending of the gauge analysis for the four effectors studied here shows concentration-dependent effects for two of the raw materials. Consistent with the results of the “Detergent Comparison Study” (e.g., see FIG. 10D), there is noticeable drop in stability when the CHAPS concentration is lowered from the standard concentration of 4.76 mM to 2.83 mM and an even sharper drop in stability when the CHAPS concentration is lowered to 0.90 mM (FIG. 27A). The stability performance seems to plateau at CHAPS concentrations above 4.76 mM, at least within the six month timeframe analyzed here. There is also a noticeable drop in stability when the DTT concentration is drops below 0.35 mM to the next surveyed concentration of 0.05 mM (FIG. 27B). The standard concentration used is 0.95 mM. There may be a peak in the stability response to DTT somewhere around the midpoint concentration of DTT tested (0.65 mM; FIG. 27B). Neither the Tris buffer pH (FIG. 27C) nor the Tris buffer concentration (FIG. 27D) demonstrated any noticeable trending within the six month timeframe analyzed here. There was some day-to-day variability within a total range of approximately 5% in the measured OD values relative to Day Zero (FIG. 27E), but the gauge analysis of the measurements from day-to-day did not follow any discernible trend.

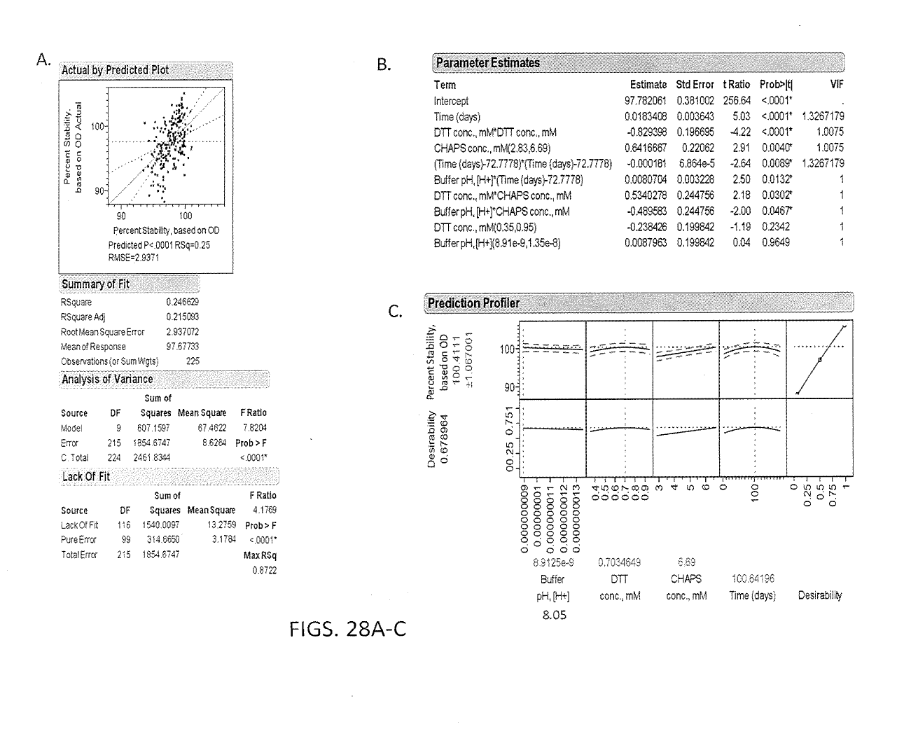

The data was analyzed using the JMP “Response Surface” functionality to fit the data to a model which included the four raw material concentration and time as effectors. The nine points after Day Zero were modeled using time as one of the effectors and eliminating the condition with the lowest concentration of CHAPS (0.90 mM) from the model too much sensitivity was lost when it was included. The model was refined by removing effectors and second-degree interactions sequentially that were not statistically significant (p value <0.05) as described in the legend to FIG. 28A-28C.

The modeling using of the data should be interpreted with several caveats. The model had both a marginal R-squared value (0.246) and a statistically significant lack of fit (p value <0.0001; FIG. 28A)

On the other hand, the Analysis of Variance (ANOVA) for the model was statistically significant (p value <0.0001) and the F-ratio was acceptable (F Ratio=7.82; FIG. 28A)).

Numerous parameter estimates, including several quadratic interactions showed statistical significance and excellent VIF's (FIG. 28B):

Time, p<0.0001

[DTT*DTT], p<0.0001

CHAPS, p=0.0040

[Time*Time], p=0.0089

[pH*Time], p=0.0132

[DTT*CHAPS], p=0.0302

[pH*CHAPS], p=0.0467

The prediction profiler functionality in JMP was utilized to predict the optimal raw material concentrations for the statistically significant effectors. The Buffer pH was kept in the refined model even though it was not statistically significant itself because pH showed a statistically significant quadratic interaction with the effector, Time.

Buffer pH trends toward pH 8.05 (FIG. 28C, panel 1). This pH falls within the standard pH specification of 7.95-8.05 for the calibrator diluent formulation. The quadratic interaction of buffer pH with time (i.e., [pH*Time]) was of modest statistical significance (p=0.0132)

DTT concentration trends toward 0.70 mM (FIG. 28C, panel 2). The standard concentration used in the formulation is 0.95 mM. Notably, the DTT itself was not statistically significant, but the quadratic interaction of [DTT*DTT] showed excellent statistical significance (p<0.0001). In addition to the quadratic interaction of DTT with itself, the DTT concentration also showed a quadratic interaction of modest statistical significance (p=0.0302) with the detergent CHAPS (i.e., [DTT*CHAPS]).

The CHAPS concentration trends toward 6.69 mM (FIG. 28C, panel 3) and showed good statistical significance (p=0.0040). This concentration is about 40% higher than the standard concentration of 4.76 mM used in the calibrator formulation. In addition to the above-mentioned interaction with the DTT, the CHAPS detergent also showed a quadratic interaction of modest statistical significance (p=0.0467) with the effector, pH (i.e., [pH*CHAPS]).

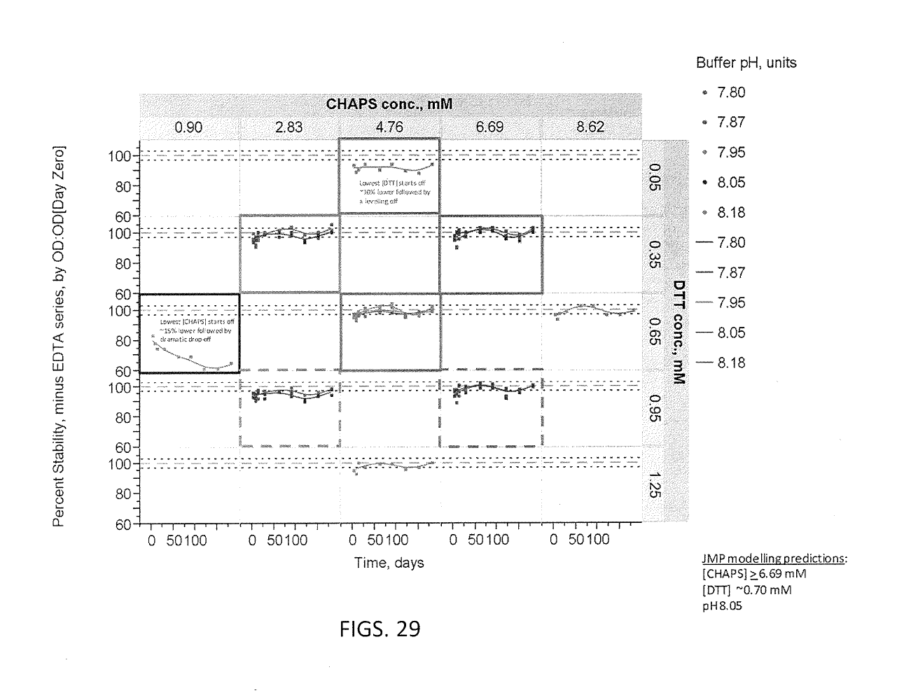

The relationship between the main effectors in the response surface design (CHAPS, DTT and pH) are parsed diagrammatically in FIG. 29. The effects of the test-to-failure, low concentrations (axial) of CHAPS and DTT show obvious deleterious effects on stability (see red box and the blue box, respectively, in FIG. 29). The deleterious effects of the second lowest concentration of CHAPS tested (i.e., 2.83 mM) were selectively observed for the conditions at high DTT concentration (0.95 mM DTT, and the conditions most similar to the those surveyed in the Detergent Comparison Study) and not low DTT concentration (0.35 mM DTT; compare hatched pink box to the solid pink box, respectively, in FIG. 29). At higher CHAPS concentrations, there is no differential stability observed between the high and low DTT concentrations (compare the traces within the hatched and solid green boxes, respectively, in FIG. 29). At higher CHAPS concentrations, the pH 8.05 subtly out-performs the pH 7.87 conditions when the purple traces are compared to the red traces residing within the two green boxes in FIG. 29. On the other hand, at the low CHAPS concentration (i.e., 2.83 mM), the pH 7.87 appear to fare better in stability than the pH 8.05 (compare the red traces to the purple traces within the solid and hatched pink boxes, respectively, in FIG. 29). Consistent with the JMP modeling, very good stability is also shown by Condition #1 (the orange trace in the orange box in FIG. 29), the axial condition representing the highest (most alkaline) pH surveyed in the context of the midpoint concentrations for the other three raw materials. These interactions between [pH*CHAPS] and [DTT*CHAPS] were predicted by the JMP modeling (Section 8.4.7.3), and these differential effects on stability can be visualized using the type of diagram shown in FIG. 29.

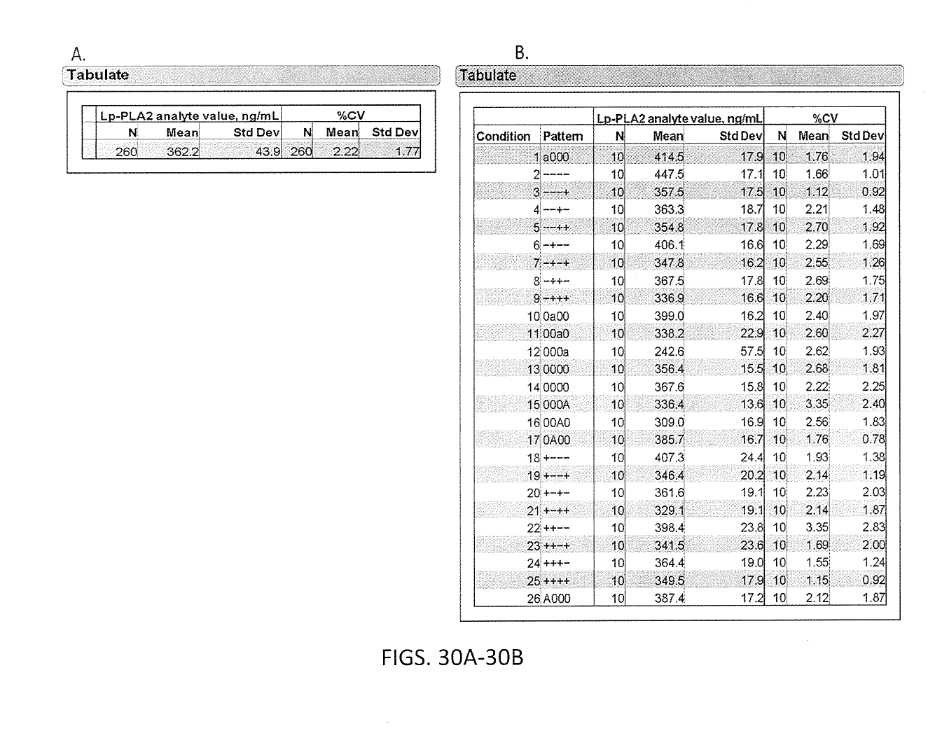

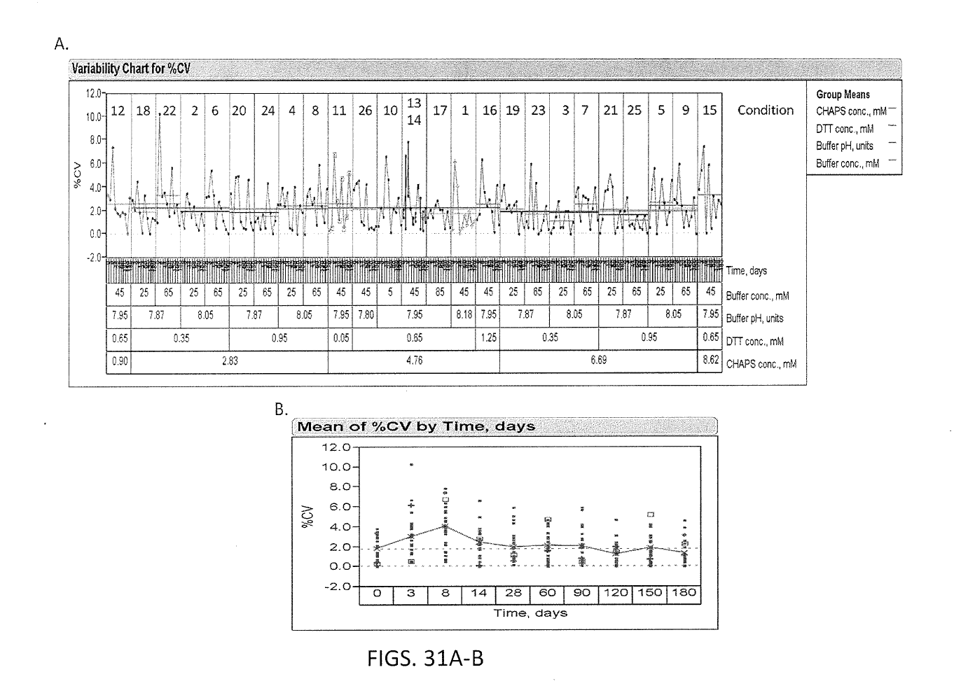

The precision of the twenty-six calibrators was also analyzed as a function of raw material concentration. Imprecision was calculated for each condition at each of the ten timepoints, n=2 replicates per time point. Overall, the precision across the experiment was very good, with a grand mean % CV of 2.22% (FIG. 30A). Condition #3 and Condition #25 had the best overall precision with average % CV's of 1.12% and 1.15%, respectively (FIG. 30B). Both these conditions utilized the 6.69 mM CHAPS concentration (FIG. 31A). Condition #17 (axial point for high buffer concentration) had the best standard deviation of the ten % CV measurements at 0.78% with Conditions #3 and #25 being tied for the second-best measurement at 0.92% (FIG. 30B). Modeling of the imprecision data using JMP gave poor results (data not shown).

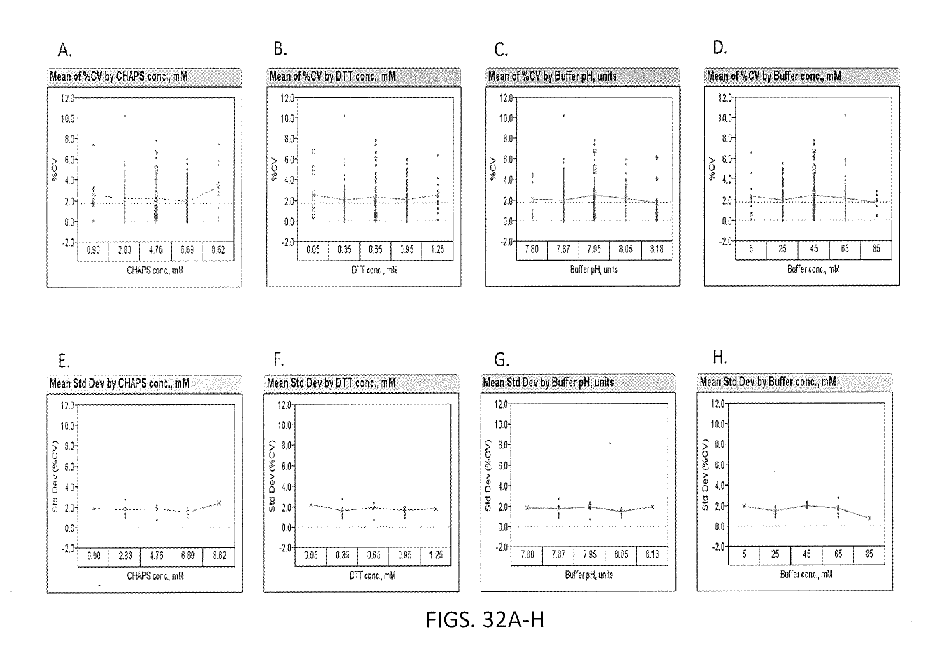

The imprecision of the measurements for all twenty-six conditions was analyzed by gauge analysis, and the mean % CV was at the grand mean % CV on Day 0, but got worse on the next two timepoints on Day 3 and Day 8 (FIG. 31B). The precision measurements stabilized after about 28 days and remained relatively consistent for the remaining five months of the study (FIG. 31B). Additional gauge analyses using both the mean % CV of all the measurements (FIGS. 32A-32D) and the mean standard deviation of the % CV's for the ten measurements for each condition (FIG. 32F-32H) was performed for each of the four raw materials as a function of concentration. In general the second highest CHAPS concentration (FIG. 32A), the highest buffer pH (8.18; FIG. 32C) and the highest buffer concentration showed the best performance by mean % CV (FIG. 32D). Surprisingly, the highest concentration of CHAPS showed the worst performance in terms of mean % CV (FIG. 32A). Using the mean standard deviation of the ten % CV measurements as a metric, the highest CHAPS concentration again showed the worst performance (FIG. 32E), and the highest buffer concentration showed the best performance (FIG. 32H). The DTT concentration showed no discernible trend by either metric (FIGS. 32B and 32F).

Summary: Many of the formulation conditions surveyed here showed excellent calibrator stability performance relative to both the assigned +/−3% stability specification as well as excellent precision. Taken together, the combination of the JMP model fitting analysis of stability, the gauge analysis of both stability/precision, and the consideration of the relative performance of individual formulation conditions in both stability/precision can be used to make some general conclusions. The JMP modeling suggests that optimal stability may be conferred as the CHAPS concentration approaches 6.69 mM and possible synergistic effects between the CHAPS and DTT and the CHAPS and pH. The JMP modeling also suggests a curvature to stability response with respect to the DTT concentration. The JMP predicted an optimal pH trending towards pH 8.05 and the axial point for high pH (8.18) in the experiment yielded excellent stability performance. With respect to precision, the gauge analysis suggest a small, gradual improvement in precision when the CHAPS concentration increased up to 6.69 mM followed by a dramatic worsening in precision performance upon further increasing of the CHAPS concentration to 8.62 mM. There is also a possibility of prospective precision improvements being gained by increasing the pH to 8.18 and/or by increasing the buffer concentration to 85 mM. While promising, it should be noted that these axial buffer formulation conditions were individually tested in the context of the midpoint concentrations of the other raw materials. They were neither tested in combination with each other nor in the context of the optimal CHAPS concentration.

### Example 4

### Buffer and BSA Survey

Purpose: To study the effect of various buffer compositions, pH and process changes as well as various BSA grade/lot substitutions on calibrator stability and precision.

Materials:

PLAC ELISA kit: P/N 90123, L/N 1106130

Antigen: P/N 26203, L/N 1010057

Buffering agents: Tris Base solution (1.0 M solution, titrated with HCl by manufacturer): Teknova, P/N T1080 (L/N 16D1001, L/N 08L1001); Tris Hydrochloride salt (MW 157.6 g/mol); Sigma P/N T3253, L/N 071M5401V; Tris base (MW 121.1 g/mol); Sigma P/N T1503, L/N 031M5413V;

CHAPS: Dojindo P/N C008, L/N DC862

BSA: various grades and lot numbers screened: (1) Millipore “Universal” grade, P/N 81003. Per the manufacturer, this grade is manufactured by a proprietary heat-shock fractionation process, using caprylic acid as an albumin stabilizer. A highly consistent and widely used grade of BSA powder for diagnostic, cell culture and microbial fermentation applications. Assay: Purity 98%-100%, IgG below detectable limits. (L/N 692, L/N 693, L/N 694); (2) Millipore “Diagnostic” grade, P/N 82045. Per the manufacturer, this grade is manufactured by a proprietary heat-shock fractionation process, and this BSA powder is treated to insure inactivation of proteolytic activity. Assay: Purity 98-100%, protease below detectable limits, IgG below detectable limits. (L/N 452, L/N 453, L/N 454); (3) Millipore “Fatty Acid-Free” grade, P/N 82002. Per the manufacturer, this grade is a highly purified BSA powder extensively treated to remove fatty acids. This grade is manufactured by a proprietary heat shock fractionation process. Assay: Purity 98-100%, free fatty acids 0-0.2 mg/g. (L/N 120; L/N 131; L/N 134);

DTT: Sigma P/N D0632, L/N 051M1871V;

NaCl: Sigma P/N 59888, L/N 040M0225V;

Water: JT Baker P/N 4218, L/N J45E01;

ProClin 300: Supelco P/N 48914-U, L/N LB82798;

Glycerol: EMD GX0185-6, L/N 50349114;

Millipore Steriflip Express Plus, P/N SCGP00525, L/N MPSF006562.

Experimental Procedures:

Experimental Plan. The experimental plan includes five different series of buffer conditions regarding the form, concentration, starting pH, and final pH used in the calibrator diluent formulation. A survey of different grades of Millipore BSA grade is also included in the study. Series A involves using a pre-formulated solution from Teknova consisting of 1.00 M Tris base (titrated with hydrochloric acid) to pH 8.00. Included within this series is a titration of Tris concentration up to 125 mM in 25 mM incremental steps. Series B involves using the Tris-Hydrochloride salt form of the buffer, with or without a pre-pH step. A “pre-pH” step refers to a process step in which a starting pH is established following the addition of the buffer and sodium chloride to the formulation. This pre-pH process was first implemented in the context of using the Tris base in the calibrator diluent formulation. Originally, the pre-pH process step was implemented to circumvent a formulation process issue in which the BSA will precipitate between pH 9.0 and 8.5 as the solution becomes more acidic upon titration of hydrochloric acid (This presumably is a consequence of the BSA undergoing a pH-dependent structural transition known as the N-B transition). Series C involves titrating a 1.00 M Tris-Hydrochloride solution and a 1.00 M Tris base solution against each other to achieve the indicated starting pH. Similarly, Series D involves using the Tris base form of the buffer with a pre-pH step and three different starting pH's. Series E is the survey of three different grades and/or lots of BSA obtained from Millipore, prepared according to a standard process. A total of twenty-six different formulations were surveyed in this study. Each calibrator reagent was assayed using a PLAC ELISA kit over the nine timepoints.

Experimental Details. A concentrated master-mix (4×) of the raw materials (CHAPS, DTT, and Proclin-300) common to all formulations in this study was prepared and an appropriate volume was added last to all reactions to achieve the standard final concentrations of these components. Separately, thirty individual formulations reflecting the intended material/concentration permutations for the buffer and/or BSA grade as well as the pH variation for the buffer were prepared. In many cases, a pre-pH step was implemented to establish an initial pH following the addition of the buffer and sodium chloride. After execution of the pre-pH step (if necessary), the indicated grade/lot of BSA was added and mixed. After the addition of the master-mix, the final pH was titrated using hydrochloric acid or sodium hydroxide, as appropriate, for each of the thirty formulations. An appropriate amount of 100% glycerol is added so that the final concentrations of all raw materials are at their standard calibrator diluent concentrations per MP-21090. After mixing, each formulation was filtered using a 50 mL Millipore Express Plus filtration unit followed by the addition of recombinant protein Lp-PLA2 to a final analyte concentration of 250 ng/mL as described in the Experimental Procedures. The calibrator formulations were aliquoted, and each of the thirty formulations was put on stability at both refrigerated temperature and −70° Celsius. Stability timepoints were taken on Day 0, Day 1, Day 4, Week 1, Week 4, Week 6, Month 2, Month 3, and Month 4 using a PLAC ELISA kit.

Results

Buffer and BSA Survey

A comparison of protocols for the calibrator diluent formulation revealed several differences in the raw materials (Tris buffer, water grade), methods (a pre-pH step) and supplies (filtration) used at the two CMO's. A pre-pH step is used when using the Tris base as a starting material for the diluent in order to circumvent a bovine serum albumin protein precipitation issue. The first experimental goal of this study was to compare differences both materials and formulation processes with regard to the Tris buffer. Eighteen different permutations of Tris raw materials, methods, pH, and concentrations were screened for potential effects on calibrator stability and precision. The experimental plan also directly compares the Tris material used in the original development report conditions directly to the current methodologies. The second experimental goal was a more extensive comparison of available Millipore Probumin BSA grades known as Universal, Diagnostic and Fatty Acid-Free (for full description of different grades, see section 5.1.4.5). Three lots of each Probumin BSA grade were tested in the context of the MP-21090 standard formulation process for effects on calibrator stability and precision. A total of twenty-six different formulations were screened in this study.

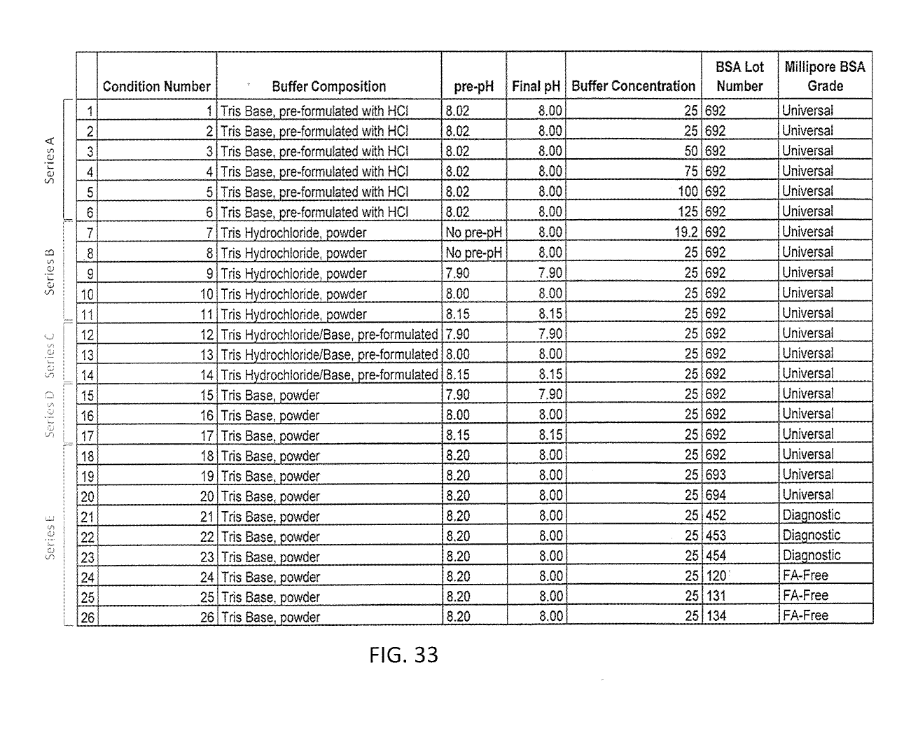

A schematic of the experimental design for the Buffer/BSA survey stability study is shown in FIG. 33. Conditions #1 and #2 are a comparison of two lots of the Teknova Tris buffer product that was used in the original development report to assay stability, used at the standard concentration. Conditions #3-#6 survey higher concentrations, in 25 mM increments, of the indicated Teknova Tris buffer lot used in Condition #2 (FIG. 33). Conditions #7-#11 utilize the hydrochloride form of the Tris buffer, comparing different buffer concentrations and surveying different combinations of with or without the pre-pH step and/or different starting/final pH's (FIG. 33). Condition #8 mimics a current process. Conditions #12-#14 utilize the hydrochloride and base forms of the Tris buffer, titrated against each other at the indicated pH (FIG. 33). Conditions #15-#18 utilize the base form of the Tris buffer, each surveying different combinations of starting/final pH's (FIG. 33). Condition #18 mimics another current process. Conditions #18-#26 survey the substitution of different grades/lots of Probumin BSA into a standard formulation raw materials/process (FIG. 33). Each formulation was put on stability at refrigerated temperature and −70° Celsius and stability was monitored over the course of four months.

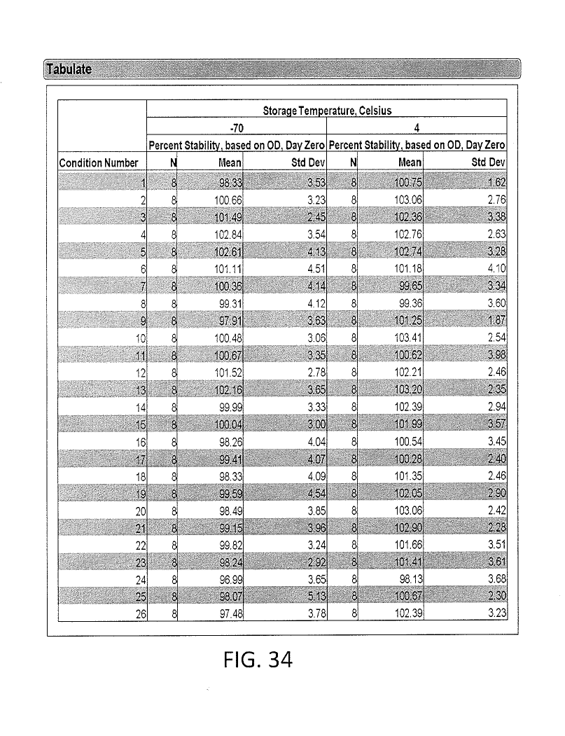

The percent stabilities (mean+/−STDEV) for each of the twenty-six calibrator formulations on stability at two storage temperatures (refrigerated and −70 Celsius) are shown in FIG. 34. In general, the mean stabilities for all twenty-six formulations were very good at both stability temperatures across the eight timepoints. The one formulation condition that particularly stood out was Condition #1 stored at refrigerated temperature with a mean stability of 100.75%+/−1.62% (FIG. 34). Curiously, this high achievement was not replicated by an otherwise identical formulation (i.e., Condition #2; 103.06%+/−2.76%) on stability at the refrigerated temperature. Condition #2 utilized a different lot of Teknova buffer of an identical starting pH to Condition #1 (namely, pH 8.02).

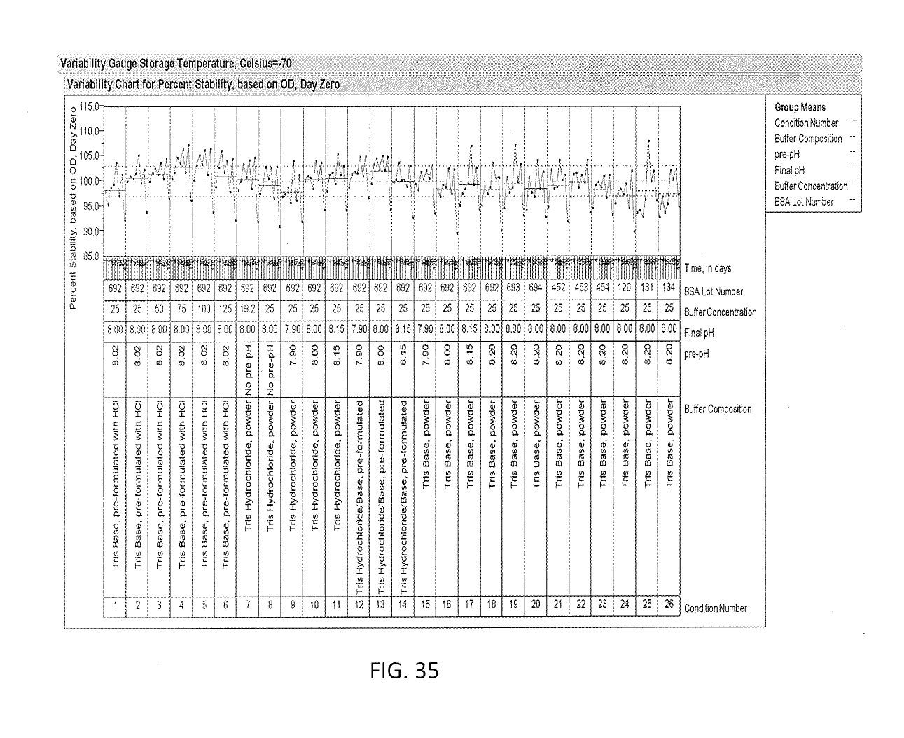

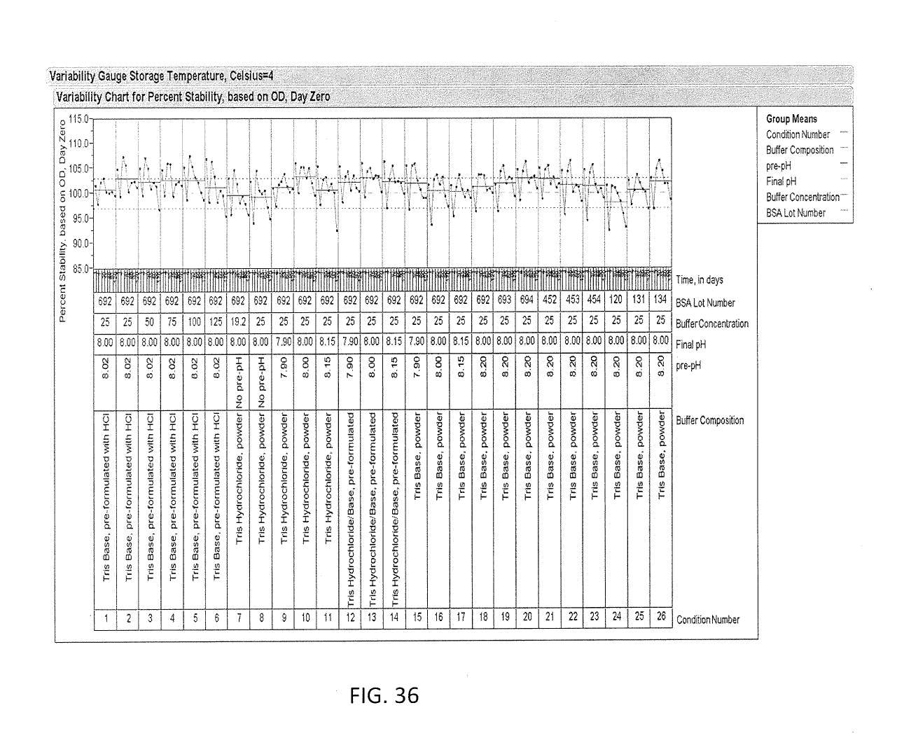

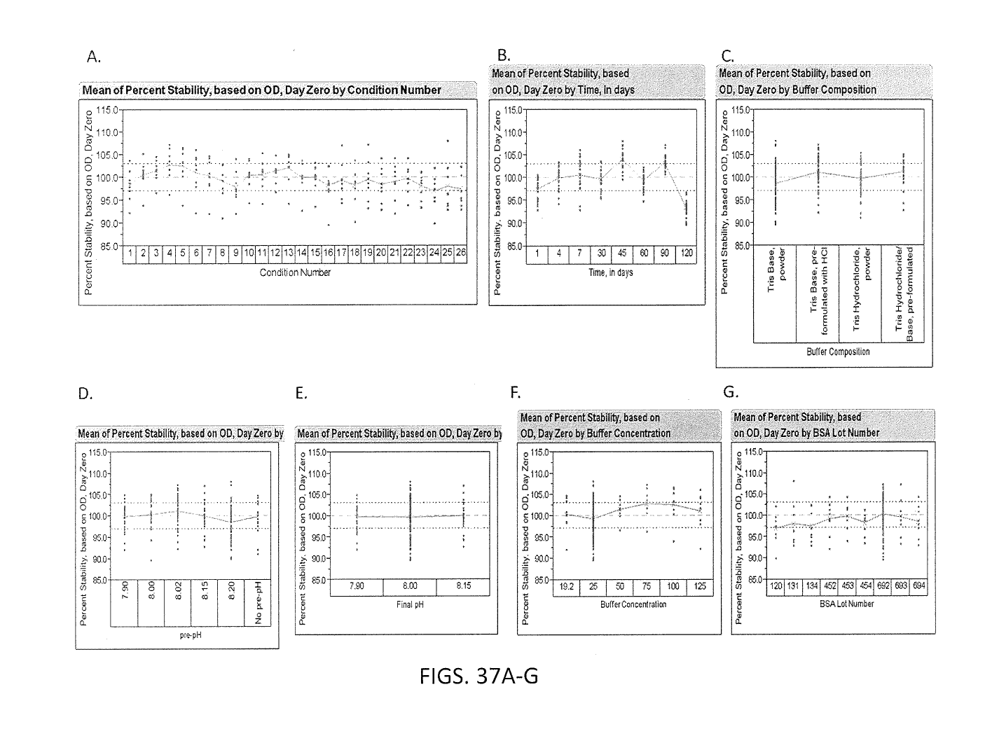

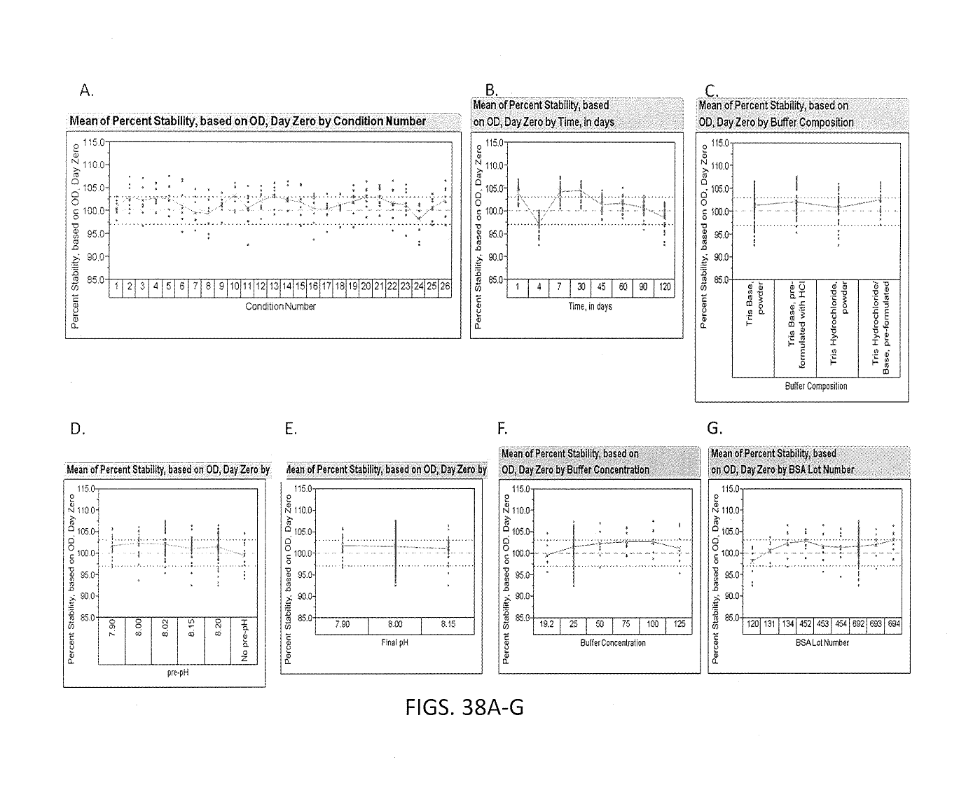

A comparison of the percent stabilities across the initial eight timepoints (Day 1, 4, 7, 30, 45, 60, 90, 120) for the formulations on stability at −70 Celsius and refrigerated temperature are shown in FIG. 35 and FIG. 36, respectively. The gauge analysis shows some interesting trends. The calibrators stored at the two temperatures show very similar trending for buffer composition and buffer process (reflecting small differences in trend lines for pre-pH and final pH), but they show slightly different trending in response to BSA lot number and day of assay (Compare red traces in FIG. 37A-37G to FIG. 38A-38G). Overall, the differences observed are relatively small in magnitude with the trend lines mostly falling within the +/−3% specification with the exception of the day-to-day variation.

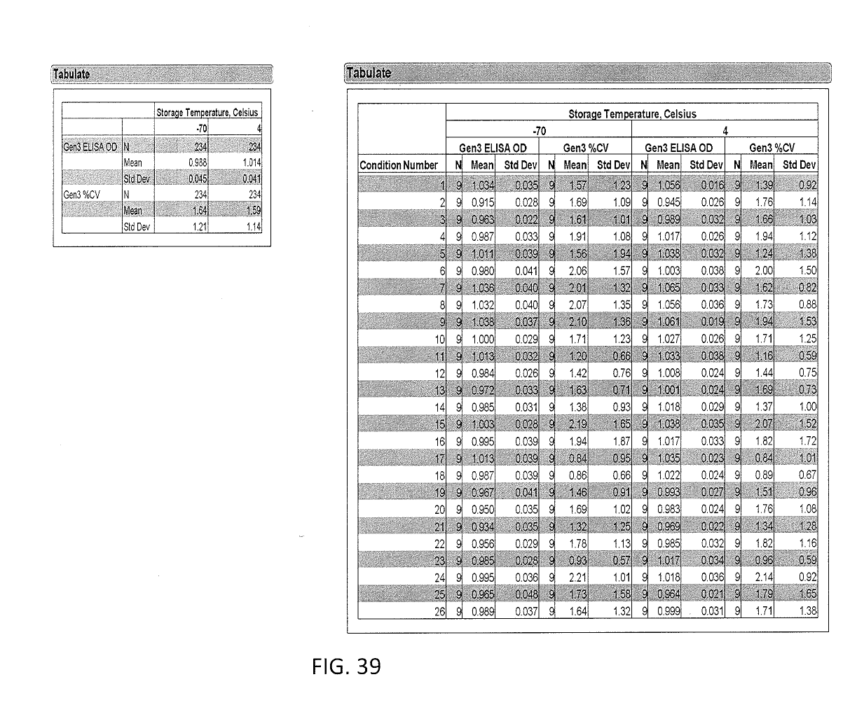

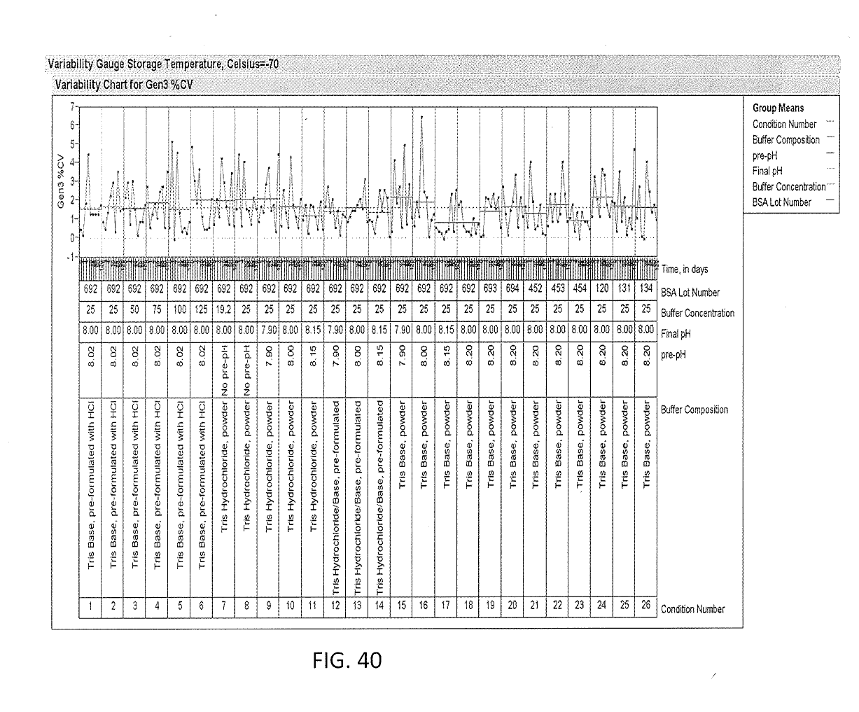

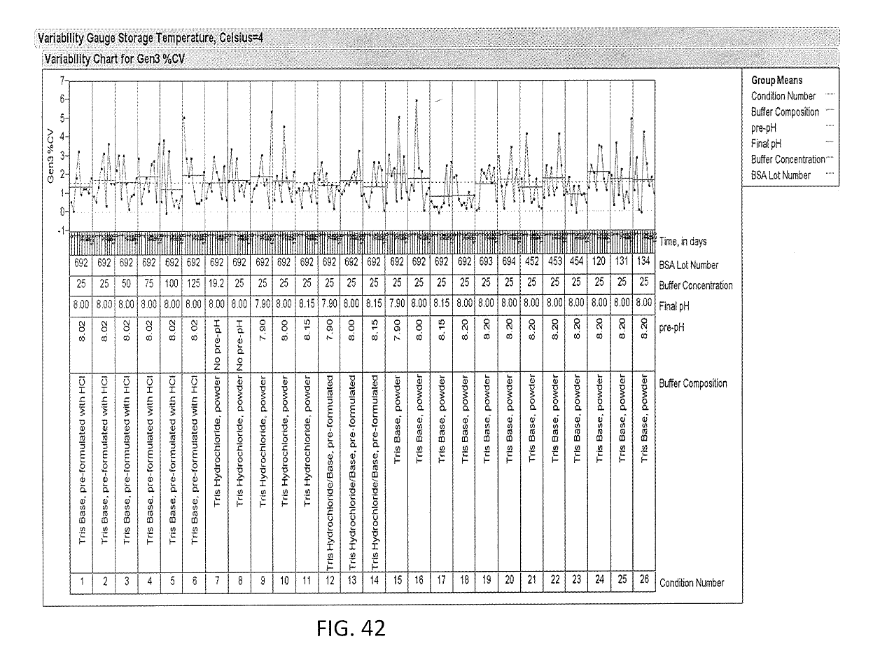

A comparison of the imprecision across all nine timepoints (including the Day 0 time point) showed good congruence between the average precision for the twenty-six formulations across two storage temperatures (FIG. 39). The best formulation for precision was probably Condition #18, which mimics a current formulation raw materials/process. Condition #18 demonstrated a mean % CV of 0.86% (+/−0.66%) and 0.89% (+/−0.67%) at −70 Celsius and refrigerated temperature, respectively. Condition #17, which differs only slightly from Condition #18, was also impressive, with a mean % CV of 0.84% (+/−0.95%) and 0.84% (+/−1.01%) at −70 Celsius and refrigerated temperature, respectively. Condition #11 and Condition #23 also showed promising precision. Condition #11 is identical to Condition #17 but pre-pH's the hydrochloride salt rather than the base form of Tris to the indicated starting and final pH's (FIG. 34). Condition #23 is identical to Condition #18 except it substitutes Diagnostic Grade BSA L/N 454 for the standard Universal Grade BSA L/N 692. A full comparison′ of the precision performance at both storage temperatures, −70 Celsius and refrigerated, for each time point in the first four months of this stability study can be found in FIG. 40 and FIG. 42, respectively.

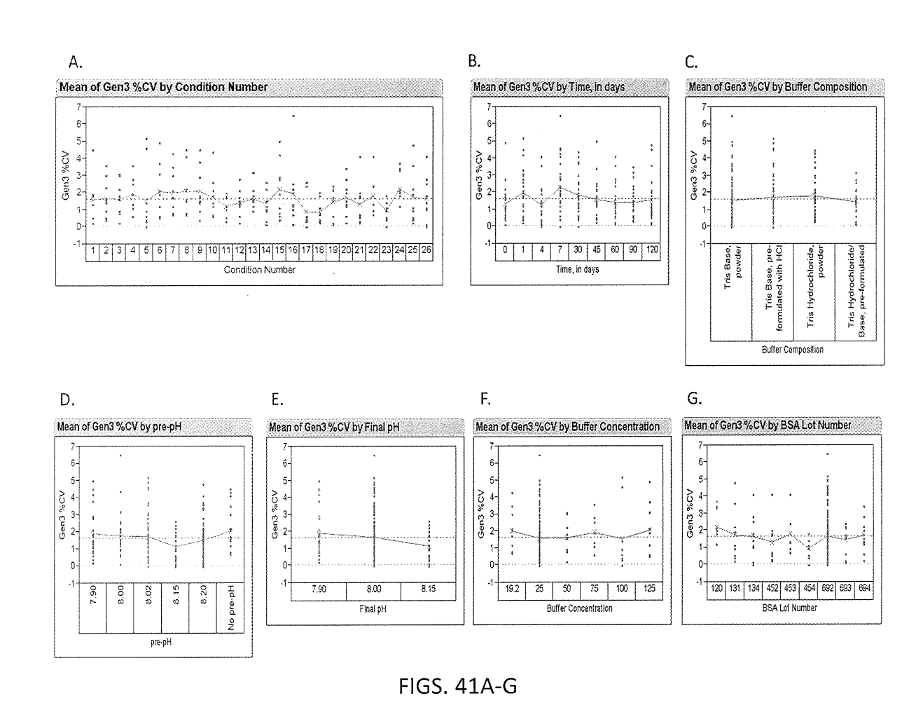

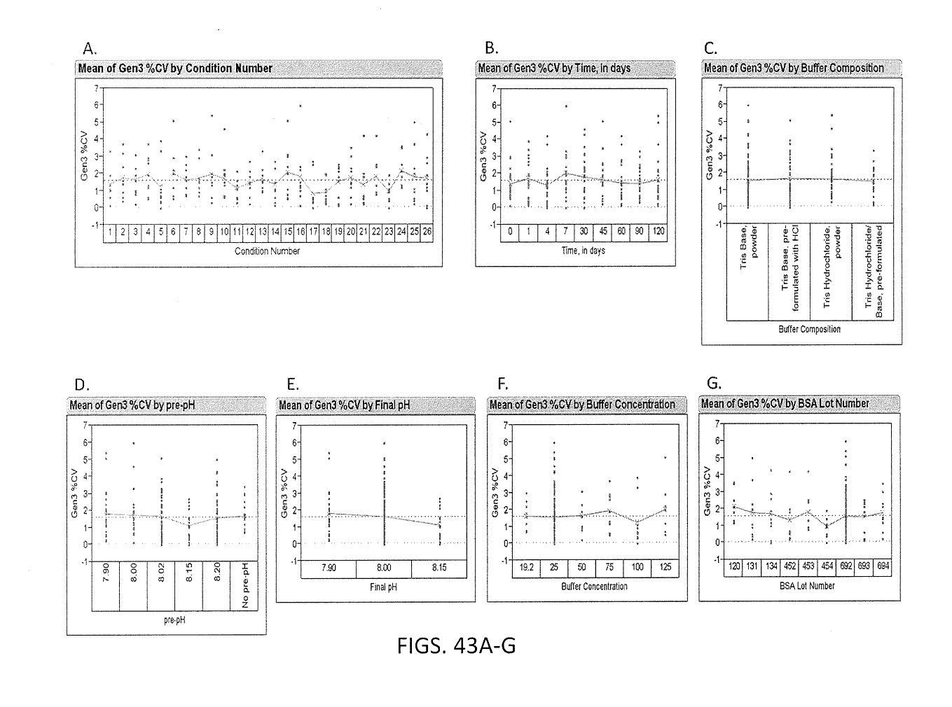

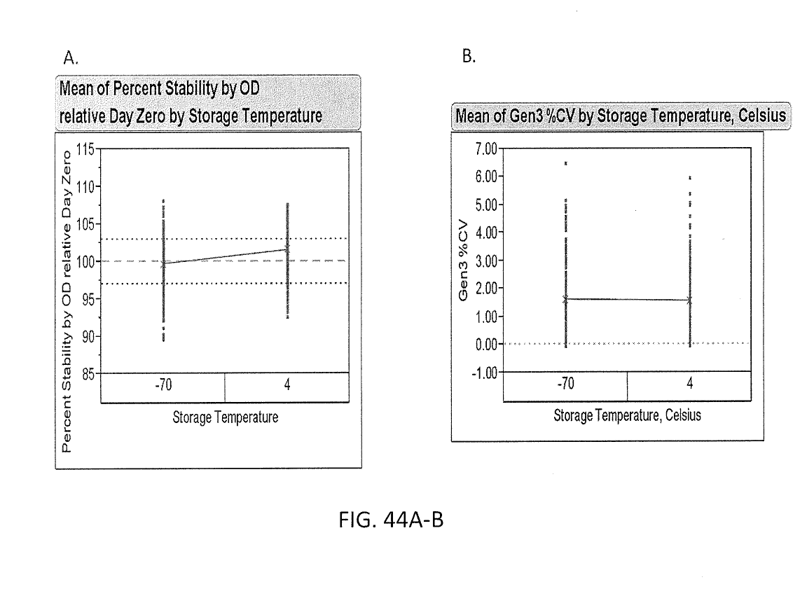

The gauge analysis indicates very similar trending for the precision results at both storage temperatures. The trends when broken out by condition number, time, buffer composition, pre-pH (starting pH), final pH, buffer concentration and BSA lot number show also identical traces for the calibrators stored at both temperatures (Compare FIGS. 41A-41G and FIGS. 43A-43G). The buffer composition shows little effect (FIGS. 41C and 43C), but the starting pH and final pH trend toward pH 8.15 (FIGS. 41D, 41E and FIGS. 43D and 43E, respectively). Buffer concentration did not show any apparent trend (FIGS. 41F and 43F), but BSA lot number does show an apparent trend with respect to precision performance. The BSA trending indicates that the individual lot of BSA, rather than the grade, has the most effect on performance with respect to precision (FIG. 41G and FIG. 43G). Diagnostic Grade Probumin lot number #454 is particularly promising by this metric. Fatty Acid-Free Probumin lot #120 was the worst performer by this metric.

The gauge analysis was also performed across all the calibrator formulations to look at the overall trending of both stability and precision performance as a function of storage temperature. The calibrators stored at −70 Celsius trended right at −100% stability relative to their Day 0 OD value whereas the refrigerated calibrators came in slightly higher at −101%. With regard to precision, there was virtually no difference between the calibrators stored at different temperatures.

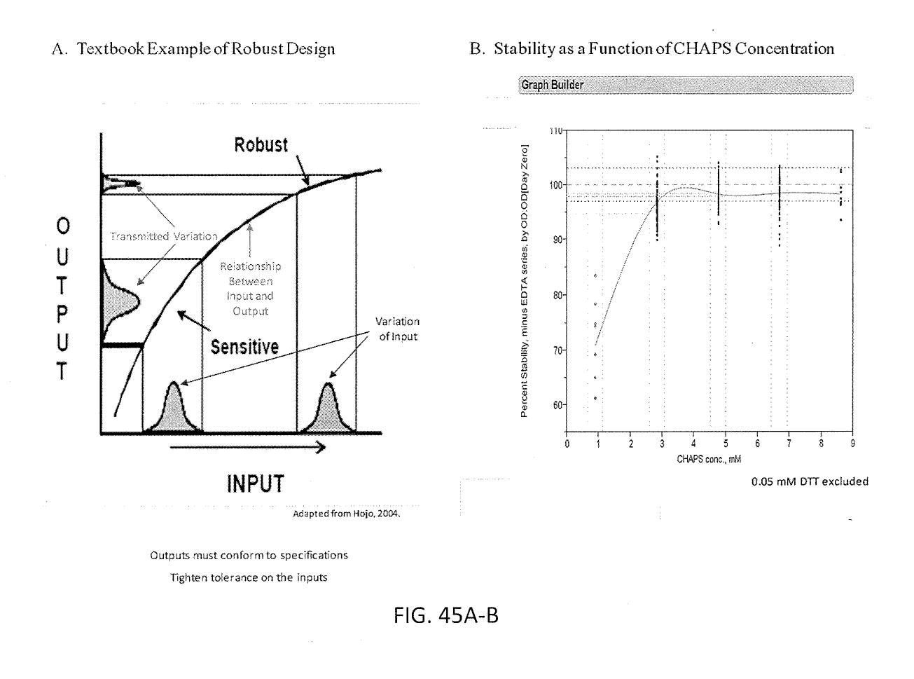

In Examples 1-4, above, a central focus of the continuous product improvement effort is the formulation of a calibrator diluent matrix useful for assays including (but not limited to) ELISA kit calibration standards. For example, the calibrators may cause ELISA kit stability issues if not properly managed. The studies presented here comprise a series of experimental approaches focusing on the formulation of the calibrator matrix and are intended as follow up studies to an initial short-term stability study. In an earlier development report, key inputs for maximal calibrator stability were identified: the inclusion of both the CHAPS detergent and a reducing agent in the context of the standard buffer conditions including the high-salt solution. In the studies presented here, component swapping studies and a designed experiment were used to establish a detailed understanding of the effects of key inputs (raw material variability, raw material concentrations and formulation process) on the outputs (calibrator stability and calibrator precision). The characterization of the effectors suggests optimal target concentrations of certain raw materials that may be useful for a more robust design of the calibrator matrix. This robust design plan may utilize two tactics. The first tactic is to adjust the target concentrations of certain raw materials so that the output (percent stability) is less sensitive to any variability in the input (namely, raw material concentration; see FIG. 45A). A typical scenario is when a response surface design experiment indicates a non-linear relationship between an input and an output. Reducing variation in the output requires re-designing the process/formulation so that the variation transmitted to the output is minimized for a given amount of input variation (See FIG. 45A for a textbook example). The second tactic is to adjust the target concentration of the inputs in such a way as to minimize the variance in the output of the mean stability measurement at a given time point. Since the standard deviation of the measurements is the square root of the variance, the implementation of this approach involves minimizing the coefficient of variation of the stability measurement taken at each time point. The results of these four stability studies suggest practical ways of utilizing both tactics to optimize and improve the performance of a calibrator matrix with respect to both calibrator stability and precision.

Regarding example 1, the detergent comparison stability study, the detergent comparison study was a component swapping experiment in which selected membrane detergents/CHAPS analogues were screened in a short-term real-time stability study. A key aspect of this study is that the detergent concentrations chosen were normalized based on their respective critical micelle concentrations (CMC's), a value specific to each detergent. With respect to maintaining Lp-PLA2 stability, the general trend for the set of detergents was stabilized was maximal when the detergent concentration was at CMC (or higher) with a sharp drop off in stability at concentrations lower than CMC. This result strongly suggests that micelle formation may be important for maintaining Lp-PLA2 stability across the entire panel of detergents surveyed, at least in some situations. When CHAPS was studied to the exclusion of the other detergents, the lots of CHAPS analyzed here actually showed slightly better stability at sub-CMC concentrations than did the other detergents. The 0.595× concentration of CHAPS (corresponding to a standard [4.76 mM] CHAPS concentration in a reference calibrator matrix) showed comparable stability to the 1×CMC concentration, but the stability profile showed ˜10% drop-off at the 0.354× concentration (corresponding to the 2.83 mM CHAPS concentration).

The detergent comparison study also demonstrated that there is differential calibrator stability observed when using different lots of CHAPS detergent. In a comparison of four different lots of CHAPS from two vendors, statistically significant differences in stability were obtained using a Student's t-test even within the timeframe a 30-day short-term stability study. Notably, the difference in stability between the Dojindo lot of CHAPS (lot number CT717) and the Sigma lot #3 (BioXtra, lot number 18K530041V) yielded a statistically significant difference at every CHAPS concentration tested. Given that standard concentrations of other raw materials were used in this study, these results suggest the possibility that differences in stability as a function of detergent concentration can be observed even in a relatively short timeframe. It should be noted, though, that the differential in percent stability observed with some of these lots of Sigma CHAPS (namely, lots 018K53003 and 040M5319V) is of a greater magnitude than that observed in subsequent stability studies with the same two lots of Sigma CHAPS in the Mix-and-Match Study. On the other hand, the single lot of Dojindo CHAPS tested demonstrated good stability at the standard CHAPS concentration and higher when tested using the same pre-formulated master-mixes of the remaining raw materials common to each formulation. The subsequent studies reported here used the BSA grade from the vendor (Millipore Probumin Universal grade) as well as certain raw materials (GMP grade hydrochloric acid) of a potentially higher quality. The role of individual lots of CHAPS detergent was explored in more detail in the Mix- and Match Experiment with a more complete collection of standard raw materials.

A variety of other detergents were screened in the Detergent Comparison study to assess the feasibility of using alternate detergents to stabilize Lp-PLA2. The two CHAPS analogues, CHAPSO and sodium cholate, showed promising short-term stability results. In contrast, two other two CHAPS analogues, BIGCHAP and deoxy-BIGCHAP, were less promising alternatives. The n-octyl-β-glucoside showed some promise with its performance in this initial screen, and it was the detergent used in the determination of the structure of Lp-PLA2 by x-ray crystallography. The n-octyl-β-maltoside showed less promising short term stability indicated by a sharp drop-off in percent stability between the Day 14 time point and the Day 0 time point. The MEGA-8 may be more difficult to use in practice as it requires a relatively high detergent concentration (˜30 mM) for effective protein stabilization. An additional complication is that the current purification scheme utilizes CHAPS throughout the process for isolating recombinant Lp-PLA2 antigen. Switching detergents in the calibrator formulation may require additional validation studies, supplier qualification by QA and/or additional Clinical/Regulatory studies. With these considerations in mind, the remainder of the studies reported here focused exclusively on the CHAPS detergent.

Regarding example 2, the Material Variability Stability Study, this study focused on a strategy of individually substituting the entire battery of raw materials from various grades/vendors into two separate base formulations of raw materials. The base formulations comprised two collections of raw material groupings: a standard grade from current vendors and a test grade of alternative raw material vendors. In addition to the substitution of different raw materials of different grades or from different vendors, multiple lots of the CHAPS (from Sigma/Dojindo vendors) and BSA (from Universal/Diagnostic grade) were also surveyed. These two raw materials are likely candidates to have significant lot-to-lot variability based on both the well-characterized heterogeneities in lots of BSA and the reported trace contamination of CHAPS preparations with precursor molecules. The contaminating precursors of detergents have been shown to completely solubilize into the interior of the detergent micelles, affecting both micelle size and aggregation number. In many cases, the trace contamination of the surfactant preparations with precursor molecules that “show stronger surface activity than that of the main component”, and these “highly surface-active contaminations can affect significantly properties of the system investigated”. The BSA preparations are also subject to a number of well-characterized heterogeneities including variable proportions of dimers and higher oligomers (polymerization/aggregation), mixed disulfide bond formation, IgG contamination, fatty acid contamination, lot-specific protein contaminants. Physico-chemical studies suggest that BSA can adopt different pH-dependent three-dimensional shapes in solution, assuming a prolate ellipsoid (cigar-shaped) conformation at slightly alkaline pH (i.e., 8.3) versus assuming a heart-shaped conformation at neutral pH. Knowledge of which analytical specifications and/or which process variables of the BSA actually affect product performance may be important.

The Material Variation stability study used essentially all standard raw materials for the one grouping of raw materials that is referred to as the “Red Team”. Care was taken to use a particular Universal grade BSA (P/N 81003, L/N 692) as part of the standard grouping of raw materials. The exception is that pharmaceutical grade water (Ricca P/N 9109; USP/Ph. Eur. grade) was used in the standard grade collection of raw materials to mimic the manufacturing protocol used previously. The test grade of raw materials used the HPLC grade water (JTBaker P/N 4218). The Blue Team of raw materials consisted of reagents sourced from different vendors and/or different grades. The effects of these material variations on performance were studied by measuring the responses on both stability and precision using variability gauge analysis.

Main effectors on stability were the lot number of CHAPS and BSA, and these two effectors may interact synergistically. In general, the Red Team, which comprises the standard collection of raw materials, showed better stability than the Blue Team of test raw materials within the designated 100%+/−3% specification used in this analysis. A comparison of the individual formulation conditions within the Red team of raw materials alone indicated CHAPS lot #2 (L/N 040M5319V) and lot #3 (L/N 077K530012) showed the best stability profile across all time points in this study. Within a comparison of the individual formulation conditions within the Blue team of raw materials, these CHAPS lot #4 (Condition #13 using CHAPS L/N 100M53082V) yielded the best stability results, with the caveat that all the Blue Team formulation conditions showed “over-recovery” relative to 100%. Averaged across both the Blue and Red Teams of raw materials, the CHAPS condition #4 showed the best results in the gauge analysis across these two conditions. The results with the CHAPS detergent suggest that it is a critical raw material in the calibrator diluent formulation and some analytical specification or incoming quality control metric may need to be established that is predictive of calibrator stability. The BSA lots also showed dramatic differential effects on stability, with the Millipore Universal BSA “A” (lot 692) and the Millipore Diagnostic Grade “E” (lot 454) demonstrating the best results. It should be noted, however, that the Diagnostic Grade BSA “E” showed the greatest difference in stability in the interaction analysis when Red and Blue teams of raw materials are compared (see FIG. 16C). The other ten combinations of CHAPS/BSA seem to track pretty closely to each other when comparing performance across the Blue and Red teams. Both the CHAPS and BSA demonstrate lot-specific differences, and they do not necessarily correlate with either raw material vendor/grade or any other obvious vendor-provided product specification.

Given the almost identical trending of most of the other raw materials, other raw materials were considered as potential effectors for the Blue team showing more “over-recovery” relative to day Zero compared to the Red team across the study. Two other raw materials, the water grade and the glycerol grade, were considered as candidate effectors of stability as well. Another raw material that is a good candidate for having a deleterious effect on stability is the glycerol. The standard grade of glycerol showed the best performance and was used in the Red team of reagents. Assuming there is little lot-to-lot variability in a standard vendor glycerol, the glycerol is not likely to be a root cause of calibrator instability. However, if there is lot-to-lot variability, then this issue may deserve more consideration moving forward because most glycerol produced today is a by-product of biodiesel and soap production. An additional consideration is the water grade used in the formulation. The indicated water grade was used to formulate most of the raw materials as concentrated solutions before assembling the reactions. As such, the water is confounding variable as the “complementing” water shown in Conditions #30 and #31 comprises only about 30% v/v of the final reaction volume. The better performing Red team of raw materials was formulated with the USP grade water.

The role of raw material variability was also trended for precision. The individual coefficient of variations (% CV) for the twelve timepoints were compared for each of the thirty-six conditions (n=2 replicates/condition/time point). There was a difference of almost 1.4% in the mean coefficient of variation between the best (mean, 1.15%; Condition 22) and worst (mean, 2.54%; Condition 32) of the thirty-six conditions. The standard deviation of the mean % CV also similarly tracked by condition, with the spread between the best (STDEV, 0.55%; Condition 22) and worst (STDEV, 2.48%%; Condition 32) expanding to more than a 1.9% difference. The gauge analysis did not show any dramatic trends in the mean/STDEV of the twelve % CV measurements, although there were some modest individual trends on the mean % CV of the twelve measurements by CHAPS and BSA lot number. Interestingly, when the CHAPS and BSA lots were compared using an interaction analysis performed on the STDEV's of the twelve measurements, these differences expanded. For example, the combination of CHAPS lot 7*BSA lot B (Condition 22) was slightly better than CHAPS lot 1*BSA lot B (Condition 16). In contrast, CHAPS lot 1*BSA lot E (Condition 19) was considerably better than CHAPS lot 7*BSA lot E (Condition 25). One interesting aspect is that the BSA lot E (Diagnostic grade, lot 454) showed significant interaction effects in a CHAPS-lot specific manner for both stability and precision metric in this study. By both metrics, this BSA lot E was considerably better with CHAPS lot #1 than when tested in combination with CHAPS lot #7. These results suggest has the combination of CHAPS lot and BSA lot used in combination may a synergistic effect on calibrator function.

Regarding example 3, the Response Surface Design Stability Study, a type of DOE, known as a response surface design, is useful for generating a map of a response to continuous factors and pinpointing a minimum or maximum response within some specified design space. A popular type of response surface design is the central composite design which combines a factorial design with center points and axial points. The axial points are located a specific distance outside the factor range explored in the factorial design. For a three factor design, it may helpful to conceive of the axial points residing on a sphere that fully engulfs a cube that share a common center point in three dimensions (see FIG. 22A-22B). Thus, the central composite design allows the experimenter to explore five levels of each factor, namely low axial, low factorial, center point, high factorial, and high axial. Here, a rotatable central composite design was utilized to explore five raw material concentrations for four different raw materials (technically, it was just three actual raw materials, but the Tris buffer was independently varied in two dimensions for both Tris buffer concentration and proton concentration [pH]). The full factorial portion of the experiment allowed the design space around the standard concentrations to be explored. The axial point portion of the experiment allowed extreme high and low concentrations to be surveyed, including the potential to “test-to-failure” using low (non-zero) concentrations for two raw materials, CHAPS and DTT. JMP modeling using the “response surface design” fit modeling functionality was utilized to analyze the data.

The CHAPS concentrations were surveyed in a linear concentration range with a standard concentration of 4.76 mM used as the center point concentration. The low factorial concentration selected was 2.83 mM, a reprise of a CHAPS concentration that demonstrated a deleterious effect on stability in a thirty-day Detergent Comparison study. The remaining CHAPS concentrations were chosen mathematically based on these two pre-designated concentrations. The DTT concentrations were surveyed in a linear concentration range with the standard concentration of 0.90 mM used as the high factorial concentration. The remaining four DTT concentrations were selected to explore as broad a range of concentrations between axial concentrations (25-fold) as possible and engineering a potential “test-to-failure” concentration of 0.05 mM. The center point concentration for the DTT was 0.65 mM. Similarly, the Tris buffer concentrations surveyed used the standard concentration as the low factorial point, and the pH range surveyed had the lower/upper specifications of 7.95 and 8.05 incorporated as the midpoint and high factorial concentrations, respectively.

The two raw materials which had the largest effect on stability were the CHAPS and DTT, but they showed different patterns of their respective declines in stability. The axial low concentration of CHAPS demonstrated a sharp and steady decrease in stability observed over the course of the six-month study starting at the first time point on Day 3, with a cumulative 35% drop (e.g., see Condition 12 in FIG. 25). Consistent with the earlier results from the Detergent Comparison study, there was also an immediate 10% drop-off in stability when CHAPS is surveyed at the low factorial concentration of 2.83 mM when assayed at conditions closely approximating the standard formulation (e.g., see Condition 4 in FIG. 25). In contrast, the parallel condition using the high axial CHAPS concentration of 6.69 mM are within the +/−3% specification (see Condition #5 in FIG. 25). These results are consistent with a role for CHAPS micelle formation in maintaining optimal Lp-PLA2 stability. The axial low concentration of DTT showed an initial drop in stability of about 10% over the two initial two timepoints, spanning the first ten days of the study, before stabilizing (e.g., see Condition 11 in FIG. 25). In contrast, the parallel conditions at the midpoint DTT condition (see Conditions 13/14 in FIG. 25) and at the high axial concentration of DTT (see Condition 16 in FIG. 25) are both within the +/−3% specification. This immediate early effect for DTT might be interpreted, contextually, as a labile molecule with a short-half life (temperature- and pH-sensitive) having a role in the reduction some target molecule before rapidly undergoing decomposition in this slightly alkaline buffer conditions. For example, one candidate target molecule might be the Cysteine-34 residue on the solvent interface of the bovine serum albumin, a residue with a free sulfliydryl group prone to oxidation and mixed disulfide formation. Based on JMP modeling of this date set, the JMP prediction profiler predicted optimal concentrations for the following raw materials: The profile predicted optimal stability, (1), trending toward 6.69 mM CHAPS (p=0.004), (2), trending toward a maxima at 0.70 mM DTT (p value <0.0001 for DTT*DTT) and, (3), trending toward pH 8.05 (pH as an effector was only statistically significant when modeled as part of an quadratic interaction with CHAPS or Time; see FIG. 28). Surprisingly, the buffer's concentration, which spanned a concentration range of 5-85 mM, was not a statistically significant effector of stability in this study.

Several anecdotal trends with respect to precision were observed in the Response Surface Design stability study as a function of raw material concentrations, in spite of the fact that the JMP modeling did not show them to be statistically significant effectors. The highest buffer concentration (85 mM) surveyed may improve calibrator precision. This is intriguing because buffer concentration appeared to have no effect on stability, per se, but it may have an effect on precision. Additionally, the axial high concentration tested for CHAPS (8.62 mM) may actually worsen calibrator stability slightly. Considering the axial low proton concentration (pH 8.18) for potential precision improvements may also be an interesting future experimental direction to explore (also, see the pH effects on precision in below).

Regarding Example 4, the Buffer/BSA Survey Stability Study, this Buffer/BSA survey stability study merged two distinct experimental goals. The first goal was to test various permutations of the Tris buffer used in the calibrator formulation to parse out the important aspects of the both the raw material composition and the process for obtaining optimal stability and precision. The permutations included different combinations of different buffer starting raw materials, different buffer concentrations, and process changes involving pre-pH process step, starting pH, and the final pH. A formulation condition was included in this study which mimics previous formulation materials and procedural process. A second goal was to further study the effects on calibrator stability and precision contributed by different grades/lots of Probumin BSA, including Universal Grade, Diagnostic Grade and Fatty Acid-Free Grade. The same lots of both the Universal Grade BSA and Diagnostic Grade BSA that were previously tested in the context of different vendors' CHAPS raw materials in the Material Variation stability study are surveyed here in the context of a fourth distinct lot of Dojindo CHAPS detergent (L/N DC862). For comparison purposes, each of the calibrator formulations were stored both frozen (−70° degrees Celsius) and at refrigerated temperatures (4-8° degrees Celsius) and analyzed in parallel at each time point over the course of four months.

In general, the percent stability was very good across all the buffer composition and process permutations tested. The gauge trending indicates that the buffer composition did not make much of difference with regard to stability. With regard to buffer composition, the Tris base and the Tris-Hydrochloride give essentially equivalent stability. The Tris base:hydrochloride and the pre-formulated Tris base solution both seem to give a slight amount of over-recovery. Very small differences were observed as starting pH and final pH as most of the trend lines fall well within the specification. At both temperatures, the biggest effectors of stability appear to be day-to-day variation and BSA lot number. In addition, this study seems to indicate an increasing percent stability as a function of increasing buffer concentration, at least with the single lot of Teknova-formulated buffer surveyed here. In contrast, no effect was observed on percent stability in the Response Surface Design study as the buffer concentration was increased.

The Buffer/BSA stability study was also analyzed for effects on precision. At both temperatures, the best precision was obtained at pH 8.15 (both starting pH and final pH) relative to the other pH's 8.00 and 7.90. It should be noted that the Tris buffers prepared at pH 8.20 are confounded by their participation in the BSA survey portion of the experimental design as well as the fact that the final pH (8.00) different from the pre-pH (8.20). While the low axial pH sample (8.18) in the Response Surface Experiment showed admirable precision performance in that study, it was not definitively established as the best formulation condition for precision. In addition, it was hampered by the fact that only one formulation was tested in the Response Surface Design study at pH 8.18. The Response Surface Design, on the other hand, had three formulation conditions with a pH set at 8.15. At least in the context of this study, calibrator pH was also shown to have an effect on calibrator precision. In addition, the trending by gauge analysis indicated that BSA lot 454 had the best precision with this lot of CHAPS (L/N DC862) in this study at both temperatures. In the Material Variation stability study, lot 454 (lot E in that study) showed good precision with Sigma CHAPS lot (L/N 018K53003) used in that study, but it showed inferior precision with the Dojindo CHAPS lot (L/N CY785) used. Conversely, the best BSA lot number in that study (referred to as lot 13 in the Material Variation study) showed only average precision performance in this study (referred to as lot 693 in this Buffer/BSA survey study). Taken together, these results suggest that BSA lot number may play a role in calibrator precision, and there might be synergistic effects between the BSA lot and the CHAPS lot used in a given build.

The four real-time stability examples described above summarize the effects of raw material variability and raw material solution concentration surveyed in the calibrator matrix for an ELISA PLAC kit. Based on the results of these studies, the following may improve the robustness of the calibrators with respect to stability and precision and may provide a path for extending an expiration date of a kit. Given that calibrators may be used in any kit (e.g. in an Auto-CAM test), any future improvement of calibrator performance may be tested in a variety of assays, including mass and enzymatic assays, to ensure compatibility, or, alternatively, to fully assess the specific needs of the individual assays with respect to calibrator raw material quality/concentration. The following recommendations include suggestions for incoming quality control analytical testing, in-process test methods and manufacturing validations to maintain, extend and improve calibrator shelf life for the family of Lp-PLA2 analyte-based products.

The CHAPS detergent's quality and its effective solution concentration may be an important quality parameter, and the CHAPS detergent may be an important raw material in a calibrator formulation. Collectively, micelle formation may be important (or essential) for maintaining Lp-PLA2 stability. As discussed above, a trace contamination of CHAPS preparations with their precursor molecules may have deleterious effects on micelle formation of CHAPS. All the lots of CHAPS tested from a given vendor are clearly not functionally equivalent in stability performance in our assay, and it is likely that only certain purities of CHAPS may meet our quality requirements even if they meet the all the vendor's specifications. One possibility is to develop an analytical assay as an incoming quality control test method. This analytical method would be the most facile approach assuming that performance can be shown to depend on control of one or more typical analytical specifications. Such analytical specifications may be one or more of, (1), sufficient to be predictive of CHAPS performance in the calibrator matrix and, (2), be generally applicable, if the detergent should need to be sourced from a secondary vendor. A second possibility is to establish a test method for qualifying performance of CHAPS for use in the calibrator itself similar to the test method used for qualifying BSA. This approach might be more time-consuming, but, nevertheless, it has appeal because it will, (1), provide direct evidence given that the detergent is qualified in the manner in which it will ultimately be used, (2), test the detergent in the context of the other calibrator components, including the BSA, and, (3) allow side-by-side comparison of performance to backlot(s) of CHAPS that will be run as contemporaneous controls. In addition, there may be distinct advantages to using the same lot of CHAPS in antigen purification and the conjugate formulation.

The CHAPS concentration-dependent effects on stability might be a good opportunity for the application of robust design principles (see FIG. 45A). The CHAPS shows a sharp drop-off in stability performance at concentrations less than 2.83 mM. The characterization of the CHAPS concentration-dependent effects indicate a plateau in the stability response above the standard concentration of 4.76 mM with the caveat that the precision got worse at the highest concentration tested of 8.62 mM. The best overall results seem to be achieved at the intermediate concentration of 6.69 mM where the stability and the precision are both excellent. Response of stability is predicted to change very little with small changes in CHAPS concentration around 6.69 mM at the plateau and the variability is minimized due to the excellent % CV. (see FIG. 45B).

The Bovine Serum Albumin may be an important raw material in some formulations. The lot of BSA used can have an effect on both stability and precision of the calibrator. Similar to the CHAPS detergent, the BSA seems to have performance variability that is seemingly more dependent on the lot of raw material chosen than the grade of the material from a given vendor. Some anecdotal and indirect evidence suggests that there may be larger differences in performance variability of the BSA product between vendors. In addition, the evidence presented here suggests that the BSA and the CHAPS may work synergistically together to affect calibrator performance. In addition to the typical BSA product specifications provided (e.g., percent purity, percent protein, pH, sodium and chloride content, IgG contamination, endotoxin level and proteolytic activity), there are a number of well-known heterogeneities in BSA preparations that may affect performance. These might include albumin/SH ratio, N-F transition, secondary and tertiary structures, degree of polymerization, polymer profile, heterologous and homologous polymers, immunochemical protein contaminant profile, pl, fatty acid and lipid profiles and hormone profile. Given the complexity of the BSA purity profile and the unclear relationship in how all these heterogeneities correlate with calibrator performance (if at all), the recommendation here is to maintain the current test method of qualifying the BSA for use in the calibrators, at the very minimum. One lot of BSA was utilized in three of the stability studies presented here, and this lot of BSA showed consistently good performance relative to the some of the other tested lots of BSA. The results presented here suggest that the test method for BSA may be best assayed in the context of the other raw materials to be used in future builds.

DTT is typically used in formulations to maintain the disulfide bonds in protein in a sufficiently reduced state so as to facilitate correct folding while simultaneously avoiding protein aggregate formation. Unfortunately, the DTT is a labile reagent that sensitive to both temperature and pH. In the studies reported here, the DTT concentration seemingly has a threshold effect. The stability response shows a drop-off in stability at the lowest DTT concentration tested in the Response Surface Design study. The results presented here suggest that functional reducing agent is required for calibrator functionality, at least in the early stages. When DTT from two separate vendors was surveyed in the Material Variation study, they functioned comparably. In this study, the functional sulthydryl in calibrator preparations were quantified using the well-known assay using the Ellman's reagent with freshly-prepared free cysteine as the standard. Fresh DTT reagent from both Sigma and BioVectra showed comparable suithydryl activity when assayed on Day 1 of the study (42.5% and 48.4%, respectively, of the initial targeted concentration of 0.95 mM). Although not observed here in this study, there have been anecdotal reports of DTT from certain manufacturers not having the same specific activity from lot-to-lot at the time of assay. The test grade of DTT screened in the Material Variability study was a special cGMP grade available from BioVectra. There may be an opportunity to improve lot-to-lot performance by validating the use of the cGMP grade DTT in the calibrator diluent formulation. In light of some of the previously reported process issues with dissolving the DTT in the presence of high concentrations of BSA, there may be some opportunity to improve and standardize the formulation process by adding freshly-thawed DTT from a concentrated frozen solution. An in-process test method to evaluate the functional sulthydryl activity on the stock DTT reagent and/or on the WIP calibrator diluent could be implemented using the Ellman's reagent prior to the addition of antigen. At first glance, the Ellman's reagent seems to be functional in the context of the calibrator diluent formulation (see Section 8.3.1). The adoption of an in-process test method could involve setting up a capability study to establish a lower specification limit for DTT specific activity at some fixed time after completion of the calibrator diluent formulation work.

Raw materials may be tested prior to use. The test grade glycerol surveyed in this study showed a strong effect in giving “over-recovery” of calibrator stability relative to the standard grade. While this does not suggest that the standard grade of glycerol is the root cause of the calibrator instability problem, there may be an opportunity to utilize a higher quality glycerol reagent to eliminate any possibility of this manifesting itself as an issue due to lot-to-lot variability. Given that almost all glycerol is produced as a byproduct of other manufacturing processes, this type of glycerol can contain animal fats such as beef tallow, and vegetable oils such as coconut, palm kernel, cottonseed, and soybean, which may lead to inferior product stability and significant impurities. One vendor (Dow Optim™ synthetic glycerine) manufactures “synthetic” glycerol that is of USP/cGMP grade and is designed for use in pharmaceutical and biotechnology applications. This pharmaceutical grade synthetic glycerol may be used in the calibrator diluent formulation. Similarly, use of USP/cGMP grade water and cGMP grade hydrochloric acid may be useful. There may also be formulation process changes related to the glycerol that may lead to improvements in calibrator stability, such as adding the glycerol prior to filtration.

An effect of pH on precision was observed in the Buffer/BSA Survey study is also notable. There was a noticeable trend of the precision improving from pH as it became slightly more alkaline, adjusting from pH 7.90 to pH 8.00 to pH 8.15. It is not clear if the starting pH being identical to the final pH is an important parameter: When comparing individual formulations, the two best conditions for precision were Condition 17 (starting pH8.15=final pH8.15) and Condition 18 (starting pH8.20 final pH8.00). Given that the same trends were observed both at both the frozen and refrigerated storage temperature, it is unclear what the exact basis for this precision improvement might be. It is conceivable that precision improvement is assay condition-based and not necessarily the result of improved Lp-PLA2 stability, but these two possibilities are not mutually exclusive. Irrespective of the mechanism, there may be an opportunity to improve the precision of a Lp-PLA2 calibrator function by adjusting the calibrator pH to 8.15.

The results of the four studies summarized in this report have increased the understanding of which raw materials may be important to the achievement of good stability performance and they suggest ranges and optimal values to maximize stability and minimize variation. Such changes may extend calibrator shelf life and result in an overall improvement in product performance for assays, such as a PLAC ELISA and Auto-CAM assay. These improvements may require one or more of the following: refining a calibrator formulation, developing an incoming quality control analytical testing and/or in-process test methods, performing an additional manufacturing validation using high quality (cGMP) raw materials, implementing an additional process control and/or performing capability studies to define specifications. Such a quality-by-design approach may provide tangible product stability improvements so that product shelf life can eventually be extended to nine months, ten months, eleven months, twelve months or more than twelve months expiration dating.

In general, a recombinant Lp-PLA2 may have between about 70 and 100% identity with the amino acid sequence of human Lp-PLA2. For reference, listed below are amino acid sequences of one variation of human Lp-PLA2.

For example, recombinant Lp-PLA2 may be recombinant human Platelet-Activating Factor Acetylhydrolase/PAFAH, and may be produced with a mammalian expression system (e.g., in human cells). The target protein may be expressed with sequence (Phe22-Asn441) of Human PAFAH fused with a polyhistidine tag at the C-terminus (e.g., VDHHHHHH (SEQ ID NO: 4)). In general, Lp-PLA2 may be referred to as Platelet-Activating Factor Acetylhydrolase, PAF Acetylhydrolase, 1-Alkyl-2-Acetylglycerophosphocholine Esterase, 2-Acetyl-1-Alkylglycerophosphocholine Esterase, Group-VIIA Phospholipase A2, gVIIA-PLA2, LDL-Associated Phospholipase A2, LDL-PLA(2), and PAF 2-Ac. SEQ ID NO: 1, below, provides one example of recombinant Lp-PLA2 that may be used as described herein:

rLp-PLA2.1, SEQ ID NO: 1:
FDWQYINPVAHMKSSAWVNKIQVLMAAASFGQTKIPRGNGPYSVGCTDLM
FDHTNKGTFLRLYYPSQDNDRLDTLWIPNKEYFWGLSKFLGTHWLMGNIL
RLLFGSMTTPANWNSPLRPGEKYPLVVFSHGLGAFRTLYSAIGIDLASHG
FIVAAVEHRDRSASATYYFKDQSAAEIGDKSWLYLRTLKQEEETHIRNEQ
VRQRAKECSQALSLILDIDHGKPVKNALDLKEDMEQLKDSIDREKIAVIG
HSFGGATVIQTLSEDQRFRCGIALDAWMFPLGDEVYSRIPQPLFFINSEY
FQYPANIIKMKKCYSPDKERKMITIRGSVHQNFADFTFATGKIIGHMLKL
KGDIDSNVAIDLSNKASLAFLQKHLGLHKDFDQWDCLIEGDDENLIPGTN
INTTNQHIMLQNSSGIEKYN

Another variation of a human recombinant Lp-PLA2 having an N-terminal His-tag fused to the sequence is shown in SEQ ID NO: 2, below.

rLp-PLA2.2, SEQ ID NO: 2:
MGHHHHHHSGSEFELRRQ-
FDWQYINPVAHMKSSAWVNKIQVLMAAASFGQTKIPRGNGPYSVGCTDLM
FDHTNKGTFLRLYYPSQDNDRLDTLWIPNKEYFWGLSKFLGTHWLMGNIL
RLLFGSMTTPANWNSPLRPGEKYPLVVFSHGLGAFRTLYSAIGIDLASHG
FIVAAVEHRDRSASATYYFKDQSAAEIGDKSWLYLRTLKQEEETHIRNEQ
VRQRAKECSQALSLILDIDHGKPVKNALDLKFDMEQLKDSIDREKIAVIG
HSFGGATVIQTLSEDQRFRCGIALDAWMFPLGDEVYSRIPQPLFFINSEY
FQYPANIIKMKKCYSPDKERKMITIRGSVHQNFADFTFATGKIIGHMLKL
KGDIDSNVAIDLSNKASLAFLQKHLGLHKDFDQWDCLIEGDDENLIPGTN
INTTNQHIMLQNSSGIEKYN

Another variation of Recombinant Lp-PLA2 (Phe22˜Asn440) may be expressed in E. coli. This variation is a mouse-derived recombinant protein. Thus, in general, and of the recombinant Lp-PLA2 proteins described herein may be non-human derived Lp-PLA2 proteins (e.g., mouse, rat, dog, horse, etc.). For example, the target protein may be fused with N-terminal His-Tag. The sequence is listed in SEQ ID NO: 3.

rLp-PLA2.3, SEQ ID NO: 2:
MGHHHHHHSGSEFELRRQ-
FHWQDTSSFDFRPSVMFHKLQSVMSAAGSGHSKIPKGNGSYPVGCTDLMF
GYGNESVFVRLYYPAQDQGRLDTVWIPNKEYFLGLSIFLGTPSIVGNILH
LLYGSLTTPASWNSPLRTGEKYPLIVFSHGLGAFRTIYSAIGIGLASNGF
IVATVEHRDRSASATYFFEDQVAAKVENRSWLYLRKVKQEESESVRKEQV
QQRAIECSRALSAILDIEHGDPKENVLGSAFDMKQLKDAIDETKIALMGH
SFGGATVLQALSEDQRFRCGVALDPWMYPVNEELYSRTLQPLLFINSAKF
QTPKDIAKMKKFYQPDKERKMITIKGSVHQNFDDFIFVTGKIIGNKLTLK
GEIDSRVAIDLTNKASMAFLQKHLGLQKDFDQWDPLVEGDDENLIPGSPF
DAVTQVPAQQHSPGSQTQN

Any of the recombinant Lp-PLA2 proteins described herein may include one or more polymorphisms, and in particular known polymorphisms for human Lp-PLA2.

Any of the solutions described herein, and particularly the solutions including recombinant Lp-PLA2 having a long shelf life may be used as a standard, control, calibrator or re-calibrator. For example, any of these solutions may be used to calibrate an assays, such as an assay for detection of Lp-PLA2 activity and/or amount, or it may be used as a control (e.g., a positive control) for an assay for detection of Lp-PLA2 activity or amount.

Positive controls are often used to assess test validity. For example, to assess a test's ability to detect a disease (its sensitivity), then it can be compared against a different test that is already known to work. The well-established test is the positive control, since it has already been established to work. For example, in an enzyme assay to measure the amount of an enzyme (e.g., Lp-PLA2) in a set of extracts, a positive control may be an assay containing a known quantity of the purified enzyme (e.g., recombinant Lp-PLA2) while a negative control would contain no enzyme (e.g., a predetermined concentration of recombinant Lp-PLA2 of zero). The positive control should give a large amount of enzyme activity, while the negative control should give very low to no activity. If the positive control does not produce the expected result, there may be something wrong with the, experimental procedure, and the experiment may be repeated. For difficult or complicated experiments, the result from the positive control can also help in comparison to previous experimental results. For example, if the well-established disease test was determined to have the same effectiveness as found by previous experimenters, this indicates that the experiment is being performed in the same way that the previous experimenters did. Multiple positive controls may be used, which may also allow finer comparisons of the results (calibration, or standardization) if the expected results from the positive controls have different sizes. For example, in the enzyme assay discussed above, a standard curve may be produced by making many different samples with different quantities of the enzyme.

In general, terms such as calibration, calibrators, standards, standardization, reference, control, and re-calibrator are used consistent with the meanings as described in the International Vocabulary of Metrology-Basic and General Concepts and Associated Terms (VIM) (JCGM 200:2012), which is herein incorporated by reference in its entirety.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A value-assigned solution of lipoprotein-associated phospholipase A2 (Lp-PLA2) for use with an Lp-PLA2 assay as a control, standard, calibrator or re-calibrator, the value-assigned solution having a shelf-life of greater than 4 months, the value-assigned solution comprising:
a first predetermined concentration of a recombinant Lp-PLA2 in
a low-salt buffer solution having a salt concentration below about 1 M,
wherein the low-salt buffer solution comprises a detergent forming a plurality of micelles that stabilize the recombinant Lp-PLA2.

2. The value-assigned solution of claim 1, further comprising a second detergent to prevent aggregation of the recombinant Lp-PLA2.

3. The value-assigned solution of claim 2, wherein the second detergent comprises a non-ionic detergent.

4. The value-assigned solution of claim 2, wherein the second detergent comprises a polysorbate detergent.

5. The value-assigned solution of claim 1, wherein the detergent is above a critical micelle concentration (CMC).

6. The value-assigned solution of claim 1, wherein the detergent comprises a cholate detergent.

7. The value-assigned solution of claim 1, wherein the detergent comprises CHAPS.

8. The value-assigned solution of claim 1, wherein the low-salt buffer solution comprises a non-chaotropic salt.

9. The value-assigned solution of claim 1, wherein the low-salt buffer solution comprises one or more of: NaCl and an acetate salt.

10. The value-assigned solution of claim 1, wherein the low-salt buffer solution includes a protein buffered matrix.

11. The value-assigned solution of claim 1, wherein the low-salt buffer solution includes a pH buffer.

12. The value-assigned solution of claim 1, wherein the low-salt buffer solution includes Tris as a pH buffer.

13. A value-assigned solution of lipoprotein-associated phospholipase A2 (Lp-PLA2) for use with an Lp-PLA2 assay as a control, standard, calibrator or re-calibrator, the value-assigned solution having a shelf-life of greater than 4 months, the value-assigned solution comprising:
a first predetermined concentration of a recombinant Lp-PLA2 in
a low-salt buffer solution having a salt concentration below about 1 M,
wherein the low-salt buffer solution comprises a detergent forming a plurality of micelles that stabilize the recombinant Lp-PLA2 and a second detergent to prevent aggregation of the recombinant Lp-PLA2.

14. A lipoprotein-associated phospholipase A2 (Lp-PLA2) kit for use with an Lp-PLA2 assay, the kit having a shelf-life of greater than 4 months, the kit comprising:
a first value-assigned solution comprising a first predetermined concentration of a recombinant Lp-PLA2 in a first low-salt buffer solution having a salt concentration below about 1 M, wherein the first buffer solution comprises a cholate detergent forming a plurality of micelles that stabilizes the recombinant Lp-PLA2, a protein buffered matrix, a pH buffer and a preservative; and
a second value-assigned solution comprising a second low-salt buffer solution having a salt concentration below about 1 M, wherein the second buffer solution comprises a plurality of micelles of the cholate detergent.

15. The kit of claim 14, wherein the cholate detergent comprises CHAPS.

16. The kit of claim 14, wherein the preservative comprises sodium azide.

17. The kit of claim 14, wherein the protein buffered matrix comprises bovine serum albumin (BSA).

18. The kit of claim 14, wherein the first buffer solution comprises a second detergent to prevent aggregation of the recombinant Lp-PLA2.

19. The kit of claim 14, wherein the second buffer solution comprises a second detergent comprising a polysorbate detergent.

20. A lipoprotein-associated phospholipase A2 (Lp-PLA2) assay having recombinant value-assigned solutions having a shelf-life of more than 4 months, the assay comprising:
a plurality of value-assigned solutions each comprising a predetermined concentration of a recombinant Lp-PLA2 in a low-salt buffer solution having a salt concentration below about 1 M, wherein the buffer solution comprises a cholate detergent forming a plurality of micelles that stabilizes the recombinant Lp-PLA2;
a solution comprising an agent that interacts with Lp-PLA2 to produce a detectable signal; and
a wash buffer.

21. The assay of claim 20, further comprising a solid phase support configured to bind Lp-PLA2.

22. The assay of claim 20, wherein the agent comprises a report antibody specific to Lp-PLA2.

23. The assay of claim 20, wherein the low-salt buffer further comprises a second detergent to prevent aggregation of the recombinant Lp-PLA2.

24. The assay of claim 20, wherein the cholate detergent is above a critical micelle concentration (CMC) for the cholate detergent.

25. The assay of claim 20, wherein the cholate detergent comprises CHAPS.

26. The assay of claim 20, wherein the low-salt buffer solution comprises a non-chaotropic salt.

27. The assay of claim 20, wherein the low-salt buffer solution includes a protein buffered matrix.

28. The assay of claim 20, wherein the low-salt buffer solution includes bovine serum albumin (BSA) as a protein buffered matrix.

29. The assay of claim 20, wherein the low-salt buffer solution includes a pH buffer.


SEQUENCE LISTING (4 sequences)

SEQ ID NO: 1 - protein, 420 aa, Artificial Sequence
  Description of Artificial Sequence Synthetic polypeptide
FDWQYINPVAHMKSSAWVNKIQVLMAAASFGQTKIPRGNGPYSVGCTDLMFDHTNKGTFLRLYYPSQDNDRLDTLWIPNKEYFWGLSKFLGTHWLMGNILRLLFGSMTTPANWNSPLRPGEKYPLVVFSHGLGAFRTLYSAIGIDLASHGFIVAAVEHRDRSASATYYFKDQSAAEIGDKSWLYLRTLKQEEETHIRNEQVRQRAKECSQALSLILDIDHGKPVKNALDLKFDMEQLKDSIDREKIAVIGHSFGGATVIQTLSEDQRFRCGIALDAWMFPLGDEVYSRIPQPLFFINSEYFQYPANIIKMKKCYSPDKERKMITIRGSVHQNFADFTFATGKIIGHMLKLKGDIDSNVAIDLSNKASLAFLQKHLGLHKDFDQWDCLIEGDDENLIPGTNINTTNQHIMLQNSSGIEKYN

SEQ ID NO: 2 - protein, 438 aa, Artificial Sequence
  Description of Artificial Sequence Synthetic polypeptide
MGHHHHHHSGSEFELRRQFDWQYINPVAHMKSSAWVNKIQVLMAAASFGQTKIPRGNGPYSVGCTDLMFDHTNKGTFLRLYYPSQDNDRLDTLWIPNKEYFWGLSKFLGTHWLMGNILRLLFGSMTTPANWNSPLRPGEKYPLVVFSHGLGAFRTLYSAIGIDLASHGFIVAAVEHRDRSASATYYFKDQSAAEIGDKSWLYLRTLKQEEETHIRNEQVRQRAKECSQALSLILDIDHGKPVKNALDLKFDMEQLKDSIDREKIAVIGHSFGGATVIQTLSEDQRFRCGIALDAWMFPLGDEVYSRIPQPLFFINSEYFQYPANIIKMKKCYSPDKERKMITIRGSVHQNFADFTFATGKIIGHMLKLKGDIDSNVAIDLSNKASLAFLQKHLGLHKDFDQWDCLIEGDDENLIPGTNINTTNQHIMLQNSSGIEKYN

SEQ ID NO: 3 - protein, 437 aa, Artificial Sequence
  Description of Artificial Sequence Synthetic polypeptide
MGHHHHHHSGSEFELRRQFHWQDTSSFDFRPSVMFHKLQSVMSAAGSGHSKIPKGNGSYPVGCTDLMFGYGNESVFVRLYYPAQDQGRLDTVWIPNKEYFLGLSIFLGTPSIVGNILHLLYGSLTTPASWNSPLRTGEKYPLIVFSHGLGAFRTIYSAIGIGLASNGFIVATVEHRDRSASATYFFEDQVAAKVENRSWLYLRKVKQEESESVRKEQVQQRAIECSRALSAILDIEHGDPKENVLGSAFDMKQLKDAIDETKIALMGHSFGGATVLQALSEDQRFRCGVALDPWMYPVNEELYSRTLQPLLFINSAKFQTPKDIAKMKKFYQPDKERKMITIKGSVHQNFDDFTFVTGKIIGNKLTLKGEIDSRVAIDLTNKASMAFLQKHLGLQKDFDQWDPLVEGDDENLIPGSPFDAVTQVPAQQHSPGSQTQN

SEQ ID NO: 4 - protein, 8 aa, Artificial Sequence
  Description of Artificial Sequence Synthetic peptide
VDHHHHHH